(19) United States
(12) Patent Application Publication
(10) Pub. No.: US 2023/0056288 A1
(43) Pub. Date: Feb. 23, 2023

(54) COMPOSITIONS AND METHODS FOR TREATING AUTOIMMUNE DISEASES AND CANCERS BY TARGETING IGSF8

(71) Applicants: Xiaole Liu, Weston, MA (US); Shanghai XBH Biotechnology Co., LTD., Shanghai (CN); GV20 Therapeutics LLC, Cambridge, MA (US)

(72) Inventors: Xiaole Liu, Weston, MA (US); Xihao Hu, Sudbury, MA (US); Tengfei Xiao, Shanghai (CN)

(21) Appl. No.: 17/789,079

(22) PCT Filed: Dec. 24, 2020
(86) PCT No.: PCT/CN2020/139033

§ 371 Date: Jun. 24, 2022

(30) Foreign Application Priority Data
Dec. 25, 2019 (CN) PCT/CN2019/128294

(51) Int. Cl. C07K 16/28; A61P 35/00

(52) U.S. Cl. CPC C07K 16/2803; A61P 35/00; C07K 2317/24; C07K 2317/55; C07K 2317/622; C07K 2317/565

(57) ABSTRACT

Methods and compositions are provided. The methods and compositions are used for treating a cancer, and/or an autoimmune disease, by modulating the expression and/or activity of IGSF8 and its binding ligands. The pharmaceutical compositions may include, but are not limited to, antibodies that specifically bind human IGSF8, and have an activity of inhibiting IGSF8-mediated immunosuppression in a subject in need thereof.

### REFERENCE TO RELATED APPLICATION

This application claims priority to International Patent Application No. PCT/CN2019/128294, filed on Dec. 25, 2019, the entire content of which, including all drawings and sequence listing, are incorporated herein by reference.

### BACKGROUND OF THE INVENTION

IGSF8 (Immunoglobulin Superfamily Member 8, also known as EWI-2, CD316, and numerous other aliases), encodes a 613-amino acid (or 65 kDa) protein that is a member of the EWI subfamily of the immunoglobulin protein superfamily. This subfamily of proteins all contain a single transmembrane domain, an EWI (Glu-Trp-Ile)-motif (hence the EWI subfamily), and a variable number of immunoglobulin domains.

Human and murine IGSF8 protein sequences are 91% identical. Although IGSF8 transcripts in the two species are expressed in virtually every tissue tested, little is known about the biological function of IGSF8. It has been reported that IGSF8 specifically and directly interacts with the tetraspanins CD81 and CD9 but not with other tetraspanins or with integrins, and it is speculated to regulate the roles of CD9 and CD81 in certain cellular functions, including cell migration and viral infection (Stipp et al., J. Biol. Chem. 276(44):40545-40554, 2001). IGSF8 has also been identified as a potential tumor suppressor, because it has been found to directly interact with another tetraspanin KAI1/CD82, a cancer metastasis suppressor. It has been speculated that IGSF8 is important or likely required for KAI1/CD82-mediated suppression of cancer cell migration (Zhang et al., Cancer Res. 63(10):2665-2674, 2003). IGSF8 has also been found to bind to integrin α4β1 from MOLT-4 T leukemia cells, and it has been suggest that IGSF8-dependent reorganization of α4β1-CD81 complexes on the cell surface is responsible for IGSF8 effects on integrin-dependent morphology and motility functions (Kolesnikova et al., Blood 103(8):3013-3019, 2004). Lastly, IGSF8 has been found to regulate α3β1 integrin-dependent cell function on laminin-5 (Stipp et al., JCB 163(5):1167-1177, 2003).

### SUMMARY OF THE INVENTION

One aspect of the invention provides a method of treating cancer in a subject in need thereof, the method comprising administering to the subject a therapeutically effective amount of an IGSF8 (Immuno Globulin Super Family 8) antagonist.

In a related embodiment, the invention provides a method of stimulating T cell and/or NK cell activation, such as stimulating T cell and/or NK cell activation in tumor microenviroment (TME), the method comprising contacting said T cell and/or NK cell with an IGSF8 (Immuno Globulin Super Family 8) antagonist, such as an antibody or antigen-binding fragment thereof that specifically binds IGSF8.

In another related aspect, the invention provides a use of a therapeutically effective amount of an IGSF8 (Immuno Globulin Super Family 8) antagonist in the manufacture of a medicament for treating cancer in a subject in need thereof.

In another related aspect, the invention provides a composition, such as a pharmaceutical composition, comprising a therapeutically effective amount of an IGSF8 (Immuno Globulin Super Family 8) antagonist, for use in treating cancer in a subject in need thereof.

In certain embodiments, the method, use, composition/pharmaceutical composition for use, further comprises administering to the subject an effective amount of a second therapeutic agent selected from the group consisting of: an immune checkpoint inhibitor, a chemotherapeutic agent, an anti-angiogenesis agent, a growth inhibitory agent, an immune-oncology agent, and an anti-neoplastic composition.

In any one of the above aspects, in certain embodiments, the IGSF8 antagonist is an anti-IGSF8 antibody, or an antigen-binding portion/fragment thereof.

In certain embodiments, the antibody is a chimeric antibody, a humanized antibody, or a human antibody.

In certain embodiments, the antigen-binding portion/fragment is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

In certain embodiments, the cancer is melanoma (including skin cutaneous melanoma), cervical cancer, lung cancer (e.g., non-small cell lung cancer, lung adenocarcinoma, lung squamous cell carcinoma), colorectal cancer, lymphoma (including DLBCL), leukemia (including CLL), BLCA tumor, breast cancer, head-neck squamous cell carcinoma, PRAD, THCA, or UCEC, thyroid cancer, uninary tract cancer, esophagus cancer, liver cancer, or ganglia cancer.

In certain embodiments, the IGSF8 antagonist blocks binding of IGSF8 to a ligand of IGSF8 on a T cell or an NK cell.

In certain embodiments, the IGSF8 antagonist promotes expression, secretion, or otherwise increases activity of a cytokine or a target gene selected from the group consisting of: CXCL10, CXCL9, TNFα, CD81, CD8a, Prfl, IFNγ, Gzma, Gzmb, CD274, PDCD1, PDCD1 Ig2, LAG3, Havcr2, Tigit, or CTLA4.

In certain embodiments, expression, secretion, or otherwise increased activity of the cytokine or the target gene occurs within tumor microenvironment.

In certain embodiments, expression, secretion, or otherwise increased activity of the cytokine or the target gene is due to immune cell (e.g., T lymphocytes or NK cells) infiltration into tumor microenvironment.

In certain embodiments, the IGSF8 antagonist is an immunostimulatory molecule.

In certain embodiments, the IGSF8 antagonist stimulates T cell or NK cell activation and/or infiltration into tumor microenvironment.

In certain embodiments, the immune checkpoint inhibitor is an antibody or antigen-binding fragment thereof specific for PD-1 or PD-L1.

In certain embodiments, the antibody is an anti-PD-1 antibody, such as cemiplimab, nivolumab, or pembrolizumab.

In certain embodiments, the antibody is an anti-PD-L1 antibody, such as avelumab, durvalumab, atezolizumab, KN035, or CK-301.

In certain embodiments, the immune checkpoint inhibitor is a (non-antibody) peptide inhibitor of PD-1/PD-L1, such as AUNP12; a small molecule inhibitor of PD-L1 such as CA-170, or a macrocyclic peptide such as BMS-986189.

Another aspect of the invention provides a use of an IGSF8 antagonist for treating cancer in a subject.

In certain embodiments, the use is for combination use with a second therapeutic agent described herein above.

Another aspect of the invention provides a method of inhibiting binding of IGSF8 to a ligand thereof in a subject, comprising administering to the subject at least one IGSF8 antagonist.

Another aspect of the invention provides a method of inhibiting binding of IGSF8 to a ligand thereof on a cell comprising contacting the cell with at least one IGSF8 antagonist.

In certain embodiments, the cell is contacted in vitro, in vivo, or ex vivo.

Another aspect of the invention provides a composition comprising an IGSF8 antagonist for use in any of the methods of the invention.

Another aspect of the invention provides an antibody which specifically bind IGSF8 for use in a method of treating cancer, preferably through stimulating T cell and/or NK cell activation.

Another aspect of the invention provides an antibody which specifically bind IGSF8 for use in a method of treating cancer, preferably through combination with a second therapeutic agent of the invention.

Another aspect of the invention provides a monoclonal antibody or an antigen-binding fragment thereof specific for IGSF8, wherein said monoclonal antibody comprises: (1) a heavy chain variable region (HCVR), comprising HCVR CDR1-CDR3 sequences of any one of antibodies C1-C29, such as any one of C1-C12; and, (2) a light chain variable region (LCVR), comprising LCVR CDR1-CDR3 sequences of said any one of antibodies C1-C29, such as any one of C1-C12.

In certain embodiments, the monoclonal antibody or antigen-binding fragment thereof comprises: (a) the HCVR sequence of said any one of antibodies C1-C29, such as any one of C1-C12; and/or, (b) the LCVR sequence of said any one of antibodies C1-C29, such as any one of C1-C12.

In certain embodiments, the monoclonal antibody or antigen-binding fragment thereof is a human-mouse chimeric antibody, a humanized antibody, a human antibody, a CDR-grafted antibody, or a resurfaced antibody.

In certain embodiments, the antigen-binding fragment thereof is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

In certain embodiments, the monoclonal antibody or antigen-binding fragment thereof binds IGSF8 with a K_{d} of less than about 25 nM, 20 nM, 15 nM, 10 nM, 5 nM, 2 nM, or 1 nM.

Another aspect of the invention provides a monoclonal antibody or an antigen-binding fragment thereof, which competes with the monoclonal antibody or antigen-binding fragment thereof of the invention for binding to IGSF8.

### BRIEF DESCRIPTION OF THE DRAWINGS

FIG. 1 shows results of a genome-wide natural killer (NK) cell and cancer cell line (colorectal cancer cell line Colo205) co-culture screen, demonstrating that loss of IGSF8 function in Colo205 enhances natural killer (NK) cell cytotoxicity against Colo205. IGSF8 gene is the top 2 hits whose loss sensitized Colo205 cell killing by NK cells.

FIG. 2A shows dose response curves of primary NK cells from human Donor 2 and human Donor 3 treated with human Fc control, or human IGSF8-hFc (human Fc tagged IGSF8). Compared to the Fc control, NK cell viability is significantly reduced as concentration of IGSF8-hFc increases.

FIG. 2B shows dose response curves of primary T cells from human Donor 2 treated with human Fc (hFc) control, or human IGSF8-hFc (human Fc tagged IGSF8). Compared to the hFc control, T cell viability is significantly reduced as concentration of IGSF8-hFc increases.

FIG. 3A shows that CRISPR/Cas9-mediated IGSF8 deletion in the B16-F10 melanoma cells significantly (p<0.0001) reduces the ability of such tumor cells to grow in vivo (as measured by tumor volume in mm³) in a mouse xenograph model (n=8 mice per group). sg IGSF8-1 and -2 represent two experimental groups in which IGSF8 gene was deleted in B16-F10 tumor cells, using two different CRISPR/Cas9 sgRNAs targeting different regions of IGSF8, prior to injection of these IGSF8-deleted B16-F10 tumors into the mice. As a control, the AAV integration site AAVS1 has been deleted similarly in the control B16-F10 tumor cells using sgRNA specific for AAVS1.

FIG. 3B shows that retarded tumor growth in vivo after IGSF8 deletion is not due to difference in relative in vitro cell growth rate of gene-deleted B16-F10 melanoma cells. There is no statistically significant difference in in vitro cell growth rate among the B16-F10 cells deleted of IGSF8, and B16-F10 cells deleted of AAVS1.

FIG. 4 shows that deletion of IGSF8 via CRISPR/Cas9-mediated gene editing in a varieties of cancer cell lines promote CXCL10 expression, which was measured as relative expression fold increase for CXCL10 compared to the same cancer cells deleted of AAVS1. H292 (NCI-H292) is a human mucoepidermoid pulmonary carcinoma cell line; A549 is a human lung carcinoma cell line; Colo205 is a Dukes' type D, colorectal adenocarcinoma cell line; N87 is a human gastric carcinoma cell line; and A375 is a human melanoma cell line.

FIGS. 5A-5D show enhanced relative expression of a varieties of genes in B16-F10 cells (FIGS. 5A and 5C) and tumors (FIGS. 5B and 5D), upon deletion of AAVS1 or IGSF8 by CRISPR/Cas9-mediated gene editing. *: P<0.05; **: P<0.01; ***: P<0.001.

FIG. 6A shows gene expression of IGSF8 in human cancer cell lines (date obtained from the Broad Institute Cancer Cell Line Encyclopedia (CCLE).

FIG. 6B shows statistically significantly elevated expression of IGSF8 in various tumors in The Cancer Genome Atlas (TCGA) cohorts.

FIG. 6C shows clinical relevance of IGSF8 in The Cancer Genome Atlas (TCGA) cohorts. Higher expression of IGSF8 is associated with worse clinical outcome in different cancer types.

FIG. 7 shows binding affinities of representative recombinant anti-IGSF8 antibodies of the invention for the IGSF8 extracellular domain, and EC50 values thereof measured by ELISA.

FIG. 8 shows antibody-dependent cellular cytotoxicity (ADCC) assay and the associated EC50 values for representative anti-IGSF8 antibodies of the invention, using NK cells as effector cells, and A431 cancer cells as target cells.

FIG. 9 shows human CXCL10 ELISA assay for Colo205 cells treated with representative anti-IGSF8 antibodies of the invention (10 μg/mL).

FIG. 10 shows effects of representative anti-IGSF8 monoclonal antibodies of the invention on tumor growth in B16 syngeneic mice. B16-F10 cells were injected subcutaneously into wild type (WT) C57BL/6 mice. Mice were then treated with 2 mg/kg anti-IGSF8 antibodies or control human IgG1 from day 6, every 3 days, for four doses in total. Data are presented as mean±S.E.M. (n=8 mice per group).

FIG. 11 is a line graph showing no significant weight difference among groups of the experimental mice treated with anti-IGSF8 antibodies, or with control human IgG1.

FIG. 12 shows synergistic effect between a subject anti-IGSF8 antibody and an anti-PD-1 antibody in reducing B16-F10 melanoma tumor volume increase in syngeneic mice.

### DETAILED DESCRIPTION OF THE INVENTION

### 1. Overview

The invention described herein is partly based on the discovery that IGSF8 is a novel cancer treatment target, and thus antagonists of IGSF8 can be used to treat such cancer. The data presented herein demonstrate that IGSF8 is uniquely expressed in cancer cells, and is highly expressed in multiple cancer types, particularly in melanoma, cervical cancer, non-small cell lung cancer, and colorectal cancer. IGSF8 interacts with T and NK (natural killer) cells to prevent NK and T cell proliferation and/or reduces the viability of NK and T cells. Meanwhile, knocking out IGSF8 gene or otherwise inactivating IGSF8 function improves tumor infiltration by T and NK cells, and enhances their cytolytic activities in vivo.

Multiple antibodies have been generated against IGSF8, many of which have been validated for IGSF8 binding, blocking, and have exhibited ADCC towards cancer cells expressing IGSF8. More importantly, the data presented herein showed that simultaneously inhibiting IGSF8 function and the PD-1/PD-L1 immune checkpoint led to synergistic efficacy in an in vivo mouse model of cancer (melanoma).

Thus the invention described herein provides methods and reagents for treating cancer by inhibiting IGSF8 activity/antagonizing IGSF8 function, with optional combination with a second therapeutic agent targeting the PD-1/PD-L1 immune checkpoint.

Detailed aspects of the invention are described further and separately in the various sections below. However, it should be understood that any one embodiment of the invention, including embodiments described only in the examples or drawings, and embodiments described only under one section below, can be combined with any other embodiment(s) of the invention.

### 2. Definitions

The term “antibody,” in the broadest sense, encompasses various antibody structures, including but not limited to monoclonal antibodies, polyclonal antibodies, and multispecific antibodies (e.g., bispecific antibodies). The term “antibody” may also broadly refers to a molecule comprising complementarity determining region (CDR) 1, CDR2, and CDR3 of a heavy chain and CDR1, CDR2, and CDR3 of a light chain, wherein the molecule is capable of binding to an antigen. The term “antibody” also includes, but is not limited to, chimeric antibodies, humanized antibodies, human antibodies, and antibodies of various species such as mouse, human, cynomolgus monkey, etc.

In a narrower sense, however, “antibody” refers to the various monoclonal antibodies, including chimeric monoclonal antibodies, humanized monoclonal antibodies, and human monoclonal antibodies.

In some embodiments, an antibody comprises a heavy chain variable region (HCVR) and a light chain variable region (LCVR). In some embodiments, an antibody comprises at least one heavy chain (HC) comprising a heavy chain variable region and at least a portion of a heavy chain constant region, and at least one light chain (LC) comprising a light chain variable region and at least a portion of a light chain constant region. In some embodiments, an antibody comprises two heavy chains, wherein each heavy chain comprises a heavy chain variable region and at least a portion of a heavy chain constant region, and two light chains, wherein each light chain comprises a light chain variable region and at least a portion of a light chain constant region.

As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some such embodiments, the heavy chain is the region of the antibody that comprises the three heavy chain CDRs and the light chain in the region of the antibody that comprises the three light chain CDRs.

The term “heavy chain variable region (HCVR)” as used herein refers to, at a minimum, a region comprising heavy chain CDR1 (CDR-H1), framework 2 (HFR2), CDR2 (CDR-H2), FR3 (HFR3), and CDR3 (CDR-H3). In some embodiments, a heavy chain variable region also comprises at least a portion of an FR1 (HFR1), which is N-terminal to CDR-H1, and/or at least a portion of an FR4 (HFR4), which is C-terminal to CDR-H3.

The term “heavy chain constant region” as used herein refers to a region comprising at least three heavy chain constant domains, CH1, CH2, and CH3. Non-limiting exemplary heavy chain constant regions include γ, δ, and α. Non-limiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, an antibody comprising an α constant region is an IgA antibody, an antibody comprising an c constant region is an IgE antibody, and an antibody comprising an μ constant region is an IgM antibody.

Certain isotypes can be further subdivided into subclasses. For example, IgG antibodies include, but are not limited to, IgG1 (comprising a γ1 constant region), IgG2 (comprising a γ2 constant region), IgG3 (comprising a γ3 constant region), and IgG4 (comprising a γ4 constant region) antibodies; IgA antibodies include, but are not limited to, IgA1 (comprising an al constant region) and IgA2 (comprising an α2 constant region) antibodies; and IgM antibodies include, but are not limited to, IgM1 (comprising an μ1 constant region) and IgM2 (comprising an μ2 constant region).

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence, and with or without a C-terminal lysine.

The term “light chain variable region (LCVR)” as used herein refers to a region comprising light chain CDR1 (CDR-L1), framework (FR) 2 (LFR2), CDR2 (CDR-L2), FR3 (LFR3), and CDR3 (CDR-L3). In some embodiments, a light chain variable region also comprises at least a portion of an FR1 (LFR1) and/or at least a portion of an FR4 (LFR4).

The term “light chain constant region” as used herein refers to a region comprising a light chain constant domain, C_{L}. Non-limiting exemplary light chain constant regions include λ, and κ.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

The term “antibody fragment” or “antigen binding portion” (of antibody) includes, but is not limited to, fragments that are capable of binding antigen, such as Fv, single-chain Fv (scFv), Fab, Fab′, and (Fab′)₂.

An “antibody that binds to the same epitope” as a reference antibody can be determined by an antibody competition assay. It refers to an antibody that blocks binding of the reference antibody to its antigen in a competition assay by 50% or more, and conversely, the reference antibody blocks binding of the antibody to its antigen in a competition assay by 50% or more. The term “compete” when used in the context of an antibody that compete for the same epitope means competition between antibodies is determined by an assay in which an antibody being tested prevents or inhibits specific binding of a reference antibody to a common antigen.

Numerous types of competitive binding assays can be used, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619); solid phase direct labeled assay; solid phase direct labeled sandwich assay (see, e.g., Harlow and Lane, 1988, Antibodies, A Laboratory Manual, Cold Spring Harbor Press); solid phase direct label RIA using I¹²⁵ label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol.).

Typically, such an assay involves the use of purified antigen bound to a solid surface or cells bearing either of these, an unlabeled test antigen binding protein and a labeled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibodies and antibodies binding to an adjacent epitope sufficiently proximal to the epitope bound by the reference antibody for steric hindrance to occur. In some embodiments, when a competing antibody is present in excess, it will inhibit specific binding of a reference antibody to a common antigen by at least 40%, 45%, 50%, 55%, 60%, 65%, 70% or 75%. In some instance, binding is inhibited by at least 80%, 85%, 90%, 95%, or 97% or more.

The term “antigen” refers to a molecule or a portion of a molecule capable of being bound by a selective binding agent, such as an antibody or immunologically functional fragment thereof, and additionally capable of being used in a mammal to produce antibodies capable of binding to that antigen. An antigen may possess one or more epitopes that are capable of interacting with antibodies.

The term “epitope” is the portion of an antigen molecule that is bound by a selective binding agent, such as an antibody or a fragment thereof. The term includes any determinant capable of specifically binding to an antibody. An epitope can be contiguous or non-contiguous (e.g., in a polypeptide, amino acid residues that are not contiguous to one another in the polypeptide sequence but that within in context of the molecule are bound by the antigen binding protein). In some embodiments, epitopes may be mimetic in that they comprise a three dimensional structure that is similar to an epitope used to generate the antibody, yet comprise none or only some of the amino acid residues found in that epitope used to generate the antibody. Epitope determinants may include chemically active surface groupings of molecules such as amino acids, sugar side chains, phosphoryl or sulfonyl groups, and may have specific three dimensional structural characteristics, and/or specific charge characteristics.

In some embodiments, an “epitope” is defined by the method used to determine it. For example, in some embodiments, an antibody binds to the same epitope as a reference antibody, if they bind to the same region of the antigen, as determined by hydrogen-deuterium exchange (HDX).

In certain embodiments, an antibody binds to the same epitope as a reference antibody if they bind to the same region of the antigen, as determined by X-ray crystallography.

A “chimeric antibody” as used herein refers to an antibody comprising at least one variable region from a first species (such as mouse, rat, cynomolgus monkey, etc.) and at least one constant region from a second species (such as human, cynomolgus monkey, chicken, etc.). In some embodiments, a chimeric antibody comprises at least one mouse variable region and at least one human constant region. In some embodiments, all of the variable regions of a chimeric antibody are from a first species and all of the constant regions of the chimeric antibody are from a second species.

A “humanized antibody” as used herein refers to an antibody in which at least one amino acid in a framework region of a non-human variable region (such as mouse, rat, cynomolgus monkey, chicken, etc.) has been replaced with the corresponding amino acid from a human variable region. In some embodiments, a humanized antibody comprises at least one human constant region or fragment thereof. In some embodiments, a humanized antibody fragment is an Fab, an scFv, a (Fab′)₂, etc.

A “CDR-grafted antibody” as used herein refers to a humanized antibody in which one or more complementarity determining regions (CDRs) of a first (non-human) species have been grafted onto the framework regions (FRs) of a second (human) species.

A “human antibody” as used herein refers to antibodies produced in humans, antibodies produced in non-human animals that comprise human immunoglobulin genes, such as XENOMOUSE®, and antibodies selected using in vitro methods, such as phage display, wherein the antibody repertoire is based on a human immunoglobulin sequences.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Non-limiting exemplary mammalian cells include, but are not limited to, NSO cells, PER.C6® cells (Crucell), and 293 and CHO cells, and their derivatives, such as 293-6E and DG44 cells, respectively.

The term “isolated” as used herein refers to a molecule that has been separated from at least some of the components with which it is typically found in nature or has been separated from at least some of the components with which it is typically produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated” so long as that polynucleotide is not found in that vector in nature.

The terms “subject” and “patient” are used interchangeably herein to refer to a mammal such as human. In some embodiments, methods of treating other non-human mammals, including, but not limited to, rodents, simians, felines, canines, equines, bovines, porcines, ovines, caprines, mammalian laboratory animals, mammalian farm animals, mammalian sport animals, and mammalian pets, are also provided. In some instances, a “subject” or “patient” refers to a (human) subject or patient in need of treatment for a disease or disorder.

The term “sample” or “patient sample” as used herein, refers to material that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized.

By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as sputum, cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample,” “reference cell,” or “reference tissue,” as used herein, refers to a sample, cell or tissue obtained from a source known, or believed, not to be afflicted with the disease or condition for which a method or composition of the invention is being used to identify. In one embodiment, a reference sample, reference cell or reference tissue is obtained from a healthy part of the body of the same subject or patient in whom a disease or condition is being identified using a composition or method of the invention. In one embodiment, a reference sample, reference cell or reference tissue is obtained from a healthy part of the body of at least one individual who is not the subject or patient in whom a disease or condition is being identified using a composition or method of the invention. In some embodiments, a reference sample, reference cell or reference tissue was previously obtained from a patient prior to developing a disease or condition or at an earlier stage of the disease or condition.

A “disorder” or “disease” is any condition that would benefit from treatment with one or more IGSF8 antagonists of the invention. This includes chronic and acute disorders or diseases including those pathological conditions that predispose the mammal to the disorder in question. Non-limiting examples of disorders to be treated herein include cancers.

The term “cancer” is used herein to refer to a group of cells that exhibit abnormally high levels of proliferation and growth. A cancer may be benign (also referred to as a benign tumor), pre-malignant, or malignant. Cancer cells may be solid cancer cells (i.e., forming solid tumors) or leukemic cancer cells. The term “cancer growth” is used herein to refer to proliferation or growth by a cell or cells that comprise a cancer that leads to a corresponding increase in the size or extent of the cancer.

Examples of cancer include but are not limited to, carcinoma, lymphoma, blastoma, sarcoma, and leukemia. More particular nonlimiting examples of such cancers include squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer.

A “chemotherapeutic agent” is a chemical compound that can be useful in the treatment of cancer. Examples of chemotherapeutic agents include, but are not limited to, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem Intl. Ed. Engl, 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib))(TARCEVA® and VEGF-A that reduce cell proliferation and pharmaceutically acceptable salts, acids or derivatives of any of the above.

Further non-limiting exemplary chemotherapeutic agents include anti-hormonal agents that act to regulate or inhibit hormone action on cancers such as anti-estrogens and selective estrogen receptor modulators (SERMs), including, for example, tamoxifen (including NOLVADEX® tamoxifen), raloxifene, droloxifene, 4-hydroxy tamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

An “anti-angiogenesis agent” or “angiogenesis inhibitor” refers to a small molecular weight substance, a polynucleotide (including, e.g., an inhibitory RNA (RNAi or siRNA)), a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent, e.g., antibodies to VEGF-A (e.g., bevacizumab (AVASTIN®)) or to the VEGF-A receptor (e.g., KDR receptor or Flt-1 receptor), anti-PDGFR inhibitors such as GLEEVEC® (Imatinib Mesylate), small molecules that block VEGF receptor signaling (e.g., PTK787/ZK2284, SU6668, SUTENT®/SUl 1248 (sunitinib malate), AMG706, or those described in, e.g., international patent application WO 2004/113304). Anti-angiogensis agents also include native angiogenesis inhibitors, e.g., angiostatin, endostatin, etc. See, e.g., Klagsbrun and D'Amore (1991) Annu. Rev. Physiol. 53:217-39; Streit and Detmar (2003) Oncogene 22:3172-3179 (e.g., Table 3 listing anti-angiogenic therapy in malignant melanoma); Ferrara & Alitalo (1999) Nature Medicine 5(12): 1359-1364; Tonini et al. (2003) Oncogene 22:6549-6556 (e.g., Table 2 listing known anti-angiogenic factors); and, Sato (2003) Int. J. Clin. Oncol. 8:200-206 (e.g., Table 1 listing anti-angiogenic agents used in clinical trials).

A “growth inhibitory agent” as used herein refers to a compound or composition that inhibits growth of a cell (such as a cell expressing VEGF) either in vitro or in vivo. Thus, the growth inhibitory agent may be one that significantly reduces the percentage of cells (such as a cell expressing VEGF) in S phase. Examples of growth inhibitory agents include, but are not limited to, agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

The term “anti-neoplastic composition” refers to a composition useful in treating cancer comprising at least one active therapeutic agent. Examples of therapeutic agents include, but are not limited to, e.g., chemotherapeutic agents, growth inhibitory agents, cytotoxic agents, agents used in radiation therapy, anti-angiogenesis agents, cancer immunotherapeutic agents (also referred to as immuno-oncology agents), apoptotic agents, anti-tubulin agents, and other-agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (TARCEVA®), platelet derived growth factor inhibitors (e.g., GLEEVEC® (Imatinib Mesylate)), a COX-2 inhibitor (e.g., celecoxib), interferons, CTLA4 inhibitors (e.g., anti-CTLA antibody ipilimumab (YERVOY®)), PD-1 inhibitors (e.g., anti-PD1 antibodies, BMS-936558), PDL1 inhibitors (e.g., anti-PDL1 antibodies, MPDL3280A), PDL2 inhibitors (e.g., anti-PDL2 antibodies), VISTA inhibitors (e.g., anti-VISTA antibodies), cytokines, antagonists (e.g., neutralizing antibodies) that bind to one or more of the following targets ErbB2, ErbB3, ErbB4, PDGFR-beta, BlyS, APRIL, BCMA, PD-1, PDL1, PDL2, CTLA4, VISTA, or VEGF receptor(s), TRAIL/Apo2, and other bioactive and organic chemical agents, etc. Combinations thereof are also included in the invention.

“Treatment” refers to therapeutic treatment, for example, wherein the object is to slow down (lessen) the targeted pathologic condition or disorder as well as, for example, wherein the object is to inhibit recurrence of the condition or disorder. “Treatment” covers any administration or application of a therapeutic for a disease (also referred to herein as a “disorder” or a “condition”) in a mammal, including a human, and includes inhibiting the disease or progression of the disease, inhibiting or slowing the disease or its progression, arresting its development, partially or fully relieving the disease, partially or fully relieving one or more symptoms of a disease, or restoring or repairing a lost, missing, or defective function; or stimulating an inefficient process. The term “treatment” also includes reducing the severity of any phenotypic characteristic and/or reducing the incidence, degree, or likelihood of that characteristic. Those in need of treatment include those already with the disorder as well as those at risk of recurrence of the disorder or those in whom a recurrence of the disorder is to be prevented or slowed down.

The term “effective amount” or “therapeutically effective amount” refers to an amount of a drug effective to treat a disease or disorder in a subject. In some embodiments, an effective amount refers to an amount effective, at dosages and for periods of time necessary, to achieve the desired therapeutic or prophylactic result. A therapeutically effective amount of IGSF8 antagonist of the invention may vary according to factors such as the disease state, age, sex, and weight of the individual, and the ability of the antagonist to elicit a desired response in the individual. A therapeutically effective amount encompasses an amount in which any toxic or detrimental effects of IGSF8 antagonist are outweighed by the therapeutically beneficial effects.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

An “article of manufacture” is any manufacture (e.g., a package or container) or kit comprising at least one reagent, e.g., a medicament for treatment of a disease or disorder, or a probe for specifically detecting a biomarker described herein. In some embodiments, the manufacture or kit is promoted, distributed, or sold as a unit for performing the methods described herein.

### 3. Methods of Treating Cancer

The invention described herein provides IGSF8 antagonists for use in methods of treating humans and other non-human mammals.

In some embodiments, methods for treating or preventing a cancer are provided, comprising administering an effective amount of IGSF8 antagonist to a subject in need of such treatment.

In some embodiments, methods of treating cancer are provided, wherein the methods comprise administering IGSF8 antagonist to a subject with cancer.

In some embodiments, use of IGSF8 antagonist for treating cancer is provided.

Non-limiting exemplary cancers that may be treated with IGSF8 antagonists are provided herein, including carcinoma, lymphoma, blastoma, sarcoma, and leukemia. More particular non-limiting examples of such cancers include melanoma, cervical cancer, squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, ovarian cancer, liver cancer, bladder cancer, hepatoma, breast cancer, colon cancer, colorectal cancer, endometrial or uterine carcinoma, salivary gland carcinoma, kidney cancer, renal cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer.

In some embodiments, lung cancer is non-small cell lung cancer or lung squamous cell carcinoma.

In some embodiments, leukemia is acute myeloid leukemia (AML) or chronic lymphocytic leukemia (CLL).

In some embodiments, breast cancer is breast invasive carcinoma.

In some embodiments, ovarian cancer is ovarian serous cystadenocarcinoma.

In some embodiments, kidney cancer is kidney renal clear cell carcinoma.

In some embodiments, colon cancer is colon adenocarcinoma.

In some embodiments, bladder cancer is bladder urothelial carcinoma.

In some embodiments, the IGSF8 antagonist is selected from a IGSF8 antibody.

In some embodiments, the IGSF8 antagonist for treating cancer may be a non-antibody protein, such as a soluble version of the IGSF8 protein or a portion thereof (e.g., the ECD) that inhibits the interaction between IGSF8 and its ligand, optionally further comprising a fusion partner and in the form of a fusion molecule. Various exemplary IGSF8 antagonists are described in more detail in the sections that follow.

### 4. Routes of Administration and Carriers

In various embodiments, IGSF8 antagonists may be administered subcutaneously or intravenously.

In some embodiments, IGSF8 antagonist may be administered in vivo by various routes, including, but not limited to, oral, intra-arterial, parenteral, intranasal, intramuscular, intracardiac, intraventricular, intratracheal, buccal, rectal, intraperitoneal, by inhalation, intradermal, topical, transdermal, and intrathecal, or otherwise, e.g., by implantation.

The subject compositions may be formulated into preparations in solid, semi-solid, liquid, or gaseous forms; including, but not limited to, tablets, capsules, powders, granules, ointments, solutions, suppositories, enemas, injections, inhalants, and aerosols.

In some embodiments, IGSF8 antagonist is delivered using gene therapy. As a non-limiting example, a nucleic acid molecule encoding IGSF8 antagonist (such as Cas9 and sgRNA, or Cas12a and crRNA) may be coated onto gold microparticles and delivered intradermally by a particle bombardment device, or “gene gun,” e.g., as described in the literature (see, e.g., Tang et al, Nature 356: 152-154 (1992)).

In various embodiments, compositions comprising IGSF8 antagonist are provided in formulations with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Nonlimiting exemplary carriers include saline, buffered saline, dextrose, water, glycerol, ethanol, and combinations thereof.

In various embodiments, compositions comprising IGSF8 antagonist may be formulated for injection, including subcutaneous administration, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives.

In various embodiments, the compositions may be formulated for inhalation, for example, using pressurized acceptable propellants such as dichlorodifiuoromethane, propane, nitrogen, and the like.

The compositions may also be formulated, in various embodiments, into sustained release microcapsules, such as with biodegradable or non-biodegradable polymers. A non-limiting exemplary biodegradable formulation includes poly lactic acid-glycolic acid (PLGA) polymer. A non-limiting exemplary non-biodegradable formulation includes a polyglycerin fatty acid ester. Certain methods of making such formulations are described, for example, in EP 1125584 A1.

Pharmaceutical dosage packs comprising one or more containers, each containing one or more doses of IGSF8 antagonist, are also provided. In some embodiments, a unit dosage is provided wherein the unit dosage contains a predetermined amount of a composition comprising IGSF8 antagonist, with or without one or more additional agents. In some embodiments, such a unit dosage is supplied in single-use prefilled syringe for injection. In various embodiments, the composition contained in the unit dosage may comprise saline, sucrose, or the like; a buffer, such as phosphate, or the like; and/or be formulated within a stable and effective pH range. Alternatively, in some embodiments, the composition may be provided as a lyophilized powder that may be reconstituted upon addition of an appropriate liquid, for example, sterile water. In some embodiments, the composition comprises one or more substances that inhibit protein aggregation, including, but not limited to, sucrose and arginine. In some embodiments, a composition of the invention comprises heparin and/or a proteoglycan.

Pharmaceutical compositions are administered in an amount effective for treatment or prophylaxis of the specific indication. The therapeutically effective amount is typically dependent on the weight of the subject being treated, his or her physical or health condition, the extensiveness of the condition to be treated, or the age of the subject being treated.

In some embodiments, IGSF8 antagonist may be administered in an amount in the range of about 50 μg/kg body weight to about 50 mg/kg body weight per dose. In some embodiments, IGSF8 antagonist may be administered in an amount in the range of about 100 μg/kg body weight to about 50 mg/kg body weight per dose. In some embodiments, IGSF8 antagonist may be administered in an amount in the range of about 100 μg/kg body weight to about 20 mg/kg body weight per dose. In some embodiments, IGSF8 antagonist may be administered in an amount in the range of about 0.5 mg/kg body weight to about 20 mg/kg body weight per dose.

In some embodiments, IGSF8 antagonist may be administered in an amount in the range of about 10 mg to about 1,000 mg per dose. In some embodiments, IGSF8 antagonist may be administered in an amount in the range of about 20 mg to about 500 mg per dose. In some embodiments, IGSF8 antagonist may be administered in an amount in the range of about 20 mg to about 300 mg per dose. In some embodiments, IGSF8 antagonist may be administered in an amount in the range of about 20 mg to about 200 mg per dose.

The IGSF8 antagonist compositions may be administered as needed to subjects. In some embodiments, an effective dose of IGSF8 antagonist is administered to a subject one or more times. In various embodiments, an effective dose of IGSF8 antagonist is administered to the subject once a month, less than once a month, such as, for example, every two months, every three months, or every six months. In other embodiments, an effective dose of IGSF8 antagonist is administered more than once a month, such as, for example, every two weeks, every week, twice per week, three times per week, daily, or multiple times per day. An effective dose of IGSF8 antagonist is administered to the subject at least once. In some embodiments, the effective dose of IGSF8 antagonist may be administered multiple times, including for periods of at least a month, at least six months, or at least a year. In some embodiments, IGSF8 antagonist is administered to a subject as-needed to alleviate one or more symptoms of a condition.

### 5. Combination Therapy

IGSF8 antagonists of the invention, including any antibodies and functional fragments thereof, may be administered to a subject in need thereof in combination with other biologically active substances or other treatment procedures for the treatment of diseases. For example, IGSF8 antagonists may be administered alone or with other modes of treatment. They may be provided before, substantially contemporaneous with, or after other modes of treatment, such as radiation therapy.

For treatment of cancer, the IGSF8 antagonist may be administered in conjunction with one or more of anti-cancer agents, such as the immune checkpoint inhibitor, chemotherapeutic agent, growth inhibitory agent, anti-angiogenesis agent or anti-neoplastic composition.

In certain embodiments, IGSF8 antagonist specifically binds to IGSF8 (an “IGSF8-binding antagonist”), e.g., IGSF8 antagonist antibody or antigen-binding fragment thereof, is administered with a second antagonist such as an immune checkpoint inhibitor (e.g., an inhibitor of the PD-1 or PD-L1 pathway), to a subject having a disease in which the stimulation of the immune system would be beneficial, e.g., cancer or infectious diseases. The two antagonists may be administered simultaneously or consecutively, e.g., as described below for the combination of IGSF8 antagonist with an immuno-oncology agent. One or more additional therapeutics, e.g., checkpoint modulators may be added to a treatment with IGSF8 binding antagonist for treating cancer or infectious diseases.

In certain embodiments, IGSF8 antagonist is administered with another treatment, either simultaneously, or consecutively, to a subject, e.g., a subject having cancer. For example, IGSF8 antagonist may be administered with one of more of: radiotherapy, surgery, or chemotherapy, e.g., targeted chemotherapy or immunotherapy.

Immunotherapy, e.g., cancer immunotherapy includes cancer vaccines and immuno-oncology agents. IGSF8 antagonist may be, e.g., a protein, an antibody, antibody fragment or a small molecule, that binds to IGSF8. IGSF8 antagonist may be an antibody or antigen binding fragment thereof that specifically binds to IGSF8.

In certain embodiments, a method of treatment of a subject having cancer comprises administering to the subject having the cancer IGSF8 antagonist, e.g., IGSF8 antibody, and one or more immuno-oncology agents, such as immune checkpoint inhibitor.

Immunotherapy, e.g., therapy with an immuno-oncology agent, is effective to enhance, stimulate, and/or upregulate immune responses in a subject. In one aspect, the administration of IGSF8 antagonist with an immuno-oncology agent (such as a PD-1 inhibitor) has a synergic effect in the treatment of cancer, e.g., in inhibiting tumor growth.

In one aspect, IGSF8 antagonist is sequentially administered prior to administration of the immuno-oncology agent. In one aspect, IGSF8 antagonist is administered concurrently with the immunology-oncology agent (such as PD-1 inhibitor). In yet one aspect, IGSF8 antagonist is sequentially administered after administration of the immuno-oncology agent (such as PD-1 inhibitor). The administration of the two agents may start at times that are, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks apart, or administration of the second agent may start, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks after the first agent has been administered.

In certain aspects, IGSF8 antagonist and an immuno-oncology agent (e.g., PD-1 inhibitor) are administered simultaneously, e.g., are infused simultaneously, e.g., over a period of 30 or 60 minutes, to a patient. IGSF8 antagonist may be co-formulated with an immuno-oncology agent (such as PD-1 inhibitor).

Immuno-oncology agents include, for example, a small molecule drug, antibody or fragment thereof, or other biologic or small molecule. Examples of biologic immuno-oncology agents include, but are not limited to, antibodies, antibody fragments, vaccines and cytokines. In one aspect, the antibody is a monoclonal antibody. In certain aspects, the monoclonal antibody is humanized or human antibody.

In one aspect, the immuno-oncology agent is (i) an agonist of a stimulatory (including a co-stimulatory) molecule (e.g., receptor or ligand) or (ii) an antagonist of an inhibitory (including a co-inhibitory) molecule (e.g., receptor or ligand) on immune cells, e.g., T cells, both of which result in amplifying antigen-specific T cell responses. In certain aspects, an immuno-oncology agent is (i) an agonist of a stimulatory (including a co-stimulatory) molecule (e.g., receptor or ligand) or (ii) an antagonist of an inhibitory (including a co-inhibitory) molecule (e.g., receptor or ligand) on cells involved in innate immunity, e.g., NK cells, and wherein the immuno-oncology agent enhances innate immunity. Such immuno-oncology agents are often referred to as immune checkpoint regulators, e.g., immune checkpoint inhibitor or immune checkpoint stimulator.

In certain embodiments, an immuno-oncology agent targets a stimulatory or inhibitory molecule that is a member of the immunoglobulin super family (IgSF). For example, an immuno-oncology agent may be an agent that targets (or binds specifically to) a member of the B7 family of membrane-bound ligands, which includes B7-1, B7-2, B7-H1 (PD-L1), B7-DC (PD-L2), B7-H2 (ICOS-L), B7-H3, B7-H4, B7-H5, and B7-H6, or a co-stimulatory or co-inhibitory receptor binding specifically to a B7 family member. An immuno-oncology agent may be an agent that targets a member of the TNF family of membrane bound ligands or a co-stimulatory or co-inhibitory receptor binding specifically thereto, e.g., a TNF receptor family member. Exemplary TNF and TNFR family members that may be targeted by immuno-oncology agents include CD40 and CD40L, OX-40, OX-40L, GITR, GITRL, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTfiR, LIGHT, DcR3, HVEM, VEGI/TL1A, TRAMP/DR3, EDAR, EDA1, XEDAR, EDA2, TNFR1, Lymphotoxin α/TNPβ, TNFR2, TNFα, LTfiR, Lymphotoxin a 1β2, FAS, FASL, RELT, DR6, TROY and NGFR. An immuno-oncology agent that may be used in combination with IGSF8 antagonist agent for treating cancer may be an agent, e.g., an antibody, targeting an IgSF member, such as a B7 family member, a B7 receptor family member, a TNF family member or a TNFR family member, such as those described above.

In one aspect, IGSF8 antagonist is administered with one or more of (i) an antagonist of a protein that inhibits T cell activation (e.g., immune checkpoint inhibitor) such as CTLA-4, PD-1, PD-L1, PD-L2, LAG-3, TIM3, Galectin 9, CEACAM-1, BTLA, CD69, Galectin-1, TIGIT, CD113, GPR56, VISTA, B7-H3, B7-H4, 2B4, CD48, GARP, PDIH, LAIR1, TIM-1, TIM-4, and PSGL-1 and (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, CD40L, DR3 and CD28H.

In one aspect, an immuno-oncology agent is an agent that inhibits (i.e., an antagonist of) a cytokine that inhibits T cell activation (e.g., IL-6, IL-10, TGF-β, VEGF, and other immunosuppressive cytokines) or is an agonist of a cytokine, such as IL-2, IL-7, IL-12, IL-15, IL-21 and IFNα (e.g., the cytokine itself) that stimulates T cell activation, and stimulates an immune response.

Other agents that can be combined with IGSF8 antagonist for stimulating the immune system, e.g., for the treatment of cancer and infectious diseases, include antagonists of inhibitory receptors on NK cells or agonists of activating receptors on NK cells. For example, Anti-IGSF8 antagonist can be combined with an antagonist of KIR.

Yet other agents for combination therapies include agents that inhibit or deplete macrophages or monocytes, including but not limited to CSF-IR antagonists such as CSF-IR antagonist antibodies including RG7155 (WO1 1/70024, WO1 1/107553, WO11/131407, W013/87699, W013/119716, WO13/132044) or FPA008 (WO1 1/140249; W013169264; WO14/036357).

Immuno-oncology agents also include agents that inhibit TGF-β signaling.

Additional agents that may be combined with IGSF8 antagonist include agents that enhance tumor antigen presentation, e.g., dendritic cell vaccines, GM-CSF secreting cellular vaccines, CpG oligonucleotides, and imiquimod, or therapies that enhance the immunogenicity of tumor cells (e.g., anthracyclines).

Yet other therapies that may be combined with IGSF8 antagonist include therapies that deplete or block Treg cells, e.g., an agent that specifically binds to CD25.

Another therapy that may be combined with IGSF8 antagonist is a therapy that inhibits a metabolic enzyme such as indoleamine dioxigenase (IDO), dioxigenase, arginase, or nitric oxide synthetase.

Another class of agents that may be used includes agents that inhibit the formation of adenosine or inhibit the adenosine A2A receptor.

Other therapies that may be combined with IGSF8 antagonist for treating cancer include therapies that reverse/prevent T cell anergy or exhaustion and therapies that trigger an innate immune activation and/or inflammation at a tumor site.

IGSF8 antagonist may be combined with more than one immuno-oncology agent (such as immune checkpoint inhibitor), and may be, e.g., combined with a combinatorial approach that targets multiple elements of the immune pathway, such as one or more of the following: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Treg or other immune suppressing cells; a therapy that stimulates positive immune regulation, e.g., with agonists that stimulate the CD-137, OX-40 and/or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxigenase (IDO), dioxigenase, arginase or nitric oxide synthetase; a therapy that reverses/prevents T cell anergy or exhaustion; a therapy that triggers an innate immune activation and/or inflammation at a tumor site; administration of immune stimulatory cytokines or blocking of immuno repressive cytokines.

For example, IGSF8 antagonist can be used with one or more agonistic agents that ligate positive costimulatory receptors; one or more antagonists (blocking agents) that attenuate signaling through inhibitory receptors, such as antagonists that overcome distinct immune suppressive pathways within the tumor microenvironment (e.g., block PD-L1/PD-1/PD-L2 interactions); one or more agents that increase systemically the frequency of anti-tumor immune cells, such as T cells, deplete or inhibit Tregs (e.g., by inhibiting CD25); one or more agents that inhibit metabolic enzymes such as IDO; one or more agents that reverse/prevent T cell anergy or exhaustion; and one or more agents that trigger innate immune activation and/or inflammation at tumor sites.

In one embodiment, a subject having a disease that may benefit from stimulation of the immune system, e.g., cancer or an infectious disease, is treated by administration to the subject of IGSF8 antagonist and an immuno-oncology agent, wherein the immuno-oncology agent is a CTLA-4 antagonist, such as an antagonistic CTLA-4 antibody. Suitable CTLA-4 antibodies include, for example, YERVOY (ipilimumab) or tremelimumab.

In one embodiment, a subject having a disease that may benefit from stimulation of the immune system, e.g., cancer or an infectious disease, is treated by administration to the subject of IGSF8 antagonist and an immuno-oncology agent, wherein the immuno-oncology agent is a PD-1 antagonist, such as an antagonistic PD-1 antibody. Suitable PD-1 antibodies include, for example, OPDIVO (nivolumab), KEYTRUDA (pembrolizumab), or MEDI-0680 (AMP-514; WO2012/145493). The immuno-oncology agent may also include pidilizumab (CT-011). Another approach to target the PD-1 receptor is the recombinant protein composed of the extracellular domain of PD-L2 (B7-DC) fused to the Fc portion of IgG1, called AMP-224.

In one embodiment, a subject having a disease that may benefit from stimulation of the immune system, e.g., cancer or an infectious disease, is treated by administration to the subject of IGSF8 antagonist and an immuno-oncology agent, wherein the immuno-oncology agent is a PD-L1 antagonist, such as an antagonistic PD-L1 antibody. Suitable PD-L1 antibodies include, for example, MPDL3280A (RG7446; WO2010/077634), durvalumab (MED14736), BMS-936559 (WO2007/005874), MSB0010718C (WO2013/79174) or rHigM12B7.

In one embodiment, a subject having a disease that may benefit from stimulation of the immune system, e.g., cancer or an infectious disease, is treated by administration to the subject of IGSF8 antagonist and an immuno-oncology agent, wherein the immuno-oncology agent is a LAG-3 antagonist, such as an antagonistic LAG-3 antibody. Suitable LAG3 antibodies include, for example, BMS-986016 (WO10/19570, WO 14/08218), or IMP-731 or IMP-321 (WO08/132601, WO09/44273).

In one embodiment, a subject having a disease that may benefit from stimulation of the immune system, e.g., cancer or an infectious disease, is treated by administration to the subject of IGSF8 antagonist and an immuno-oncology agent, wherein the immuno-oncology agent is a CD137 (4-1BB) agonist, such as an agonistic CD137 antibody. Suitable CD137 antibodies include, for example, urelumab or PF-05082566 (WO12/32433).

In one embodiment, a subject having a disease that may benefit from stimulation of the immune system, e.g., cancer or an infectious disease, is treated by administration to the subject of IGSF8 antagonist and an immuno-oncology agent, wherein the immuno-oncology agent is a GITR agonist, such as an agonistic GITR antibody. Suitable GITR antibodies include, for example, TRX-518 (WO06/105021, WO09/009116), MK-4166 (WO 11/028683) or a GITR antibody disclosed in WO2015/031667.

In one embodiment, a subject having a disease that may benefit from stimulation of the immune system, e.g., cancer or an infectious disease, is treated by administration to the subject of IGSF8 antagonist and an immuno-oncology agent, wherein the immuno-oncology agent is an OX40 agonist, such as an agonistic OX40 antibody. Suitable OX40 antibodies include, for example, MEDI-6383, MEDI-6469 or MOXR0916 (RG7888; WO06/029879).

In one embodiment, a subject having a disease that may benefit from stimulation of the immune system, e.g., cancer or an infectious disease, is treated by administration to the subject of IGSF8 antagonist and an immuno-oncology agent, wherein the immuno-oncology agent is a CD40 agonist, such as an agonistic CD40 antibody. In certain embodiments, the immuno-oncology agent is a CD40 antagonist, such as an antagonistic CD40 antibody. Suitable CD40 antibodies include, for example, lucatumumab (HCD122), dacetuzumab (SGN-40), CP-870,893 or Chi Lob 7/4.

In one embodiment, a subject having a disease that may benefit from stimulation of the immune system, e.g., cancer or an infectious disease, is treated by administration to the subject of IGSF8 antagonist and an immuno-oncology agent, wherein the immuno-oncology agent is a CD27 agonist, such as an agonistic CD27 antibody. Suitable CD27 antibodies include, for example, varlilumab (CDX-1127).

In one embodiment, a subject having a disease that may benefit from stimulation of the immune system, e.g., cancer or an infectious disease, is treated by administration to the subject of IGSF8 antagonist and an immuno-oncology agent, wherein the immuno-oncology agent is MGA271 (to B7H3) (WO1 1/109400).

In one embodiment, a subject having a disease that may benefit from stimulation of the immune system, e.g., cancer or an infectious disease, is treated by administration to the subject of IGSF8 antagonist and an immuno-oncology agent, wherein the immuno-oncology agent is a KIR antagonist, such as lirilumab.

In one embodiment, a subject having a disease that may benefit from stimulation of the immune system, e.g., cancer or an infectious disease, is treated by administration to the subject of IGSF8 antagonist and an immuno-oncology agent, wherein the immuno-oncology agent is an IDO antagonist. Suitable IDO antagonists include, for example, INCB-024360 (WO2006/122150, WO07/75598, WO08/36653, WO08/36642), indoximod, NLG-919 (WO09/73620, WO09/1156652, WO1 1/56652, WO 12/142237) or F001287.

In one embodiment, a subject having a disease that may benefit from stimulation of the immune system, e.g., cancer or an infectious disease, is treated by administration to the subject of IGSF8 antagonist and an immuno-oncology agent, wherein the immuno-oncology agent is a Toll-like receptor agonist, e.g., a TLR2/4 agonist (e.g., Bacillus Calmette-Guerin); a TLR7 agonist (e.g., Hiltonol or Imiquimod); a TLR7/8 agonist (e.g., Resiquimod); or a TLR9 agonist (e.g., CpG7909).

In one embodiment, a subject having a disease that may benefit from stimulation of the immune system, e.g., cancer or an infectious disease, is treated by administration to the subject of IGSF8 antagonist and an immuno-oncology agent, wherein, the immuno-oncology agent is a TGF-β inhibitor, e.g., GC1008, LY2157299, TEW7197 or IMC-TR1.

### 6. Exemplary IGSF8 Antagonists

In some embodiments, an IGSF8 antagonist is an IGSF8 antibody. In some embodiments, an IGSF8 antagonist for treating cancer may be a non-antibody protein, such as a soluble IGSF8 or a portion thereof (e.g., the ECD) that inhibits the interaction between IGSF8 and its ligand, optionally further comprising a fusion partner and in the form of a fusion molecule. The antagonist, in other embodiments, may also be a small molecule or small peptide.

### IGSF8 Antibodies

In some embodiments, antibodies that block binding of IGSF8 and its ligand are provided. In some embodiments, antibodies that inhibit IGSF8-mediated signaling are provided. In some such embodiments, the antibody is IGSF8 antibody. In some embodiments, the IGSF8 antibody binds to IGSF8 extracellular domain (ECD). In some embodiments, the IGSF8 antibody inhibits binding of IGSF8 to its ligand. In some embodiments, IGSF8 antibody inhibits IGSF8-mediated signaling. In some embodiments, IGSF8 antibody inhibits IGSF8-mediated signaling.

In some embodiments, IGSF8 antibody of the invention has a dissociation constant (K_{d}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸M to 10⁻¹³ M, e.g., from 10⁻⁹M to 10⁻¹³ M) for IGSF8, e.g., for humIGSF8. In certain embodiments, IGSF8 antibody has a dissociation constant (K_{d}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻¹³ M, e.g., from 10⁻⁹M to 10⁻¹³ M) for IGSF8, e.g., for humIGSF8.

In some embodiments, an IGSF8 antibody having any the characteristics provided herein inhibits at least 25%, 50%, 75%, 80%, 90% or 100% of the signaling of IGSF8.

In some embodiments, an IGSF8 antibody of the invention is any one of antibodies C1-C29, or C1-C12, as described in Example 7 (incorporated herein by reference).

In some embodiments, the invention provides an anti-IGSF8 monoclonal antibody or an antigen-binding fragment thereof specific for IGSF8, wherein the monoclonal antibody comprises: (1) a heavy chain variable region (HCVR), comprising HCVR CDR1-CDR3 sequences of any one of antibodies C1-C29, such as C1-C12; and, (2) a light chain variable region (LCVR), comprising LCVR CDR1-CDR3 sequences of said any one of antibodies C1-C29, such as C1-C12. In certain embodiment, the anti-IGSF8 monoclonal antibody or an antigen-binding fragment thereof has HCVR CDR1-CDR3 and LCVR CDR1-CDR3 of one of the antibodies C1-C29, such as any one of C1-C12.

In some embodiments, the monoclonal antibody or antigen-binding fragment thereof comprises: (a) the HCVR sequence of said any one of antibodies C1-C29, such as C1-C12; and/or, (b) the LCVR sequence of said any one of antibodies C1-C29, such as C1-C12. In certain embodiment, the anti-IGSF8 monoclonal antibody or an antigen-binding fragment thereof has HCVR and LCVR of one of the antibodies C1-C29, such as any one of C1-C12.

In some embodiments, the monoclonal antibody or antigen-binding fragment thereof is a human-mouse chimeric antibody, a humanized antibody, a human antibody, a CDR-grafted antibody, or a resurfaced antibody.

In some embodiments, the antigen-binding fragment thereof is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

In some embodiments, the monoclonal antibody or antigen-binding fragment thereof binds IGSF8 with a K_{d} of less than about 25 nM, 20 nM, 15 nM, 10 nM, 5 nM, 2 nM, or 1 nM.

In some embodiments, an antibody binds to IGSF8 from multiple species. For example, in some embodiments, an antibody binds to human IGSF8, and also binds to IGSF8 from at least one non-human mammal selected from mouse, rat, dog, guinea pig, and cynomolgus monkey.

In some embodiments, multispecific antibodies are provided. In some embodiments, bispecific antibodies are provided. Non-limiting exemplary bispecific antibodies include antibodies comprising a first arm comprising a heavy chain/light chain combination that binds a first antigen and a second arm comprising a heavy chain/light chain combination that binds a second antigen. A further non-limiting exemplary multispecific antibody is a dual variable domain antibody. In some embodiments, a bispecific antibody comprises a first arm that inhibits binding of IGSF8 and a second arm that stimulates T cells, e.g., by binding CD3. In some embodiments, the first arm binds IGSF8.

Another aspect of the invention provides a monoclonal antibody or an antigen-binding fragment thereof, which competes with the monoclonal antibody or antigen-binding fragment thereof of the invention described herein above.

### 7. Humanized Antibodies

In some embodiments, the IGSF8 antibody is a humanized antibody. Humanized antibodies are useful as therapeutic molecules because humanized antibodies reduce or eliminate the human immune response to non-human antibodies (such as the human anti-mouse antibody (HAMA) response), which can result in an immune response to an antibody therapeutic, and decreased effectiveness of the therapeutic.

An antibody may be humanized by any standard method. Non-limiting exemplary methods of humanization include methods described, e.g., in U.S. Pat. Nos. 5,530,101; 5,585,089; 5,693,761; 5,693,762; 6,180,370; Jones et al., Nature 321:522-525 (1986); Riechmann et al, Nature 332: 323-27 (1988); Verhoeyen et al, Science 239: 1534-36 (1988); and U.S. Publication No. US 2009/0136500. All incorporated by reference.

A humanized antibody is an antibody in which at least one amino acid in a framework region of a non-human variable region has been replaced with the amino acid from the corresponding location in a human framework region. In some embodiments, at least two, at least three, at least four, at least five, at least six, at least seven, at least eight, at least nine, at least 10, at least 11, at least 12, at least 15, or at least 20 amino acids in the framework regions of a non-human variable region are replaced with an amino acid from one or more corresponding locations in one or more human framework regions.

In some embodiments, some of the corresponding human amino acids used for substitution are from the framework regions of different human immunoglobulin genes. That is, in some such embodiments, one or more of the non-human amino acids may be replaced with corresponding amino acids from a human framework region of a first human antibody or encoded by a first human immunoglobulin gene, one or more of the non-human amino acids may be replaced with corresponding amino acids from a human framework region of a second human antibody or encoded by a second human immunoglobulin gene, one or more of the non-human amino acids may be replaced with corresponding amino acids from a human framework region of a third human antibody or encoded by a third human immunoglobulin gene, etc. Further, in some embodiments, all of the corresponding human amino acids being used for substitution in a single framework region, for example, FR2, need not be from the same human framework. In some embodiments, however, all of the corresponding human amino acids being used for substitution are from the same human antibody or encoded by the same human immunoglobulin gene.

In some embodiments, an antibody is humanized by replacing one or more entire framework regions with corresponding human framework regions. In some embodiments, a human framework region is selected that has the highest level of homology to the non-human framework region being replaced. In some embodiments, such a humanized antibody is a CDR-grafted antibody.

In some embodiments, following CDR-grafting, one or more framework amino acids are changed back to the corresponding amino acid in a mouse framework region. Such “back mutations” are made, in some embodiments, to retain one or more mouse framework amino acids that appear to contribute to the structure of one or more of the CDRs and/or that may be involved in antigen contacts and/or appear to be involved in the overall structural integrity of the antibody. In some embodiments, ten or fewer, nine or fewer, eight or fewer, seven or fewer, six or fewer, five or fewer, four or fewer, three or fewer, two or fewer, one, or zero back mutations are made to the framework regions of an antibody following CDR grafting.

In some embodiments, a humanized antibody also comprises a human heavy chain constant region and/or a human light chain constant region.

### 8. Chimeric Antibodies

In some embodiments, the IGSF8 antibody is a chimeric antibody. In some embodiments, the IGSF8 antibody comprises at least one non-human variable region and at least one human constant region. In some such embodiments, all of the variable regions of the IGSF8 antibody are non-human variable regions, and all of the constant regions of the IGSF8 antibody are human constant regions. In some embodiments, one or more variable regions of a chimeric antibody are mouse variable regions. The human constant region of a chimeric antibody need not be of the same isotype as the non-human constant region, if any, it replaces. Chimeric antibodies are discussed, e.g., in U.S. Pat. No. 4,816,567; and Morrison et al., Proc. Natl. Acad. Sci. USA 81: 6851-55 (1984).

### 9. Human Antibodies

In some embodiments, the IGSF8 antibody is a human antibody. Human antibodies can be made by any suitable method. Non-limiting exemplary methods include making human antibodies in transgenic mice that comprise human immunoglobulin loci. See, e.g., Jakobovits et al., Proc. Natl. Acad. Sci. USA 90: 2551-55 (1993); Jakobovits et al, Nature 362: 255-8 (1993); onberg et al, Nature 368: 856-9 (1994); and U.S. Pat. Nos. 5,545,807; 6,713,610; 6,673,986; 6,162,963; 5,545,807; 6,300,129; 6,255,458; 5,877,397; 5,874,299;

Non-limiting exemplary methods also include making human antibodies using phage display libraries. See, e.g., Hoogenboom et al., J. Mol. Biol. 227: 381-8 (1992); Marks et al, J. Mol. Biol. 222: 581-97 (1991); and PCT Publication No. WO 99/10494.

Human Antibody Constant Regions

In some embodiments, a humanized, chimeric, or human antibody described herein comprises one or more human constant regions. In some embodiments, the human heavy chain constant region is of an isotype selected from IgA, IgG, and IgD. In some embodiments, the human light chain constant region is of an isotype selected from K and λ. In some embodiments, an antibody described herein comprises a human IgG constant region, for example, human IgG1, IgG2, IgG3, or IgG4. In some embodiments, an antibody or Fc fusion partner comprises a C237S mutation, for example, in an IgG1 constant region. In some embodiments, an antibody described herein comprises a human IgG2 heavy chain constant region. In some such embodiments, the IgG2 constant region comprises a P331S mutation, as described in U.S. Pat. No. 6,900,292. In some embodiments, an antibody described herein comprises a human IgG4 heavy chain constant region. In some such embodiments, an antibody described herein comprises an S241P mutation in the human IgG4 constant region. See, e.g., Angal et al. Mol. Immunol. 30(1):105-108 (1993). In some embodiments, an antibody described herein comprises a human IgG4 constant region and a human κ light chain.

The choice of heavy chain constant region can determine whether or not an antibody will have effector function in vivo. Such effector function, in some embodiments, includes antibody-dependent cell-mediated cytotoxicity (ADCC) and/or complement-dependent cytotoxicity (CDC), and can result in killing of the cell to which the antibody is bound. Typically, antibodies comprising human IgG1 or IgG3 heavy chains have effector function.

In some embodiments, effector function is not desirable. For example, in some embodiments, effector function may not be desirable in treatments of inflammatory conditions and/or autoimmune disorders. In some such embodiments, a human IgG4 or IgG2 heavy chain constant region is selected or engineered. In some embodiments, an IgG4 constant region comprises an S241P mutation.

Any of the antibodies described herein may be purified by any suitable method. Such methods include, but are not limited to, the use of affinity matrices or hydrophobic interaction chromatography. Suitable affinity ligands include the antigen and/or epitope to which the antibody binds, and ligands that bind antibody constant regions. For example, a Protein A, Protein G, Protein A/G, or an antibody affinity column may be used to bind the constant region and to purify an antibody.

In some embodiments, hydrophobic interactive chromatography (HIC), for example, a butyl or phenyl column, is also used for purifying some polypeptides. Many methods of purifying polypeptides are known in the art.

Alternatively, in some embodiments, an antibody described herein is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al, Biotechnol. Adv. 21: 695-713 (2003).

### 10. Antibody Properties

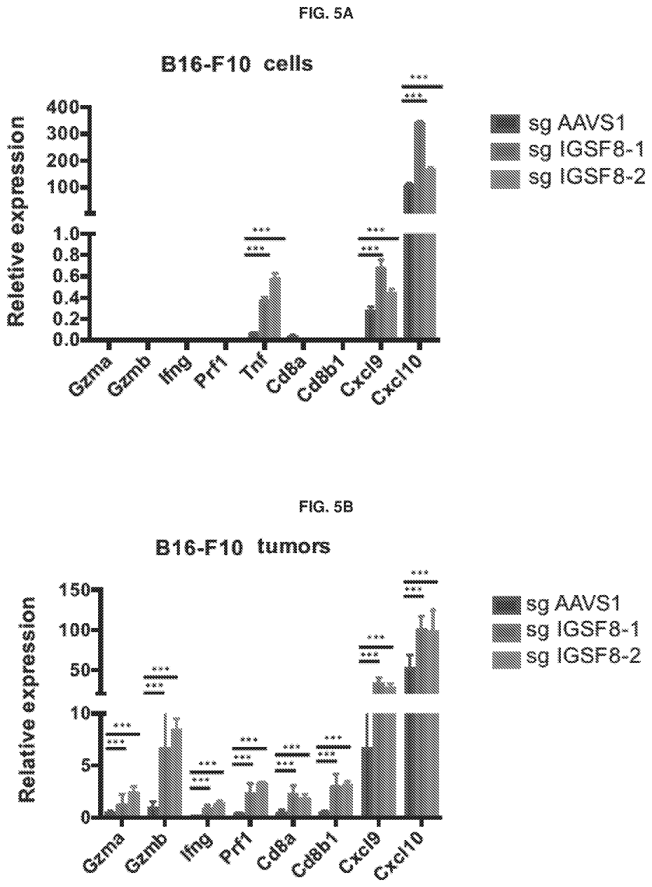

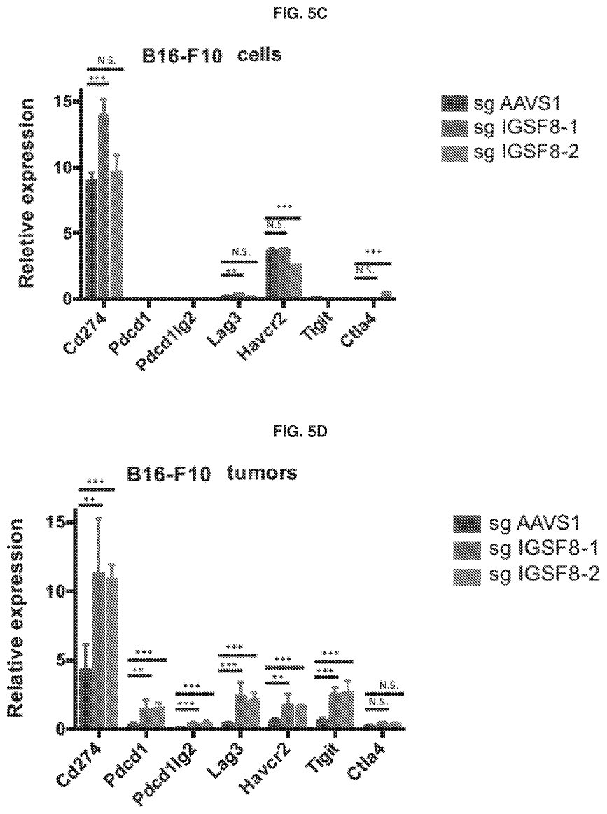

In some embodiments, the subject IGSF8 antibody binds to IGSF8 and inhibits IGSF8-mediated signaling, such as up- or down-regulation of the downstream genes as indicated in FIGS. 4, and 5A-5D. In some embodiments, IGSF8 antibody binds to IGSF8 with a binding affinity (K_{D}) or EC50 value of less than 50 nM, less than 20 nM, less than 10 nM, or less than 1 nM. In some embodiments, the extent of binding of IGSF8 antibody to an unrelated, non-IGSF8 protein is less than about 10% of the binding of the antibody to IGSF8 as measured, e.g., by a radioimmunoassay (RIA). In some embodiments, IGSF8 antibody binds to an epitope of IGSF8 that is conserved among IGSF8 from different species. In some embodiments, IGSF8 antibody binds to the same epitope as a human or humanized IGSF8 antibody that binds humIGSF8.

In some embodiments, the IGSF8 antibody is conjugated to a label, which is a moiety that facilitates detection of the antibody and/or facilitates detection of a molecule to which the antibody binds. Nonlimiting exemplary labels include, but are not limited to, radioisotopes, fluorescent groups, enzymatic groups, chemiluminescent groups, biotin, epitope tags, metal-binding tags, etc. One skilled in the art can select a suitable label according to the intended application.

In some embodiments, a label is conjugated to an antibody using chemical methods in vitro. Nonlimiting exemplary chemical methods of conjugation are known in the art, and include services, methods and/or reagents commercially available from, e.g., Thermo Scientific Life Science Research Produces (formerly Pierce; Rockford, Ill.), Prozyme (Hayward, Calif.), SACRI Antibody Services (Calgary, Canada), AbD Serotec (Raleigh, N.C.), etc. In some embodiments, when a label is a polypeptide, the label can be expressed from the same expression vector with at least one antibody chain to produce a polypeptide comprising the label fused to an antibody chain.

### 11. IGSF8 ECDs, Fusions, and Small Peptides

In some embodiments, the IGSF8 antagonist is an IGSF8 polypeptide, such as a full-length IGSF8, or a fragment thereof that inhibits binding of IGSF8 to its ligand. In some embodiments, the IGSF8 antagonist is an IGSF8 extracellular domain (ECD). In some embodiments, the IGSF8 antagonist is a full-length IGSF8 ECD. In some embodiments, the IGSF8 ECD is an IGSF8 ECD fragment, for example, comprising at least 80%, at least 85%, at least 90%, or at least 95% of the full length IGSF8 ECD amino acid sequence from which it is derived. In some embodiments, the IGSF8 ECD is an IGSF8 ECD variant, for example, comprising at least 80%, at least 85%, at least 90%, at least 92%, at least 95%, at least 97%, at least 98%, or at least 99% sequence identity with the full length IGSF8 ECD from which it is derived. In other embodiments, the IGSF8 ECD is from a non-human IGSF8 ECD and may be either full length, a fragment, or a variant.

In some embodiments, the IGSF8 or IGSF8 fragment is combined with at least one fusion partner. Thus, in some such embodiments, the IGSF8 antagonist may comprise a full length IGSF8 ECD and at least one fusion partner to form a IGSF8 ECD fusion molecule. In some embodiments, the IGSF8 ECD portion of the fusion molecule comprises a IGSF8 ECD fragment, for example, comprising at least 80%, at least 85%, at least 90%, or at least 95% of the full length IGSF8 ECD amino acid sequence from which it is derived. In some embodiments, the IGSF8 ECD portion of the fusion molecule is a IGSF8 ECD variant, for example, comprising at least 80%, at least 85%, at least 90%, at least 92%, at least 95%, at least 97%, at least 98%, or at least 99% sequence identity with the full length IGSF8 ECD from which it is derived. In other embodiments, the IGSF8 component is from a non-human IGSF8 ECD and may be full length, a fragment, or a variant. In any of the fusion molecule embodiments above, the fusion partner may comprise an immunoglobulin Fc molecule, for example, a human Fc molecule, or in some embodiments. In other embodiments, the fusion partner may be a different molecule such as albumin or polyethylene glycol (PEG). In some embodiments, more than one fusion partner may be attached to the IGSF8 ECD. In some embodiments, the fusion partner (or partners) is attached at the C-terminal of the ECD, while other attachments are also possible such as on an amino acid side-chain or at the N-terminus. The attachment of a fusion partner to a IGSF8 ECD may be direct (i.e. by a covalent bond) or indirect through a linker. A linker may comprise, for example, at least one intervening amino acid or some other chemical moiety serving to link the fusion partner to the ECD either covalently or noncovalently.

In any of the above embodiments, the IGSF8 polypeptide may either include a signal sequence or be in a mature form, i.e., not including a signal sequence. The signal sequence may be from a native IGSF8 molecule or it may be a signal sequence from a different protein, for example one chosen to enhance expression of the IGSF8 polypeptide in cell culture.

In some embodiments a IGSF8 ECD may comprise the following sequence:

(SEQ ID NO: 468)
REVLVPEGPLYRVAGTAVSISCNVTGYEGPAQQNFEWFLYRPEAPDTALG
IVSTKDTQFSYAVFKSRWAGEVQVQRLQGDAWLKIARLQAQDAGIYECHT
PSTDTRYLGSYSGKVELRVLPDVLQVSAAPPGPRGRQAPTSPPRMTVHEG
QELALGCLARTSTQKHTHLAVSFGRSVPEAPVGRSTLQEWGIRSDLAVEA
GAPYAERLAAGELRLGKEGTDRYRMWGGAQAGDAGTYHCTAAEWIQDPDG
SWAQIAEKRAVLAHVDVQTLSSQLAVTVGPGERRIGPGEPLELLCNVSGA
LPPAGRHAAYSVGWEMAPAGAPGPGRLVAQLDTEGVGSLGPGYEGRHIAM
EKVASRTYRLRLEAARPGDAGTYRCLAKAYVRGSGTRLREAASARSRPLP
VHVREEGWLEAVAWLAGGTVYRGETASLLCNISVRGGPPGLRLAASWWVE
RPEDGELSSVPAQLVGGVGQDGVAELGVRPGGGPVSVELVGPRSHRLRLH
SLGPEDEGVYHCAPSAWVQHADYSWYQAGSARSGPVTVYPYMHALDT

In any of the above cases, a IGSF8 ECD may be part of a fusion molecule such that the above amino acid sequence may be joined to a fusion partner either directly or via a linker, such as an Fc, albumin, or PEG. For example, in some embodiments in which the antagonist is a IGSF8 ECD fusion molecule, the fusion molecule may comprise one of the above sequences plus an immunoglobulin Fc sequences, or an Fc from human IgG1. An IGSF8 ECD Fc fusion molecule may be formed by a direct attachment of the IGSF8 ECD amino acid sequence to the Fc amino acid sequence or via a linker (either an intervening amino acid or amino acid sequence or another chemical moiety).

In some embodiments, the IGSF8 antagonist may be a small molecule or a peptide, e.g., a small peptide. In some embodiments, the IGSF8 antagonist may be a small peptide comprising an amino acid sequence of an IGSF8 ECD fragment. In some embodiments, the IGSF8 antagonist is a small peptide having, e.g., from 3 to 20, e.g., 3 to 15 or 3 to 10 amino acids, which peptide may be linear or circular, with a sequence comprising an IGSF8 fragment, an IGSF8 ECD fragment, or a variant of an IGSF8 fragment, or IGSF8 ECD fragment. Such a variant of a IGSF8 may have, for example, at least 95%, at least 97%, at least 99% sequence identity to the native fragment sequence from which it is derived.

In certain embodiments, any of the polypeptides of the invention, including antibodies antigen-binding portion thereof, IGSF8 polypeptide and ECD thereof, may have a heterologous signal peptide when synthesized. In order for some secreted proteins to express and secrete in large quantities, a signal peptide from a heterologous protein may be desirable. Employing heterologous signal peptides may be advantageous in that a resulting mature polypeptide may remain unaltered as the signal peptide is removed in the ER during the secretion process. The addition of a heterologous signal peptide may be required to express and secrete some proteins.

Non-limiting exemplary signal peptide sequences are described, e.g., in the online Signal Peptide Database maintained by the Department of Biochemistry, National University of Singapore. See Choo et al, BMC Bioinformatics, 6: 249 (2005); and PCT Publication No. WO 2006/081430.

### 12. Co-Translational and Post-Translational Modifications

In some embodiments, a polypeptide such as IGSF8 or an IGSF8 ECD is differentially modified during or after translation, for example by glycosylation, sialylation, acetylation, phosphorylation, amidation, derivatization by known protecting/blocking groups, proteolytic cleavage, or linkage to an antibody molecule or other cellular ligand. Any of numerous chemical modifications may be carried out by known techniques, including, but not limited to, specific chemical cleavage by cyanogen bromide, trypsin, chymotrypsin, papain, V8 protease; NABH4; acetylation; formylation; oxidation; reduction; and/or metabolic synthesis in the presence of tunicamycin.

Additional post-translational modifications encompassed by the invention include, for example, N-linked or O-linked carbohydrate chains; processing of N-terminal or C-terminal ends; attachment of chemical moieties to the amino acid backbone; chemical modifications of N-linked or O-linked carbohydrate chains; and addition or deletion of an N-terminal methionine residue as a result of prokaryotic host cell expression.

### 13. Nucleic Acid Molecules Encoding IGSF8 Antagonists

The invention also provides nucleic acid molecules comprising polynucleotides that encode one or more chains of an antibody described herein, such as IGSF8 antibody. In some embodiments, a nucleic acid molecule comprises a polynucleotide that encodes a heavy chain or a light chain of an antibody described herein. In some embodiments, a nucleic acid molecule comprises both a polynucleotide that encodes a heavy chain and a polynucleotide that encodes a light chain, of an antibody described herein. In some embodiments, a first nucleic acid molecule comprises a first polynucleotide that encodes a heavy chain and a second nucleic acid molecule comprises a second polynucleotide that encodes a light chain.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an antibody described herein comprises a nucleotide sequence that encodes a leader sequence, which, when translated, is located at the N-terminus of the heavy chain or light chain. As discussed above, the leader sequence may be the native heavy or light chain leader sequence, or may be another heterologous leader sequence.

Nucleic acids encoding other IGSF8 antagonists are also provided, such as fragments or variants of IGSF8 including IGSF8 ECD molecules, or IGSF8 ECD fusion molecules and including fragments or variants of VISTA including VISTA ECD molecules or VISTA ECD fusion molecules. Nucleic acid molecules may be constructed using recombinant DNA techniques conventional in the art. In some embodiments, a nucleic acid molecule is an expression vector that is suitable for expression in a selected host cell.

### 14. Vectors

Vectors comprising polynucleotides that encode heavy chains and/or light chains of the antibodies described herein are provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, e.g., in Running Deer et al., Biotechnol. Prog. 20:880-889 (2004). In some embodiments, a vector is chosen for in vivo expression of IGSF8 antagonist in animals, including humans. In some such embodiments, expression of the polypeptide or polypeptides is under the control of a promoter or promoters that function in a tissue-specific manner. For example, liver-specific promoters are described, e.g., in PCT Publication No. WO 2006/076288.

### 15. Host Cells

In various embodiments, heavy chains and/or light chains of the antibodies described herein may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO—S and DG44 cells; PER.C6® cells (Crucell); and NSO cells. In some embodiments, heavy chains and/or light chains of the antibodies described herein may be expressed in yeast. See, e.g., U.S. Publication No. US 2006/0270045 A1. In some embodiments, a particular eukaryotic host cell is selected based on its ability to make desired post-translational modifications to the heavy chains and/or light chains of IGSF8 antibody. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc., Nonlimiting exemplary methods are described, e.g., in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

In some embodiments, one or more polypeptides may be produced in vivo in an animal that has been engineered or transfected with one or more nucleic acid molecules encoding the polypeptides, according to any suitable method.

### EXAMPLES

### Example 1 Loss of IGSF8 in Colo205 Cancer Cells Enhances Natural Killer (NK) Cell Cytotoxicity Against Colo205 Cells

This experiment demonstrates that IGSF8 activity/expression negatively regulates NK cell cytotoxicity towards cancer cells (e.g., Colo205 colorectal cancer cells), and loss of IGSF8 activity/expression enhances NK cell cytotoxicity.

A genome-wide co-culture screen using NK cell and Colo205 cancer cells were conducted to determine which gene(s) are required or are essential for Colo205 cancer cells to evade killing by NK cells. In particular, Colo205 tumor cells were transduced with a whole-genome guide RNA (gRNA) Cas9 library and then subjected to two successive rounds of overnight co-culture with primary human NK cells which exhibited a typical activated phenotype. The resulting population of cells were sequenced to identify depleted gRNA that sensitized tumor cells to killing by NK cells. Model-based Analysis of Genome-wide CRISPR/Cas9 Knockout (MAGeCK) software was subsequently used to count the reads and perform gene/gRNA fold change, selection score and statistical analyses between treated and untreated (control) samples.

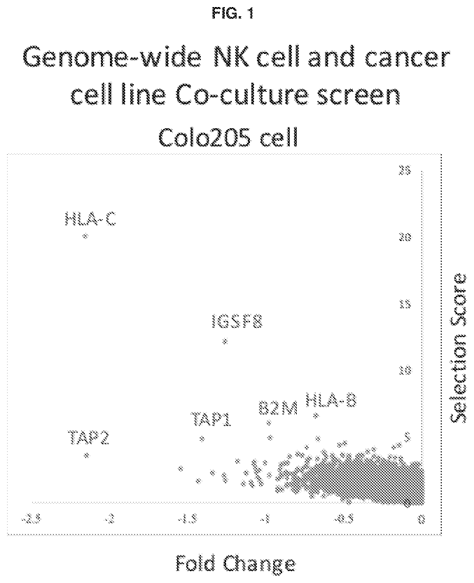

A volcano dot plot encompassing selection score and gRNA fold change was generated for each gene tested in the assay, showing the top depleted genes after co-culturing with NK cells. The genes associated with antigen presentation (such as HLA-C, Tap1, Tap2, and B2m), when depleted, were found to render the tumor cells most sensitive to killing by NK cells. Additionally, IGSF8 was one of the two top hits, the loss of which activity/expression in Colo205 cell enhanced NK cell cytotoxicity. The results were summarized in FIG. 1.

### Example 2 IGSF8 Reduced Viability of Primary Natural Killer Cells and Primary T Cells from Healthy Donors

To further demonstrate the negative impact of IGSF8 on NK cell activity, increasing concentrations of recombinant human IGSF8 tagged by a human Fc region (IGSF8-hFc) was incubated with primary human NK cells isolated from two healthy donors, and the viability of these primary NK cells over IGSF8-hFc concentrations (dose response curve) was determined.

The primary NK or T cells were isolated from healthy donors' peripheral blood mononuclear cells (PBMCs) using commercial negative/positive isolation kits (StemCell Technologies, Inc.). NK or T cells were cultured in RPMI medium supplemented with 10% Fetal Bovine Serum (FBS), penicillin/streptomycin, L-glutamine, non-essential amino acids, sodium pyruvate, HEPES, 2-Mercaptoethanol and recombinant human IL-2 (1,000 IU/mL), and were incubated at 37° C. with 5% CO₂. T cells were activated by Anti-CD3 and CD28 beads once a week.

The primary NK or T cells were then seeded in 96-well plates (3,000 cells per well) and cultured 18 to 24 hours before adding the IGSF8-hFc fusion protein or human Fc protein as negative control. Cell viability was determined by Cell Counting Kit 8 (CCK8) method with three biological replicates after 72 hours.

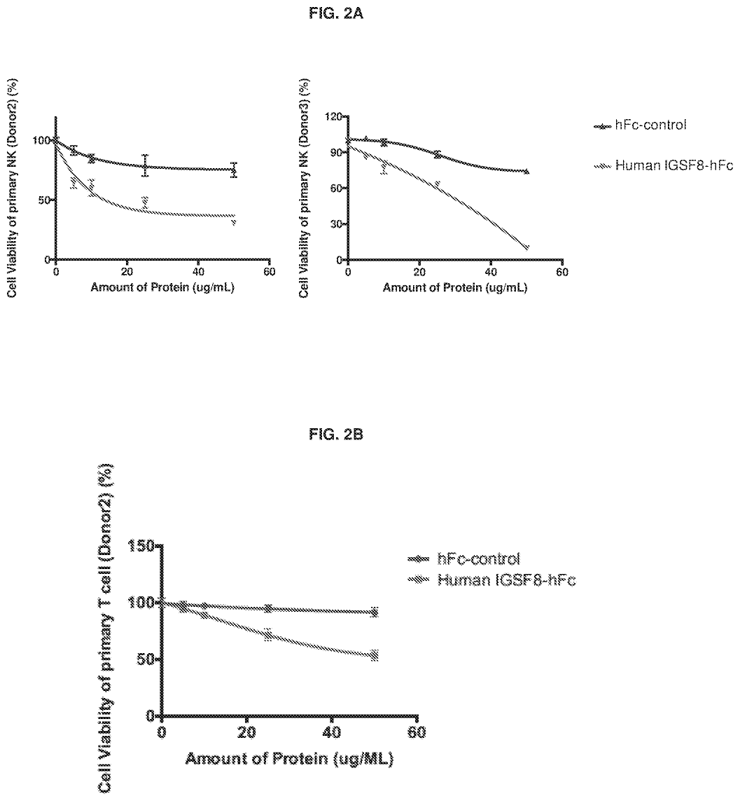

Data in FIG. 2A shows that NK cell viability was reduced in vitro as concentration of IGSF8-hFc increased. Meanwhile, a human Fc used as a control in the same assay did not substantially affect NK cell viability. This data is consistent with the observation in Example 1 that the presence of IGSF8 on Colo205 cancer cells inhibited NK cell function, possibly at least partially through reducing NK cell viability.

Similar results were also obtained for primary T lymphocytes isolated from Donor 2. See FIG. 2B.

These data showed that IGSF8 reduced viability of both primary NK cells and primary T cells in vitro, suggesting a mechanism by which antagonizing IGSF8 activity can be used to restore or promote NK/T cell activity.

### Example 3 CRISPR/Cas9-Mediated IGSF8 Knock-Out in B16-F10 Tumor Cells Retards Tumor Growth In Vivo in Syngeneic Tumor Model

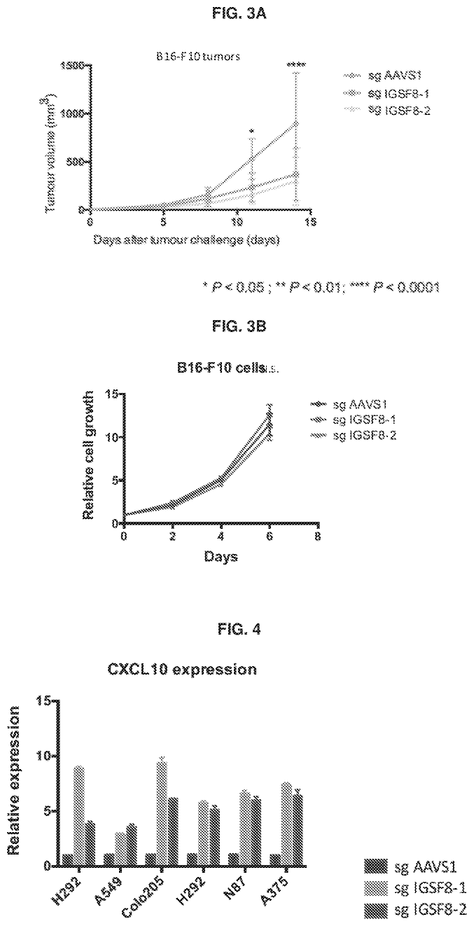

To further demonstrate the negative impact of tumor-expressed IGSF8 on the host immune system, B16-F10 melanoma cells with or without IGSF8 function/expression (IGSF8 null) were compared in their ability to grow as syngeneic tumors in wild-type (WT) mice. The IGSF8 gene was deleted/inactivated by the CRISPR/Cas9-mediated gene editing using IGSF8-specific single guide RNA (sgRNA) sequences. Two separate lines of IGSF8-inactivated B16-F10 cancer cell lines were established, namely sg IGSF8-1 and sg IGSF8-2, with different regions of IGSF8 being targeted. Down-regulation of IGSF8 expression was verified by flow cytometry (data not shown). As a negative control, the adeno associated virus integration sequence AAVS1 was also similarly deleted/inactivated by CRISPR/Cas9-mediated gene editing in B16-F10 cells (sg AAVS1). Then one million each of unaltered B16-F10 cancer cells, sg IGSF8-1 cells, sg IGSF8-2 cells, and sg AAVS1 cells, respectively, were implanted into C57BL/6 mice (8 mice per group) at Day 0, and tumor volumes in each mouse was measured and calculated according to standard methods over 2 weeks. The results were averaged for each group with standard deviation, and plotted in FIG. 3A.

It is apparent that the absence of IGSF8 expression/function significantly retarded tumor growth as early as Day 11 (p<0.05), and the difference in tumor volume was significant at Day 14 (p<0.0001). This in vivo result is consistent with the previous observation that IGSF8 reduced NK and T cell viability in vitro.

Interestingly, the presence or absence of IGSF8 was apparently not required for tumor growth per se. Relative tumor cell growth rates over a course of 6 days, as measured in vitro for each of the above test cell lines, were essentially indistinguishable (see FIG. 3B).

This result is also consistent with the observation that the average essential score of IGSF8, in a genome-wide CRISPR screen based on 625 types of cancer cell lines (Data downloaded from DepMap Portal), was just slightly negative and very close to 0 (about −0.05) (data not shown), suggesting that IGSF8 plays a very minor (if any) direct role in cell growth. In contrast, prototypical oncogenes such as myc, and cell cycle genes such as CDK1, were both well below −1.0, while tumor suppressor gene Tp53 has a +0.2 average essential score (data not shown).

Together, these data strongly suggest that the absence of IGSF8 on tumor cells retarded tumor cell growth in vivo, not through reducing the growth rate of the tumor cells per se, but likely through negatively affecting (e.g., inhibiting) the host immune system.

### Example 4 TNFα Signaling Pathway is Negatively Regulated by IGSF8

To identify the mechanism by which loss of IGSF8 in tumor cells allows the tumor cells to escape immune surveillance, RNA-sequencing was performed for both IGSF8-null and AAVS1-control B16-F10 melanoma cells as described in Example 3.

Importantly, it was found that depletion of IGSF8 in B16-F10 cells activated TNFα signaling pathway, and increased gene expressions of many immune-related cytokines (especially, CXCL10 and CXCL9, see FIGS. 5A-5B). CXCL10 is a small cytokine belonging to the CXC chemokine family, which plays role to induce chemotaxis, promote differentiation, and multiplication of leukocytes, and cause tissue extravasation. CXCL10 is secreted by several cell types in response to IFN-γ.

As CXCL9 and CXCL10 were known to regulate immune cell migration, differentiation, and activation, leading to tumor suppression (Tokunaga et al., Cancer Treat Rev. 63:40-47, 2018), the effect of IGSF8 on CXCL10 expression in other human cancer cells was examined.

Specifically, IGSF8 was knocked out in six different human cancer cell lines by CRISPR/Cas9, and RNA-sequencing was performed for these IGSF8-null and AAVS1-control human cancer cells. FIG. 4 shows that relative expression of CXCL10 in the various tested tumor cell lines were increased, sometimes dramatically increased by almost 10-fold, in IGSF8 null cancer cells compared to the counterpart cancer cell lines with intact IGSF8. The tested cancer cell lines included: H292 (NCI-H292) is a human mucoepidermoid pulmonary carcinoma cell line; A549 is a human lung carcinoma cell line; Colo205 is a Dukes' type D, colorectal adenocarcinoma cell line; N87 is a human gastric carcinoma cell line; and A375 is a another human melanoma cell line.

These data suggest that IGSF8 may be a universal negative regulator of CXCL10 expression in various cancers, and deletion or inactivation of IGSF8 promotes CXCL10 expression.

### Example 5 Loss of IGSF8 Reprogramed the Tumor Microenvironment (TME) to Improve NK and T Cell Activities

To identify the mechanism by which inactivation of IGSF8 in B16-F10 tumors significantly decreased tumor growth (see FIG. 3A), IGSF8-null and AAVS1-control B16-F10 cells were subcutaneously inoculated into C57BL6 mice. When the tumors grew to about 1 to 2 mm³, the tumors were isolated, and RNA-sequencing was performed on isolated tumors.

It was found that the genes (Gzmb, Prfl, etc.) representing the immune cytolytic activity (CYT) of tumors were significantly up-regulated in IGSF8-null tumors (FIG. 5B), but not in IGSF8-null cells (FIG. 5A). Moreover, CD8 gene (CD8a and CD8b) expression in IGSF8-null tumors (but not in IGSF8 null cells, FIG. 5A) were also dramatically increased (FIG. 5B), indicating more CD8⁺ T cell infiltration into IGSF8-null tumors.

These data suggest that depletion of IGSF8 in B16-F10 tumors reprogramed the Tumor Microenvironment (TME) to improve immune cytolytic activity in vivo for tumor suppression, possibly by increasing CD8⁺ T cell infiltration.

More importantly, loss of IGSF8 increased the expression of well established IO targets (PDCD1, CD274, LAG3, TIM3 or TIGIT) (FIG. 5D), indicating that combining IGSF8 antagonists with antagonists of PDCD1, CD274, Lag3, TIM3 or TIGIT in a combination therapy is effective for cancer treatment. See below.

### Example 6 IGSF8 was Overexpressed in Many Cancer Types and Resulted in Worse Clinical Outcome

This example demonstrates that IGSF8 is overexpressed by a number of cancer cells, possibly as a mechanism to evade host immune response.

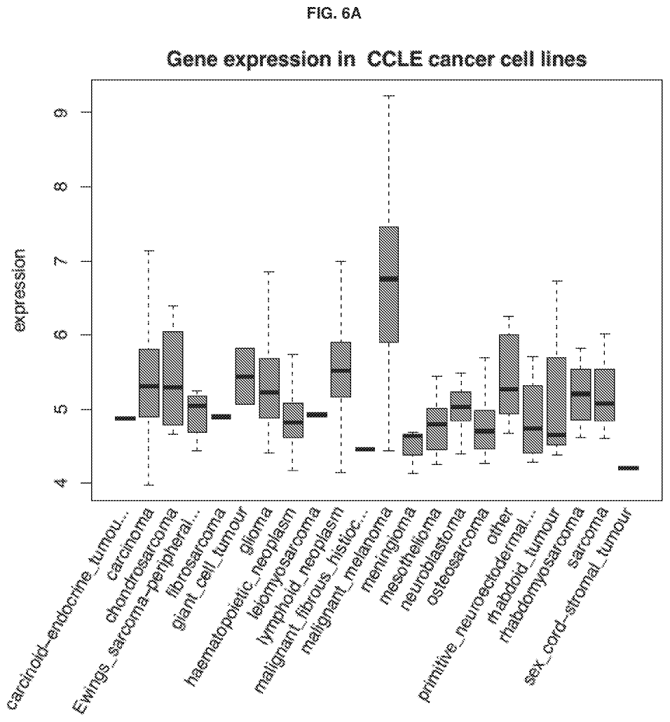

FIG. 6A shows gene expression of IGSF8 in a number of human cancer cell lines based on data from Broad Institute Cancer Cell Line Encyclopedia (CCLE). Top 30 cancer cell lines with the highest IGSF8 expression in the CCLE dataset are listed below.

In addition, based on analysis of The Cancer Genome Atlas (TCGA) Datasets, IGSF8 was found to be significantly overexpressed in many types of cancers: BLCA: Bladder Cancer, BRCA: Breast Cancer, HNSC: Head-Neck Squamous Cell Carcinoma, LUAD: Lung Adenocarcinoma, LUSC: Lung Squamous Cell Carcinoma, PRAD: Prostate Adenocarcinoma, SKCM: Skin Cutaneous Melanoma, THCA: Thyroid Cancer, UCEC: Uterine Corpus Endometrial Carcinoma, READ: Rectum Adenocarcinoma, COAD: Colon Adenocarcinoma (FIG. 6B).

RSEM (RNA-Seq by Expectation-Maximization)

| | IGSF8 expression |
|---|---|
| Cell line | (log2(RSEM) |
| MALME3M SKIN | 9.226186 |
| HS936T_SKIN | 8.806057 |
| IGR37_SKIN | 8.626165 |
| K029AX_SKIN | 8.458715 |
| COLO679_SKIN | 8.448694 |
| DU4475 BREAST | 8.439735 |
| MELHO_SKIN | 8.34886 |
| COLO741_SKIN | 8.26553 |
| TT THYROID | 8.093418 |
| SKMEL2_SKIN | 8.006397 |
| SKMEL5_SKIN | 8.005364 |
| G361_SKIN | 7.911904 |
| NCIH520_LUNG | 7.905627 |
| C32 SKIN | 7.901319 |
| COLO829_SKIN | 7.896537 |
| MHHNB11 AUTONOMIC GANGLIA | 7.838727 |
| UACC257_SKIN | 7.74993 |
| H [symbol] 939T_SKIN | 7.722027 |
| UBLC1 URINARY TRACT | 7.69668 |
| KURAMOCHI_OVARY | 7.67295 |
| OE19 OESOPHAGUS | 7.598727 |
| UACC62_SKIN | 7.554536 |
| CAL148_BREAST | 7.51395 |
| HCC1419 BREAST | 7.477927 |
| JHH2_LIVER | 7.471425 |
| H [symbol] 944T_SKIN | 7.460963 |
| SKMEL30_SKIN | 7.453764 |
| AU565_BREAST | 7.443264 |
| SKMEL24 SKIN | 7.425736 |
| BT483_BREAST | 7.419773 |

[symbol]

indicates data missing or illegible when filed

The clinical relevancy of IGSF8 expression was also demonstrated by data based on The Cancer Genome Atlas (TCGA). Specifically, FIG. 6C shows that higher expression of IGSF8 is associated with worse clinical outcome in different cancer types. For example, in melanoma, the 13 patients with high IGSF8 expression (“Top”) had a much worse survival curve than that for the 304 patients with lower IGSF8 expression (“Bottom”). The difference is statistically significant (p<0.0018).

The same has been observed in cervical cancer, LUAD (lung adenocarcinoma), lymphoma (including diffused large B cell lymphoma or DLBCL), LUSC (Lung Squamous Cell Carcinoma), READ (Rectum Adenocarcinoma), COAD (colon adenocarcinoma), and leukemia (including CLL).

Thus it is expected that IGSF8 antagonists of the invention, such as anti-IGSF8 antibodies or antigen-binding fragments thereof, are able to treat cancers with IGSF8 overexpression, such as the cancers listed in the table above and those in FIGS. 6A-6C.

### Example 7 Anti-IGSF8 Antibodies Exhibit Nanomolar (nM) Affinity for IGSF8 Extracellular Domain (ED)

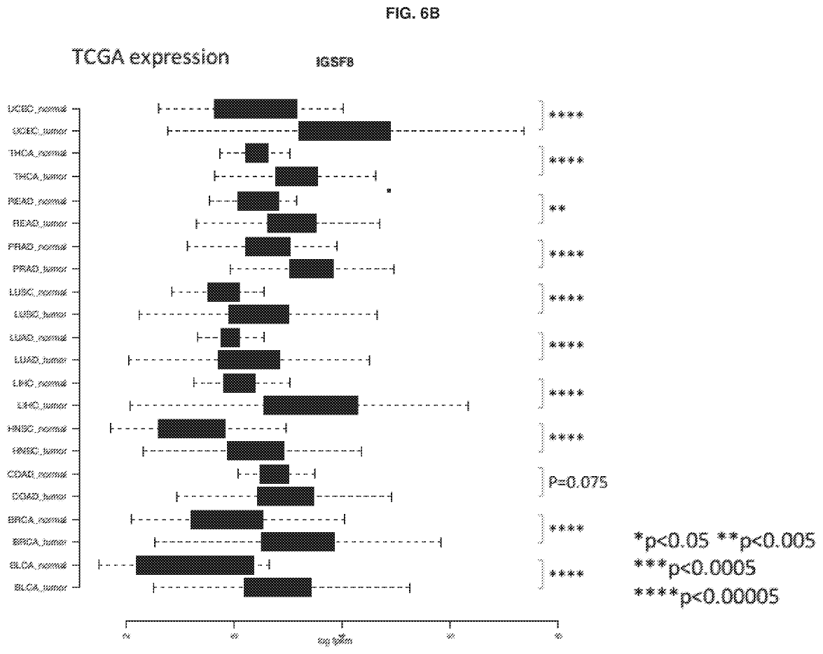

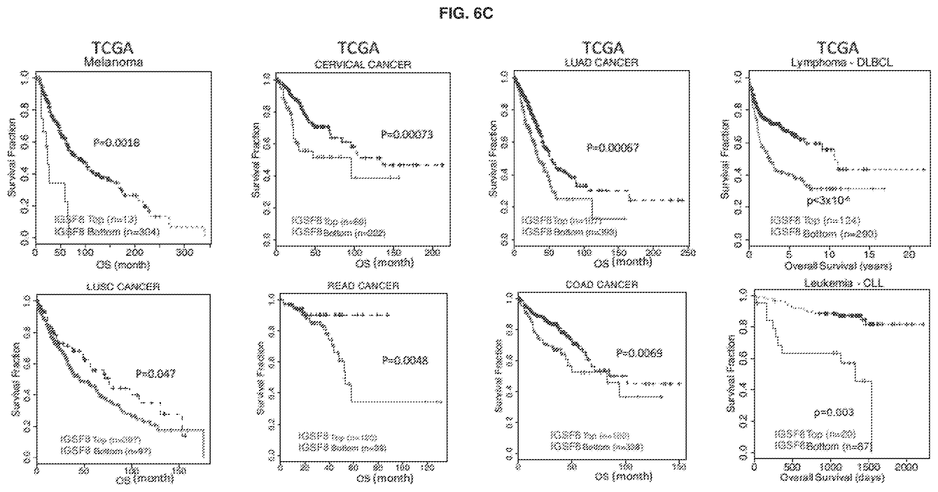

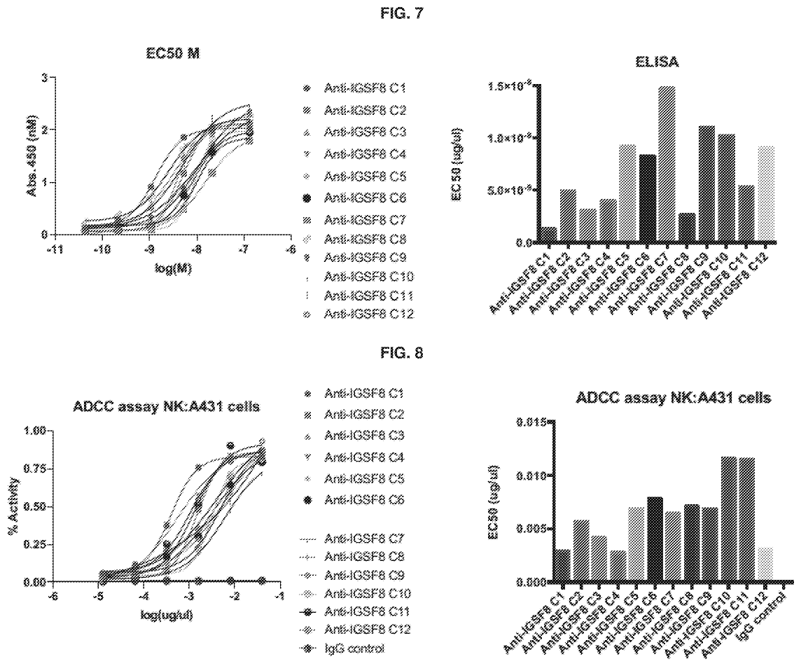

About 50 anti-IGSF8 monoclonal antibodies were produced, twelve of which, anti-IGSF8 C1 to C12, were tested in affinity binding assays using ELISA, all exhibited high affinity for the extracellular domain (ED) of IGSF8. See FIG. 7. The antibodies showing the strongest binding affinity have EC50 values of about mid- to low-nM range. See C1-C₄, C8, and C11.

The sequences of these representative antibodies, including the light chain (LC) and heavy chain (HC) variable regions, the CDR regions, the framework regions (FR), and constant regions, are listed in the table below (H=heavy chain; L=light chain; CDR-H1 to -H3: the three heavy chain CDR sequences; CDR-L1 to -L3: the three light chain CDR sequences; FR: framework region).

| | |
|---|---|
| | Antibody C1 (from top to bottom, SEQ ID NOs: 1-16) |
| CDR-H1 | RYRMS |
| CDR-H2 | RISRSGGATAYADSVKG |
| CDR-H3 | DATGRHYNGMDV |
| CDR-L1 | RASQTITRHLN |
| CDR-L2 | GTSALQT |
| CDR-L3 | QQSHTKPWT |
| HFR1 | QVQLLQSGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGRGTLVTVS |
| LFR1 | EIALTQSPSSLSASVGDRVTITC |
| LFR2 | WFQQKPGKAPNLLIH |
| LFR3 | GVPPRFSGGGSGTDFTLTINSLQPEDFGTYYC |
| LFR4 | FGPGTKVEIKRTV |
| HCVR | QVQLLQSGGGLVQPGGSLRLSCAASGFTFSRYRMSWVRQAPGKGLEWVSRI |
| | SRSGGATAYADSVKGRFTISRDNSKNTLYLQMNSLRAEDTAVYYCARDAT |
| | GRHYNGMDVWGRGTLVTVSS |
| LCVR | EIALTQSPSSLSASVGDRVTITCRASQTITRHLNWFQQKPGKAPNLLIHGTSA |
| | LQTGVPPRFSGGGSGTDFTLTINSLQPEDFGTYYCQQSHTKPWTFGPGTKVEI |
| | KRTV |
| | Antibody C2 (from top to bottom, SEQ ID NOs: 17-32) |
| CDR-H1 | SYPMN |
| CDR-H2 | RISRSGGRTSYADSVKG |
| CDR-H3 | DATRRHYNGMDV |
| CDR-L1 | RASRSVGKYLA |
| CDR-L2 | YASLRAG |
| CDR-L3 | QQYGSSPRT |
| HFR1 | EVQLLQSGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGKGTTVTVS |
| LFR1 | DVVMTQSPATLSLSPGERASLSC |
| LFR2 | WYQQKPGQAPRLLFY |
| LFR3 | DIPSRFTASGSGTDFTLTISRLEPEDFAVYYC |
| LFR4 | FGQGTKLEMKRTV |
| HCVR | EVQLLQSGGGLVQPGGSLRLSCAASGFTFSSYPMNWVRQAPGKGLEWVSRI |
| | SRSGGRTSYADSVKGRFTISRDNSKNTLYLQMNSLRAEDTAVYYCARDATR |
| | RHYNGMDVWGKGTTVTVSS |
| LCVR | DVVMTQSPATLSLSPGERASLSCRASRSVGKYLAWYQQKPGQAPRLLFYYA |
| | SLRAGDIPSRFTASGSGTDFTLTISRLEPEDFAVYYCQQYGSSPRTFGQGTKL |
| | EMKRTV |
| | Antibody C3 (from top to bottom, SEQ ID NOs: 33-48) |
| CDR-H1 | HYPMR |
| CDR-H2 | SIRRSGGRTKYADSVKG |
| CDR-H3 | DATGRHYNGMDV |
| CDR-L1 | RTSQVIGTSLN |
| CDR-L2 | SASNLQS |
| CDR-L3 | QQSSRVPHT |
| HFR1 | QVQLVESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGKGTTVTVS |
| LFR1 | DVVMTQSPSSLSASVGDRVTITC |
| LFR2 | WYQQKPGRAPRLLIY |
| LFR3 | GVPSRFSGSGHGTQFTLTISSLQPEDFATYSC |
| LFR4 | FGQGTKLEMRRTV |
| HCVR | QVQLVESGGGLVQPGGSLRLSCAASGFTFSHYPMRWVRQAPGKGLEWVSSI |
| | RRSGGRTKYADSVKGRFTISRDNSKNTLYLQMNSLRAEDTAVYYCARDAT |
| | GRHYNGMDVWGKGTTVTVSS |
| LCVR | DVVMTQSPSSLSASVGDRVTITCRTSQVIGTSLNWYQQKPGRAPRLLIYSAS |
| | NLQSGVPSRFSGSGHGTQFTLTISSLQPEDFATYSCQQSSRVPHTFGQGTKLE |
| | MRRTV |
| | Antibody C4 (from top to bottom, SEQ ID NOs: 49-64) |
| CDR-H1 | RYRMG |
| CDR-H2 | SIARSGGRTYYADSVKG |
| CDR-H3 | GVRYCSSPSCSRGPRYAMDV |
| CDR-L1 | RASQGISSWLA |
| CDR-L2 | AASSLQS |
| CDR-L3 | QQANSFPIT |
| HFR1 | QVQLLQSGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGKGTTVTVS |
| LFR1 | EIVMTQSPSSVSASVGDRVTITC |
| LFR2 | WYQQKPGKAPKLLIY |
| LFR3 | GVPSRFSGSGSGTDFTLTISSLQPEDFATYYC |
| LFR4 | FGQGTRLEIKRTV |
| HCVR | QVQLLQSGGGLVQPGGSLRLSCAASGFTFSRYRMGWVRQAPGKGLEWVSS |
| | IARSGGRTYYADSVKGRFTISRDNSKNTLYLQMNSLRAEDTAVYYCARGVR |
| | YCSSPSCSRGPRYAMDVWGKGTTVTVSS |
| LCVR | EIVMTQSPSSVSASVGDRVTITCRASQGISSWLAWYQQKPGKAPKLLIYAAS |
| | SLQSGVPSRFSGSGSGTDFTLTISSLQPEDFATYYCQQANSFPITFGQGTRL |
| | EIKRTVAAPSVFIFPPSDEQLKSGTASVVCLLNNFYPREAKVQWKVDNALQS |
| | GNSQESVTEQDSKDSTYSLSSTLTLSKQTTRNTKSTPAKSPIRA |
| | Antibody C5 (from top to bottom, SEQ ID NOs: 65-80) |
| CDR-H1 | RYRMA |
| CDR-H2 | NITRSGGVTRYADSVKG |
| CDR-H3 | DPNRVTAISSHYGMDV |
| CDR-L1 | RASQSISRWLA |
| CDR-L2 | DASNRAT |
| CDR-L3 | QQRSNWPPMYT |
| HFR1 | EVQLVQSGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGKGTTVTVS |
| LFR1 | EIVLTQSPSTLSASVGDRVTISC |
| LFR2 | WYQQKPGQAPRLLIY |
| LFR3 | GVPARFSVSGSETDSTLTISSLEPEDFAMYYC |
| LFR4 | FGQGTKLEIKRTV |
| HCVR | EVQLVQSGGGLVQPGGSLRLSCAASGFTFSRYRMAWVRQAPGKGLEWVSN |
| | ITRSGGVTRYADSVKGRFTISRDNSKNTLYLQMNSLRAEDTAVYYCARDPN |
| | RVTAISSHYGMDVWGKGTTVTVSS |
| LCVR | EIVLTQSPSTLSASVGDRVTISCRASQSISRWLAWYQQKPGQAPRLLIYDASN |
| | RATGVPARFSVSGSETDSTLTISSLEPEDFAMYYCQQRSNWPPMYTFGQGTK |
| | LEIKRTV |
| | Antibody C6 (from top to bottom, SEQ ID NOs: 81-96) |
| CDR-H1 | PYRMH |
| CDR-H2 | RINPSGGRTWYADSVKG |
| CDR-H3 | DATGRHYNGMDV |
| CDR-L1 | RASQSINKWLA |
| CDR-L2 | KASTLES |
| CDR-L3 | QQSHSAPWT |
| HFR1 | EVQLVESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGQGTMVTVSS |
| LFR1 | DIQMTQSPSTLSASVGDRVTITC |
| LFR2 | WYQQKPGKAPKLLIY |
| LFR3 | GVPSRFSGSGSGTDFTLTINSLQPEDFATYYC |
| LFR4 | FGQGTKVEIERTV |
| HCVR | EVQLVESGGGLVQPGGSLRLSCAASGFTFSPYRMHWVRQAPGKGLEWVSRI |
| | NPSGGRTWYADSVKGRFTISRDNSKNTLYLQMNSLRAEDTAVYYCARDAT |
| | GRHYNGMDVWGQGTMVTVSS |
| LCVR | DIQMTQSPSTLSASVGDRVTITCRASQSINKWLAWYQQKPGKAPKLLIYKAS |
| | TLESGVPSRFSGSGSGTDFTLTINSLQPEDFATYYCQQSHSAPWTFGQGTKV |
| | EIERTV |
| | Antibody C7 (from top to bottom, SEQ ID NOs: 97-112) |
| CDR-H1 | SYPMN |
| CDR-H2 | RISRSGGRTSYADSVKG |
| CDR-H3 | DATRRHYNGMDV |
| CDR-L1 | RASRSVGKYLA |
| CDR-L2 | YASLRAG |
| CDR-L3 | QQYGSSPRT |
| HFR1 | EVQLVQSGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGKGTTVTVS |
| LFR1 | ETTLTQSPATLSLSPGERASLSC |
| LFR2 | WYQQKPGQAPRLLFY |
| LFR3 | DIPSRFTASGSGTDFTLTISRLEPEDFAVYYC |
| LFR4 | FGQGTKLEMKRTV |
| HCVR | EVQLEESGGGLVQPGGSLRLSCAASGFTFSSYPMNWVRQAPGKGLEWVSRI |
| | SRSGGRTSYADSVKGRFTISRDNSKNTLYLQMNSLRAEDTAVYYCARDATR |
| | RHYNGMDVWGKGTTVTVSS |
| LCVR | ETTLTQSPATLSLSPGERASLSCRASRSVGKYLAWYQQKPGQAPRLLFYYAS |
| | LRAGDIPSRFTASGSGTDFTLTISRLEPEDFAVYYCQQYGSSPRTFGQGTKLE |
| | MKRTV |
| | Antibody C8 (from top to bottom, SEQ ID NOs: 113-128) |
| CDR-H1 | SYAMS |
| CDR-H2 | AISGSGGSTYYADSVKG |
| CDR-H3 | PYNSAWESYYYGMDV |
| CDR-L1 | RASQGISSRLA |
| CDR-L2 | AASSLQS |
| CDR-L3 | QQRHSYPIT |
| HFR1 | EVQLVQSGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGKGTTVTVS |
| LFR1 | DIQMTQSPSSVSASVGDRVTITC |
| LFR2 | WYQQKPGKAPKLLIY |
| LFR3 | GVPSRFSGSGSGTDFTLTISSLQPEDFATYYC |
| LFR4 | FGQGTRLEIKRTV |
| HCVR | EVQLQESGGGLVQPGGSLRLSCAASGFTFSSYAMSWVRQAPGKGLEWVSAI |
| | SGSGGSTYYADSVKGRFTISRDNSKNTLYLQMNSLRAEDTAVYYCARPYNS |
| | AWESYYYGMDVWGKGTTVTVSS |
| LCVR | DIQMTQSPSSVSASVGDRVTITCRASQGISSRLAWYQQKPGKAPKLLIYAAS |
| | SLQSGVPSRFSGSGSGTDFTLTISSLQPEDFATYYCQQRHSYPITFGQGTRLEI |
| | KRTV |
| | Antibody C9 (from top to bottom, SEQ ID NOs: 129-144) |
| CDR-H1 | RYDMS |
| CDR-H2 | RIRYSGGRTGYADSVKG |
| CDR-H3 | GVRYCSSPSCSRGPRYAMDV |
| CDR-L1 | RASQSVRGYLA |
| CDR-L2 | DTFKRAT |
| CDR-L3 | QQYFASPWT |
| HFR1 | EVQLVQSGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGKGTTVTVS |
| LFR1 | DVVMTQSPATLSLSPGEGATLSC |
| LFR2 | WYQQKPGQAPRLLIY |
| LFR3 | GIPARFSGSGSGADFTLTISSLEPEDSAVYYC |
| LFR4 | FGQGTKVEIKRTV |
| HCVR | EVQLVESGGGLVQPGGSLRLSCAASGFTFSRYDMSWVRQAPGKGLEWVSRI |
| | RYSGGRTGYADSVKGRFTISRDNSKNTLYLQMNSLRAEDTAVYYCARGVR |
| | YCSSPSCSRGPRYAMDVWGKGTTVTVSS |
| LCVR | DVVMTQSPATLSLSPGEGATLSCRASQSVRGYLAWYQQKPGQAPRLLIYDT |
| | FKRATGIPARFSGSGSGADFTLTISSLEPEDSAVYYCQQYFASPWTFGQGT |
| | KVEIKRTV |
| | Antibody C10 (from top to bottom, SEQ ID NOs: 145-160) |
| CDR-H1 | RYRMY |
| CDR-H2 | TISRSGGRTVYADSVKG |
| CDR-H3 | DATGRHYNGMDV |
| CDR-L1 | RASQSVSSNVA |
| CDR-L2 | GSGTRAT |
| CDR-L3 | QQYNDWPS |
| HFR1 | EVQLLESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGQGTLVTV |
| LFR1 | ETTLTQSPATLSVSPGERATLSC |
| LFR2 | WYQQKPGQAPRLLMF |
| LFR3 | GIPARFSGSGSGTEFTLTISSLQSEDFAAYYC |
| LFR4 | FGQGTRVEIKGTV |
| HCVR | EVQLLESGGGLVQPGGSLRLSCAASGFTFSRYRMYWVRQAPGKGLEWVSTI |
| | SRSGGRTVYADSVKGRFTISRDNSKNTLYLQMNSLRAEDTAVYYCARDAT |
| | GRHYNGMDVWGQGTLVTV |
| LCVR | ETTLTQSPATLSVSPGERATLSCRASQSVSSNVAWYQQKPGQAPRLLMFGS |
| | GTRATGIPARFSGSGSGTEFTLTISSLQSEDFAAYYCQQYNDWPSFGQGTR |
| | VEIKGTV |
| | Antibody C11 (from top to bottom, SEQ ID NOs: 161-176) |
| CDR-H1 | RYRMY |
| CDR-H2 | SISSSGGRTKYADSVKG |
| CDR-H3 | GVRYCSSPSCSRGPRYAMDV |
| CDR-L1 | RASYVIRNDLS |
| CDR-L2 | GTSSLHN |
| CDR-L3 | LQDDKYPLT |
| HFR1 | EVQLVQSGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGKGTTVTVS |
| LFR1 | DIQMTQSPSSLSASVGDRVTITC |
| LFR2 | WYQQKPGKAPKLLIY |
| LFR3 | GVPSRFSGSGYGTYFTLTISSLQPEDFGTYYC |
| LFR4 | FGGGTKVEIKRTV |
| HCVR | EVQLVESGGGLVQPGGSLRLSCAASGFTFSRYRMYWVRQAPGKGLEWVSSI |
| | SSSGGRTKYADSVKGRFTISRDNSKNTLYLQMNSLRAEDTAVYYCARGVRY |
| | CSSPSCSRGPRYAMDVWGKGTTVTVSS |
| LCVR | DIQMTQSPSSLSASVGDRVTITCRASYVIRNDLSWYQQKPGKAPKLLIYGTSS |
| | LHNGVPSRFSGSGYGTYFTLTISSLQPEDFGTYYCLQDDKYPLTFGGGTKVEI |
| | KRTV |
| | Antibody C12 (from top to bottom, SEQ ID NOs: 177-192) |
| CDR-H1 | KYKMS |
| CDR-H2 | TIAPSGGGTRYADSVKG |
| CDR-H3 | GGHFSNP |
| CDR-L1 | RSSQSLVHTDGDTYLN |
| CDR-L2 | KVSKRDS |
| CDR-L3 | MQGIKRPYT |
| HFR1 | EVQLVESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGQGTLVTV |
| LFR1 | DVVMTQSPLSLPVTLGQPASISC |
| LFR2 | WYQQRPGQSPRRLIY |
| LFR3 | GVPDRFSGSGSGTDFTLKISRVEAEDVGVYYC |
| LFR4 | LGQGTKLEIKRTV |
| HCVR | EVQLVESGGGLVQPGGSLRLSCAASGFTFSKYKMSWVRQAPGKGLEWVSTI |
| | APSGGGTRYADSVKGRFTISRDNSKNTLYLQMNSLRAEDTAVYYCARGGH |
| | FSNPWGQGTLVTVSS |
| LCVR | DVVMTQSPLSLPVTLGQPASISCRSSQSLVHTDGDTYLNWYQQRPGQSPRRL |
| | IYKVSKRDSGVPDRFSGSGSGTDFTLKISRVEAEDVGVYYCMQGIKRPYTLG |
| | QGTKLEIKRTVAAPSVFIFPPSDEQLKSGTASVVCLLNNFYPREAKVQWKVD |
| | NALQSGNSQESVTEQDSKDSTYSLSSTLTLSKADYEKHKLYACEVTHQGLSS |
| | PVTKSFNRGEC |
| | Antibody C13 (from top to bottom, SEQ ID NOs: 193-208) |
| CDR-H1 | PYRMH |
| CDR-H2 | SINRSGGRTNYADSVKG |
| CDR-H3 | GRGIGTFRN |
| CDR-L1 | RASQSVSTYLA |
| CDR-L2 | DASNRAT |
| CDR-L3 | QQRNNWPPT |
| HFR1 | EVQLVESGGGLVQPGGSLRLSCAASGFTFS |
| HFR2 | WVRQAPGKGLEWVS |
| HFR3 | RFTISRDNSKNTLYLQMNSLRAEDTAVYYCAT |
| HFR4 | WGQGTLVTVSS |
| LFR1 | DIALTQSPATLSLSPGERATLSC |
| LFR2 | WYQQKPGQAPRLLIS |
| LFR3 | GIPARFSGSGSGTDFTLTISSLEPEDFAVYYC |
| LFR4 | FGQGTKVEIKRTV |
| HCVR | EVQLVESGGGLVQPGGSLRLSCAASGFTFSPYRMHWVRQAPGKGLEWVSSI |
| | NRSGGRTNYADSVKGRFTISRDNSKNTLYLQMNSLRAEDTAVYYCATGRGI |
| | GTFRNWGQGTLVTVSS |
| LCVR | DIALTQSPATLSLSPGERATLSCRASQSVSTYLAWYQQKPGQAPRLLISDASN |
| | RATGIPARFSGSGSGTDFTLTISSLEPEDFAVYYCQQRNNWPPTFGQGTKVEI |
| | KRTVAAPSVFIFPPSDEQLKSGTASVVCLLNNFYPREAKVQWKVDNALQSG |
| | NSQESVTEQDSKDSTYSLSSTLTLSKADYEKHKLYACEVTHQGLSSPVTKSF |
| | NRGEC |
| | Antibody C14 (from top to bottom, SEQ ID NOs: 209-224) |
| CDR-H1 | SYAMS |
| CDR-H2 | AISGSGGSTYYADSVKG |
| CDR-H3 | DTIPGYMDV |
| CDR-L1 | RASQSISNYLS |
| CDR-L2 | AASSLQS |
| CDR-L3 | QQSYSSPYT |
| HFR1 | EVQLLESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGQGTLVTV |
| LFR1 | DIMLTQSPSSLSGSVGDSVTFTC |
| LFR2 | WYQQKSGKAPQLLIY |
| LFR3 | GVPSRFSGSGSGTDFTLTISSLQPEDFATYYC |
| LFR4 | FGQGTKLEIKRTV |
| HCVR | EVQLVESGGGLVQPGGSLRLSCAASGFTFSSYAMSWVRQAPGKGLEWVSAI |
| | SGSGGSTYYADSVKGRFTISRDNSKNTLYLQMNSLRAEDTAVYYCARDTIP |
| | GYMDVWGKGTTVTVSS |
| LCVR | DIMLTQSPSSLSGSVGDSVTFTCRASQSISNYLSWYQQKSGKAPQLLIYAASS |
| | LQSGVPSRFSGSGSGTDFTLTISSLQPEDFATYYCQQSYSSPYTFGQGTKLEIK |
| | RTVAAPSVFIFPPSDEQLKSGTASVVCLLNNFYPREAKVQWKVDNALQSGN |
| | SQESVTEQDSKDSTYSLSSTLTLSKADYEKHKLYACEVTHQGLSSPVTKSFN |
| | RGEC |
| | Antibody C15 (from top to bottom, SEQ ID NOs: 225-240) |
| CDR-H1 | RYRMA |
| CDR-H2 | AIARSGGRTWYADSVKG |
| CDR-H3 | GGGAKWLYNWFDS |
| CDR-L1 | RASQSVSNTYLA |
| CDR-L2 | GASIRAP |
| CDR-L3 | QQYARSRIA |
| HFR1 | EVQLVESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGQGTLVTV |
| LFR1 | EIVLTQSPGTLSLSAGERATLSC |
| LFR2 | WYQQKPGQAPRLLIY |
| LFR3 | GIPDRFSGSGSGTDFTLTVNRLEPEDSAVYYC |
| LFR4 | FGQGTRLEIRRTV |
| HCVR | LRGGISRARLVNRQIAWRRHPRCFDLHRRHRDRSSLRTRPQTTRQTCKRRH |
| | AQLSTALLPGPPDWGEGPGAAGAVGVLLTGVRAEVQLVESGGGLVQPGGS |
| | LRLSCAASGFTFSRYRMAWVRQAPGKGLEWVSAIARSGGRTWYADSVKGR |
| | FTISRDNSKNTLYLQMNSLRAEDTAVYYCARGGGAKWLYNWFDS |
| LCVR | EIVLTQSPGTLSLSAGERATLSCRASQSVSNTYLAWYQQKPGQAPRLLIYGA |
| | SIRAPGIPDRFSGSGSGTDFTLTVNRLEPEDSAVYYCQQYARSRIAFGQGTRL |
| | EIRRTV |
| | Antibody C16 (from top to bottom, SEQ ID NOs: 241-256) |
| CDR-H1 | HYWMG |
| CDR-H2 | GIGASGGWTGYADSVKG |
| CDR-H3 | TSGAYFDY |
| CDR-L1 | RASQSVSSDYLA |
| CDR-L2 | GASSRAT |
| CDR-L3 | QQYGSTPLT |
| HFR1 | EVQLLESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGQGTLVTV |
| LFR1 | EIVLTQSPGTLSLSPGQRATLSC |
| LFR2 | WYQQKPGQAPRLLMY |
| LFR3 | GIPDRFSGSGSGTDFTLTISRLEPEDFAVYYC |
| LFR4 | FGGGTTVEIRRTV |
| HCVR | EVQLVESGGGLVQPGGSLRLSCAASGFTFSHYWMGWVRQAPGKGLEWVS |
| | GIGASGGWTGYADSVKGRFTISRDNSKNTLYLQMNSLRAEDTAVYYCARTS |
| | GAYFDYWGQGTLVTVSS |
| LCVR | EIVLTQSPGTLSLSPGQRATLSCRASQSVSSDYLAWYQQKPGQAPRLLMYG |
| | ASSRATGIPDRFSGSGSGTDFTLTISRLEPEDFAVYYCQQYGSTPLTFGGGTT |
| | VEIRRTV |
| | Antibody C17 (from top to bottom, SEQ ID NOs: 257-272) |
| CDR-H1 | NYPMT |
| CDR-H2 | TIRGSGGDTWYADSVKG |
| CDR-H3 | WVGRDA |
| CDR-L1 | RSSQSLVYSDGNTYLN |
| CDR-L2 | KVSNRDS |
| CDR-L3 | MQGTHWPPT |
| HFR1 | EVQLLESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGQGTLVTV |
| LFR1 | DIVLTQSPLSLPVTLGQPASISC |
| LFR2 | WFRQRPGQSPRRLIY |
| LFR3 | GVPDRFSGSGSGTDFTLRISRVEAEDVGVYYC |
| LFR4 | FGQGTKLEIKRTV |
| HCVR | EVQLVESGGGLVQPGGSLRLSCAASGFTFSNYPMTWVRQAPGKGLEWVSTI |
| | RGSGGDTWYADSVKGRFTISRDNSKNTLYLQMNSLRAEDTAVYYCAKWV |
| | GRDAWGQGTLVTVSS |
| LCVR | DIVLTQSPLSLPVTLGQPASISCRSSQSLVYSDGNTYLNWFRQRPGQSPRRLI |
| | YKVSNRDSGVPDRFSGSGSGTDFTLRISRVEAEDVGVYYCMQGTHWPPTFG |
| | QGTKLEIKRTV |
| | Antibody C18 (from top to bottom, SEQ ID NOs: 273-288) |
| CDR-H1 | SYPMN |
| CDR-H2 | RISRSGGRTSYADSVKG |
| CDR-H3 | DATRRHYNGMDV |
| CDR-L1 | RASRSVGKYLA |
| CDR-L2 | YASLRAG |
| CDR-L3 | QQYGSSPRT |
| HFR1 | EVQLVESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGKGTTVTVSS |
| LFR1 | DIVLTQSPATLSLSPGERASLSC |
| LFR2 | WYQQKPGQAPRLLFY |
| LFR3 | DIPSRFTASGSGTDFTLTISRLEPEDFAVYYC |
| LFR4 | FGQGTKLEMKRTV |
| HCVR | EVQLVESGGGLVQPGGSLRLSCAASGFTFSSYPMNWVRQAPGKGLEWVSRI |
| | SRSGGRTSYADSVKGRFTISRDNSKNTLYLQMNSLRAEDTAVYYCARDATR |
| | RHYNGMDVWGKGTTVTVSS |
| LCVR | DIVLTQSPATLSLSPGERASLSCRASRSVGKYLAWYQQKPGQAPRLLFYYAS |
| | LRAGDIPSRFTASGSGTDFTLTISRLEPEDFAVYYCQQYGSSPRTFGQGTKLE |
| | MKRTV |
| | Antibody C19 (from top to bottom, SEQ ID NOs: 289-304) |
| CDR-H1 | RYRMH |
| CDR-H2 | SIASSGGRTRYADSVKG |
| CDR-H3 | GGLPYRGHYGMDV |
| CDR-L1 | RASQSISSYLN |
| CDR-L2 | VASSLQS |
| CDR-L3 | QQARSIPWT |
| HFR1 | EVQLVESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGQGTLVTVSS |
| LFR1 | EIMLTQSPSSLSASVGDRVTITC |
| LFR2 | WYQQKPGKAPKLLIS |
| LFR3 | GVPSRFSGSRSGTDFTLTISSLQPEDFATYYC |
| LFR4 | FGQGTNVEIKRTV |
| HCVR | EVQLVESGGGLVQPGGSLRLSCAASGFTFSRYRMHWVRQAPGKGLEWVSSI |
| | ASSGGRTRYADSVKGRFTISRDNSKNTLYLQMNSLRAEDTAVYYCARGGLP |
| | YRGHYGMDVWGQGTLVTVSS |
| LCVR | EIMLTQSPSSLSASVGDRVTITCRASQSISSYLNWYQQKPGKAPKLLISVASS |
| | LQSGVPSRFSGSRSGTDFTLTISSLQPEDFATYYCQQARSIPWTFGQGTNVEI |
| | KRTV |
| | Antibody C20 (from top to bottom, SEQ ID NOs: 305-320) |
| CDR-H1 | SYAMS |
| CDR-H2 | AISGSGGSTYYADSVKG |
| CDR-H3 | GGLPYRGHYGMDV |
| CDR-L1 | RSSQSLLHSNGYNYVD |
| CDR-L2 | LGSNRAS |
| CDR-L3 | MQALKIPRT |
| HFR1 | EVQLVESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGQGTLVTVSS |
| LFR1 | DIVLTQSPLSLPVTPGEPASISC |
| LFR2 | WYLQKPGQSPQLLIY |
| LFR3 | GVPDRFSGSGSGTDFTLKISRVEAEDVGVYYC |
| LFR4 | FGQGTKVEIKRTV |
| HCVR | EVQLVESGGGLVQPGGSLRLSCAASGFTFSSYAMSWVRQAPGKGLEWVSAI |
| | SGSGGSTYYADSVKGRFTISRDNSKNTLYLQMNSLRAEDTAVYYCARGGLP |
| | YRGHYGMDVWGQGTLVTVSS |
| LCVR | DIVLTQSPLSLPVTPGEPASISCRSSQSLLHSNGYNYVDWYLQKPGQSPQLLI |
| | YLGSNRASGVPDRFSGSGSGTDFTLKISRVEAEDVGVYYCMQALKIPRTFGQ |
| | GTKVEIKRTV |
| | Antibody C21 (from top to bottom, SEQ ID NOs: 321-336) |
| CDR-H1 | PYYMV |
| CDR-H2 | SINRSGGRTAYADSVKG |
| CDR-H3 | AIAAGRYGMDV |
| CDR-L1 | RASQSVSSYLA |
| CDR-L2 | DASNRAT |
| CDR-L3 | QQRTNWPPLT |
| HFR1 | EVQLVESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGQGTLVTVSS |
| LFR1 | DIEMTQSPATLSLSPGERATLSC |
| LFR2 | WYQQKPGQPPRLLIY |
| LFR3 | GIPARFSGSGSGTDFTLTISSLEPEDFAVYYC |
| LFR4 | FGGGTKVEIKRTV |
| HCVR | EVQLVESGGGLVQPGGSLRLSCAASGFTFSPYYMVWVRQAPGKGLEWVSSI |
| | NRSGGRTAYADSVKGRFTISRDNSKNTLYLQMNSLRAEDTAVYYCARAIAA |
| | GRYGMDVWGKGTTVTVSS |
| LCVR | DIEMTQSPATLSLSPGERATLSCRASQSVSSYLAWYQQKPGQPPRLLIYDAS |
| | NRATGIPARFSGSGSGTDFTLTISSLEPEDFAVYYCQQRTNWPPLTFGGGTKV |
| | EIKRTV |
| | Antibody C22 (from top to bottom, SEQ ID NOs: 337-352) |
| CDR-H1 | RYTMR |
| CDR-H2 | GISRSGGRTVYADSVKG |
| CDR-H3 | DPFGVVNHFYYMDV |
| CDR-L1 | RASQSIHTYLN |
| CDR-L2 | GASNLQN |
| CDR-L3 | QQTYRTPTT |
| HFR1 | EVQLVESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGKGTTVTVSS |
| LFR1 | EIMLTQSPPSLSASVGDRVTITC |
| LFR2 | WYQQKPGKAPKLLIY |
| LFR3 | GVPSRFSGTGSGTDFALTISSLQPEDFATYSC |
| LFR4 | FGPGTKVDIKRTV |
| HCVR | EVQLVESGGGLVQPGGSLRLSCAASGFTFSRYTMRWVRQAPGKGLEWVSGI |
| | SRSGGRTVYADSVKGRFTISRDNSKNTLYLQMNSLRAEDTAVYYCARDPFG |
| | VVNHFYYMDVWGKGTTVTVSS |
| LCVR | EIMLTQSPPSLSASVGDRVTITCRASQSIHTYLNWYQQKPGKAPKLLIYGASN |
| | LQNGVPSRFSGTGSGTDFALTISSLQPEDFATYSCQQTYRTPTTFGPGTKVDI |
| | KRTV |
| | Antibody C23 (from top to bottom, SEQ ID NOs: 353-368) |
| CDR-H1 | SYRMS |
| CDR-H2 | GIGRSGGRTRYADSVKG |
| CDR-H3 | AIAAGRYGMDV |
| CDR-L1 | RASQSIRNNYLA |
| CDR-L2 | GASYRAT |
| CDR-L3 | QQRSNWPPT |
| HFR1 | EVQLVESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGKGTTVTVSS |
| LFR1 | DIMLTQSPGTLSLSPGERATLSC |
| LFR2 | WYQQRPGQAPRLLIY |
| LFR3 | GIPDRFSGSGSGTDFTLTISSLEPEDFAVYYC |
| LFR4 | FGGGTKVEIKRTV |
| HCVR | EVQLVESGGGLVQPGGSLRLSCAASGFTFSSYRMSWVRQAPGKGLEWVSGI |
| | GRSGGRTRYADSVKGRFTISRDNSKNTLYLQMNSLRAEDTAVYYCARAIAA |
| | GRYGMDVWGKGTTVTVSS |
| LCVR | DIMLTQSPGTLSLSPGERATLSCRASQSIRNNYLAWYQQRPGQAPRLLIYGA |
| | SYRATGIPDRFSGSGSGTDFTLTISSLEPEDFAVYYCQQRSNWPPTFGGGTKV |
| | EIKRTV |
| | Antibody C24 (from top to bottom, SEQ ID NOs: 369-384) |
| CDR-H1 | RYPMV |
| CDR-H2 | RISRSGGRTQYADSVKG |
| CDR-H3 | DATGRHYNGMDV |
| CDR-L1 | RASQSISSYLN |
| CDR-L2 | GASSLQS |
| CDR-L3 | QQANSFPLT |
| HFR1 | EVQLVESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGQGTLVTVSS |
| LFR1 | EIAMTQSPSSLSASVGDRVTITC |
| LFR2 | WYQQKPGKAPKLLIY |
| LFR3 | GVPSRFSGSGSGTDFTLTISSLQPEDFATYYC |
| LFR4 | FGGGTKVEIKRTV |
| HCVR | EVQLVESGGGLVQPGGSLRLSCAASGFTFSRYPMVWVRQAPGKGLEWVSRI |
| | SRSGGRTQYADSVKGRFTISRDNSKNTLYLQMNSLRAEDTAVYYCARDAT |
| | GRHYNGMDVWGQGTLVTVSS |
| LCVR | EIAMTQSPSSLSASVGDRVTITCRASQSISSYLNWYQQKPGKAPKLLIYGASS |
| | LQSGVPSRFSGSGSGTDFTLTISSLQPEDFATYYCQQANSFPLTFGGGTKVEI |
| | KRTV |
| | Antibody C25 (from top to bottom, SEQ ID NOs: 385-400) |
| CDR-H1 | SYRMS |
| CDR-H2 | GIGRSGGRTRYADSVKG |
| CDR-H3 | AIAAGRYGMDV |
| CDR-L1 | RASQSIRNNYLA |
| CDR-L2 | GASYRAT |
| CDR-L3 | QQRSNWPPT |
| HFR1 | EVQLVESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGKGTTVTVSS |
| LFR1 | EIELTQSPGTLSLSPGERATLSC |
| LFR2 | WYQQRPGQAPRLLIY |
| LFR3 | GIPDRFSGSGSGTDFTLTISSLEPEDFAVYYC |
| LFR4 | FGGGTKVEIKRTV |
| HCVR | EVQLVESGGGLVQPGGSLRLSCAASGFTFSSYRMSWVRQAPGKGLEWVSGI |
| | GRSGGRTRYADSVKGRFTISRDNSKNTLYLQMNSLRAEDTAVYYCARAIAA |
| | GRYGMDVWGKGTTVTVSS |
| LCVR | EIELTQSPGTLSLSPGERATLSCRASQSIRNNYLAWYQQRPGQAPRLLIYGAS |
| | YRATGIPDRFSGSGSGTDFTLTISSLEPEDFAVYYCQQRSNWPPTFGGGTKVE |
| | IKRTV |
| | Antibody C26 (from top to bottom, SEQ ID NOs: 401-416) |
| CDR-H1 | RYRMA |
| CDR-H2 | GISYSGGETLYADSVKG |
| CDR-H3 | DVRWLQGLDN |
| CDR-L1 | RSSQSLLHTNGNNYLD |
| CDR-L2 | LGSNRAS |
| CDR-L3 | MQTLQTPLT |
| HFR1 | EVQLVESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGQGTLVTVSS |
| LFR1 | EIKLTQSPLSLPVTPGEPASISC |
| LFR2 | WYLQKPGQSPQLLIY |
| LFR3 | GVPDRFSGSGSGTDFTLKISRVEAEDVGVYYC |
| LFR4 | FGGGTKVEIKRTV |
| HCVR | EVQLVESGGGLVQPGGSLRLSCAASGFTFSRYRMAWVRQAPGKGLEWVSG |
| | ISYSGGETLYADSVKGRFTISRDNSKNTLYLQMNSLRAEDTAVYYCARDVR |
| | WLQGLDNWGQGTLVTVSS |
| LCVR | EIKLTQSPLSLPVTPGEPASISCRSSQSLLHTNGNNYLDWYLQKPGQSPQLLI |
| | YLGSNRASGVPDRFSGSGSGTDFTLKISRVEAEDVGVYYCMQTLQTPLTFG |
| | GGTKVEIKRTV |
| | Antibody C27 (from top to bottom, SEQ ID NOs: 417-432) |
| CDR-H1 | SYAMS |
| CDR-H2 | AISGSGGSTYYADSVKG |
| CDR-H3 | EGRPGYMDV |
| CDR-L1 | RTSLSIATYLH |
| CDR-L2 | HASSLQT |
| CDR-L3 | QQSYSSPYT |
| HFR1 | EVQLVESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGQGTLVTVSS |
| LFR1 | EIVLTQSPSLLSASVGDRVTITC |
| LFR2 | WYQQKPGRAPKLLIY |
| LFR3 | GVPSRFSGSGSGTDFTLTISSLLPEDFATYFC |
| LFR4 | FGRGTKLEIKRTV |
| HCVR | EVQLVESGGGLVQPGGSLRLSCAASGFTFSSYAMSWVRQAPGKGLEWVSAI |
| | SGSGGSTYYADSVKGRFTISRDNSKNTLYLQMNSLRAEDTAVYYCAREGRP |
| | GYMDVWGQGTLVTVSS |
| LCVR | EIVLTQSPSLLSASVGDRVTITCRTSLSIATYLHWYQQKPGRAPKLLIYHASS |
| | LQTGVPSRFSGSGSGTDFTLTISSLLPEDFATYFCQQSYSSPYTFGRGTKLEIK |
| | RTV |
| | Antibody C28 (from top to bottom, SEQ ID NOs: 433-448) |
| CDR-H1 | VYGMI |
| CDR-H2 | GIPPSGGVTLYADSVKG |
| CDR-H3 | GNYGMDV |
| CDR-L1 | RASQSVSSYLA |
| CDR-L2 | DASNRAT |
| CDR-L3 | QQRSNWPPT |
| HFR1 | EVQLVESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGQGTLVTVSS |
| LFR1 | EIALTQSPATLSLSPGERATLSC |
| LFR2 | WYQQKPGQAPRLLIY |
| LFR3 | GIPARFSGSGSGTDFTLTISSLEPEDFAVYYC |
| LFR4 | FGGGTKVEIKRTV |
| HCVR | EVQLVESGGGLVQPGGSLRLSCAASGFTFSVYGMIWVRQAPGKGLEWVSGI |
| | PPSGGVTLYADSVKGRFTISRDNSKNTLYLQMNSLRAEDTAVYYCARGNYG |
| | MDVWGKGTTVTVSS |
| LCVR | EIALTQSPATLSLSPGERATLSCRASQSVSSYLAWYQQKPGQAPRLLIYDASN |
| | RATGIPARFSGSGSGTDFTLTISSLEPEDFAVYYCQQRSNWPPTFGGGTKVEI |
| | KRTV |
| | Antibody C29 (from top to bottom, SEQ ID NOs: 449-464) |
| CDR-H1 | NYPMT |
| CDR-H2 | TIRGSGGDTWYADSVKG |
| CDR-H3 | WVGRDA |
| CDR-L1 | RSSQSLVYSDGNTYLN |
| CDR-L2 | KVSNRDS |
| CDR-L3 | MQGTHWPYT |
| HFR1 | EVQLVESGGGLVQPGGSLRLSCAASGFTFS |
| HFR2 | WVRQAPGKGLEWVS |
| HFR3 | RFTISRDNSKNTLYLQMNSLRAEDTAVYYCAR |
| HFR4 | WGQGTLVTVSS |
| LFR1 | DIQLTQSPLSLPVTLGQPASISC |
| LFR2 | WFQQRPGQSPRRLIY |
| LFR3 | GVPDRFSGSVSGPDFTLKISRVEAEDVGVYYC |
| LFR4 | FGQGTKLEIKRTV |
| HCVR | EVQLVESGGGLVQPGGSLRLSCAASGFTFSNYPMTWVRQAPGKGLEWVSTI |
| | RGSGGDTWYADSVKGRFTISRDNSKNTLYLQMNSLRAEDTAVYYCAKWV |
| | GRDAWGQGTLVTVSS |
| LCVR | DIQLTQSPLSLPVTLGQPASISCRSSQSLVYSDGNTYLNWFQQRPGQSPRRLI |
| | YKVSNRDSGVPDRFSGSVSGPDFTLKISRVEAEDVGVYYCMQGTHWPYTFG |
| | QGTKLEIKRTV |

In all the above sequences, HCVR (heavy chain variable region) sequence can be assembled based on the disclosed sequences of HFR1/CDR-H1/HFR2/CDR-H2/HFR3/CDR-H3/HFR4 (N to C terminus), plus the most N-terminal signal peptide sequence of

(SEQ ID NO: 465)
MHSSALLCCLVLLTGVRA.

Likewise, LCVR (light chain variable region) sequence can be assembled based on the disclosed sequences of LFR1/CDR-L1/LFR2/CDR-L2/LFR3/CDR-L3/LFR4 (N to C terminus), plus the most N-terminal signal sequence of MHSSALLCCLVLLTGVRA (SEQ ID NO: 465).

One human light chain constant region sequence is shown below:

(SEQ ID NO: 466)
AAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFN
RGEC

The human IgG1 heavy chain constant region sequences are shown as follows:

(SEQ ID NO: 467)
ASTKGPSVFPLAPSSKSTSGGTAALGCLVKDYFPEPVTVSWNSGALTSGV
HTFPAVLQSSGLYSLSSWTVPSSSLGTQTYICNVNHKPSNTKVDKKVEPK
SCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQ
QGNVFSCSVMHEALHNHYTQKSLSLSPGK

Although for the in vivo assays described in this application, only the human IgG1 anti-IGSF8 antibodies were used, other anti-IGSF8 antibodies with other Ig constant regions (such as IgG2, IgG3, IgG4, IgA, IgE, IgM, IgD constant regions) are also contemplated and within the scope of the invention.

### Example 8 Anti-IGSF8 Antibodies Exhibit Strong ADCC Effects

This experiment demonstrates that anti-IGSF8 antibodies of the invention exhibit strong ADCC effects using NK cells as effector cells and A431 cancer cells as target cells.

Here, ADCC (antibody-dependent cell-mediated cytotoxicity) stands for an immune response in which antibodies, by coating target cells, make them vulnerable to attack by immune cells. Specifically, IGSF8 expressed on A431 cancer cell surface was recognized and bound by an increasing concentration of anti-IGSF8 antibodies. The Fc regions of the anti-IGSF8 antibodies were in turn recognized by CD16 Fc receptors on NK cells. Cross-linking of the CD16 Fc receptors triggers a degranulation into a lytic synapse. As a result, the targeted tumor cells were killed via apoptosis.

A431 cells were seeded in 96-well plates with RPMI medium, and incubated for about 1 hour with varying concentrations of the anti-IGSF8 isotypes. Activated primary NK cells from donors were then added to the A431 cells- and antibody-containing wells at 4,000 cells/well (a target:effector ratio of 1:2.5), and incubated for 4 more hours at 37° C. Cell death was determined by lactate dehydrogenase (LDH) release assays.

A dose-response curve was established for each of the 12 tested antibodies C1-C12, and their EC₅₀ values were determined.

All 12 tested anti-IGSF8 antibodies (C1-C12) showed about 3-12 mM range ADCC EC₅₀ values against the A431 cancer cells.

### Example 9 Anti-IGSF8 Antibodies Stimulate CXCL10 Expression

FIG. 4 above shows that inactivating IGSF8 in Colo205 cancer cells using CRISPR/Cas9-mediated gene editing caused a near 7-10 fold increased expression/secretion of CXCL10 by Colo205 cells. This experiment shows that incubating the Colo205 cancer cells with the anti-IGSF8 antibodies of the invention (10 μg/mL) can similarly lead to CXCL10 expression/secretion, based on ELISA.

Specifically, Colo205 cancer cells were seeded in 96 well plates (4,000 cells per well) and cultured with RPMI medium for 12 hours, before one of the test antibodies was added at 5 μg/mL for 24 hours at 37° C. in a humidified atmosphere of 5% CO₂. The supernatant of the media was then collected for standard ELISA assay to determine the titer/amount of CXCL10 in the medium by using a commercial CXCL10 ELISA kit. Antibodies C1-C₄, C8, and C10 all induced relatively high levels of CXCL10 expression by Colo205 cells.

### Example 10 Anti-IGSF8 Antibodies Showed In Vivo Efficacy

In FIGS. 3A-3B, it was shown that knocking out IGSF8 using CRISPR/Cas9-mediated gene editing led to retarded B16-F10 melanoma growth in vivo in a mouse xenograph model, without affecting in vitro tumor cell growth rate per se.

In this experiment, the effect of representative anti-IGSF8 monoclonal antibodies of the invention on tumor growth in B16 syngeneic mouse model was tested. In particular, one million B16-F10 melanoma cells were injected subcutaneously into wild type (WT) C57BL/6 mice. Mice were then treated with one of four anti-IGSF8 antibodies (C1-C4) at a dose of 2 mg/kg, or a control human IgG1, from day 6, every 3 days, for four doses in total by tail vein injection. Data are presented as mean±s.e.m. (n=8 mice per group).

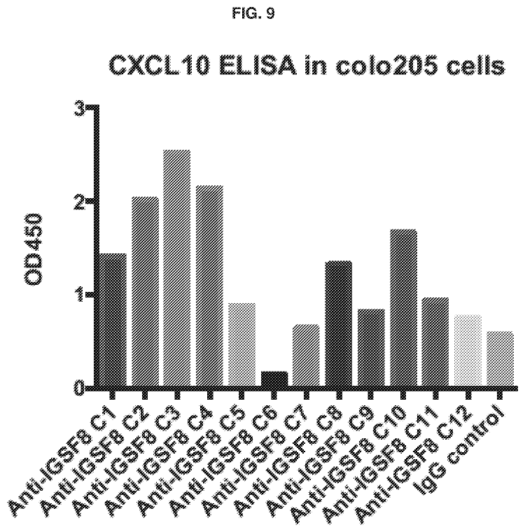

It is apparent that, in wild-type host mice, the subject anti-IGSF8 monoclonal antibodies similarly retarded B16-F10 melanoma tumor growth (volume increase), such that the difference compared to the IgG1 control became statistically significant (p<0.005) after about 18 days for at least C3 and C4. See FIG. 10.

Similar experiments were repeated in nude mice (Foxn1^{nu}), which lack thymus and cannot produce mature T lymphocytes, but have B cells and robust NK cell responses. The effects of the subject anti-IGSF8 antibodies appeared to be similar. At Day 14, the effect of the C2 antibody was statistically significant (p<0.05), so was the effect of C4 (p<0.005).

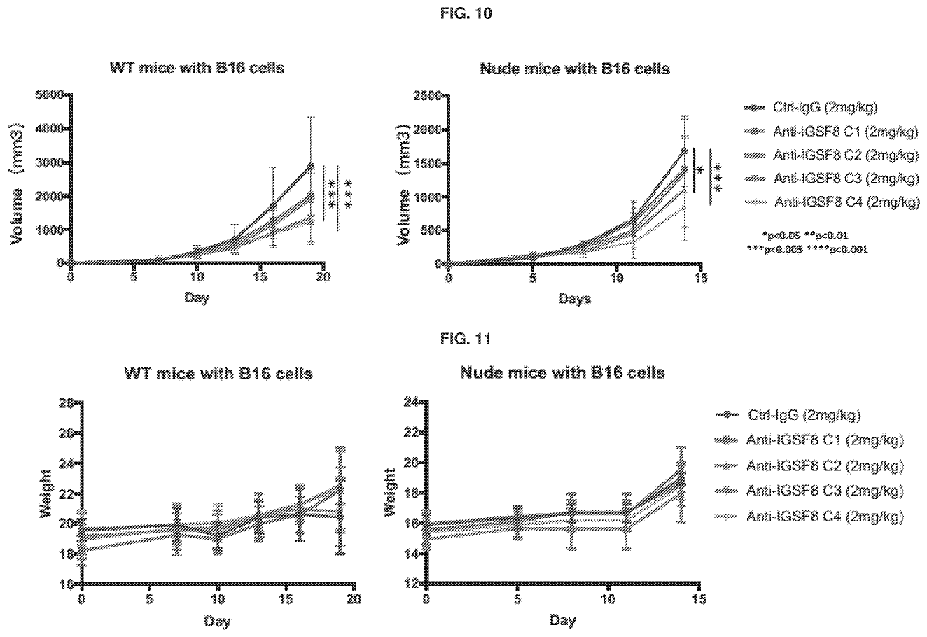

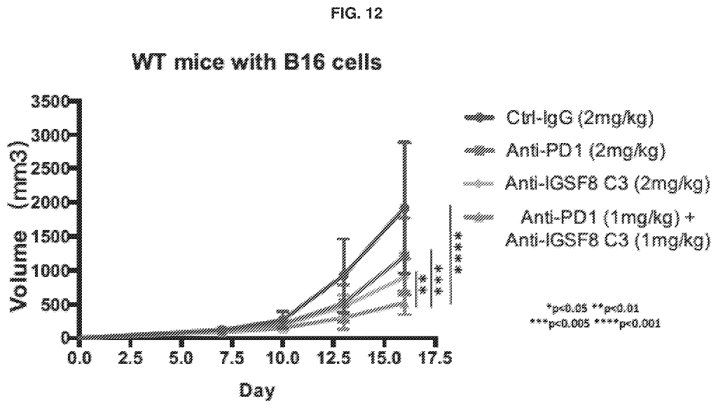

Notably, there did not appear to be any significant weight differences among the different groups of experimental mice (FIG. 11), which result was consistent with the fact that knocking out IGSF8 using CRISPR/Cas9 did not have appreciable effect on tumor cell growth rate per se.

### Example 11 Synergistic Anti-Tumor Effect by Anti-IGSF8 Antibody and Anti-PD-1 Antibody

This experiment demonstrates that the anti-IGSF8 monoclonal antibodies of the invention and anti-PD-1 antibody have synergistic effect in inhibiting B16-F10 melanoma tumor growth in vivo in a syngeneic mouse model.

In particular, one million B16-F10 melanoma cells were injected subcutaneously into wild type (WT) C57BL/6 mice. Mice were then treated, by tail vein injection, with one of four antibodies or antibody combinations: IgG control at a dose of 2 mg/kg, anti-PD-1 antibody at a dose of 2 mg/kg, anti-IGSF8 antibody C3 at a dose of 2 mg/kg, or a combination of anti-PD-1 antibody at half the dose (1 mg/kg) and anti-IGSF8 antibody at half the dose (1 mg/kg). The first doses were administered on Day 6, and subsequent doses were administered every 3 days, for four doses in total. Data are presented as mean±s.e.m. (n=8 mice per group).

It is apparent that the subject anti-IGSF8 antibody and anti-PD-1 antibody exhibited synergistic effect in inhibiting melanoma growth in vivo, in that the combination therapy, administered at a 50% dose (1 mg/kg) for each component of the combination, was statistically significantly better than (1) the anti-IGSF8 antibody C3 alone at twice the dose (2 mg/kg) (p<0.01), (2) the commercial anti-PD-1 antibody (Clone 29F.1A12, BioXcell) alone at twice the dose (2 mg/kg) (p<0.005), and (3) IgG control (p<0.001).

This surprising finding strongly suggests that simultaneously inhibiting the IGSF8 pathway and the PD-1/PD-L1 immune checkpoint can synergistically inhibit tumor growth in vivo.

1. A method of treating cancer in a subject in need thereof, the method comprising administering to the subject a therapeutically effective amount of an IGSF8 (Immuno Globulin Super Family 8) antagonist.

2. The method of claim 1, further comprising administering to the subject an effective amount of a second therapeutic agent selected from the group consisting of: an immune checkpoint inhibitor, a chemotherapeutic agent, an anti-angiogenesis agent, a growth inhibitory agent, an immune-oncology agent, and an anti-neoplastic composition.

3. The method of claim 1 or 2, wherein the IGSF8 antagonist is an anti-IGSF8 antibody, or an antigen-binding portion/fragment thereof.

4. The method of claim 3, wherein the antibody is a chimeric antibody, a humanized antibody, or a human antibody.

5. The method of claim 3 or 4, wherein the antigen-binding portion/fragment is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

6. The method of any one of claims 1 to 5, wherein the cancer is melanoma (including skin cutaneous melanoma), cervical cancer, lung cancer (e.g., non-small cell lung cancer, lung adenocarcinoma, lung squamous cell carcinoma), colorectal cancer, lymphoma (including DLBCL), leukemia (including CLL), BLCA tumor, breast cancer, head-neck squamous cell carcinoma, PRAD, THCA, or UCEC, thyroid cancer, unitary tract cancer, esophagus cancer, liver cancer, or ganglia cancer.

7. The method of any one of claims 1 to 6, wherein the IGSF8 antagonist promotes expression, secretion, or otherwise increases activity of a cytokine or a target gene selected from the group consisting of: CXCL10, CXCL9, TNFα, CD8b, CD8a, Prf1, IFNγ, Gzma, Gzmb, CD274, PDCD1, PDCD1 Ig2, LAG3, Havcr2, Tigit, or CTLA4.

8. The method of any one of claims 1 to 7, wherein expression, secretion, or otherwise increased activity of said cytokine or said target gene occurs within tumor microenvironment.

9. The method of any one of claims 1 to 8, wherein expression, secretion, or otherwise increased activity of said cytokine or said target gene is due to immune cell (e.g., T lymphocytes or NK cells) infiltration into tumor microenvironment.

10. The method of any one of claims 1 to 9, wherein the IGSF8 antagonist is an immunostimulatory molecule.

11. The method of claim 10, wherein the IGSF8 antagonist stimulates T cell or NK cell activation and/or infiltration into tumor microenvironment.

12. The method of any one of claims 1 to 11, wherein the immune checkpoint inhibitor is an antibody or antigen-binding fragment thereof specific for PD-1 or PD-L1.

13. The method of claim 12, wherein the antibody is an anti-PD-1 antibody, such as cemiplimab, nivolumab, or pembrolizumab.

14. The method of claim 12, wherein the antibody is an anti-PD-L1 antibody, such as avelumab, durvalumab, atezolizumab, KN035, or CK-301.

15. The method of any one of claims 1 to 11, wherein the immune checkpoint inhibitor is a (non-antibody) peptide inhibitor of PD-1/PD-L1, such as AUNP12; a small molecule inhibitor of PD-L1 such as CA-170, or a macrocyclic peptide such as BMS-986189.

16. Use of an IGSF8 antagonist for treating cancer in a subject.

17. The use of claim 16, for combination use with a second therapeutic agent of any one of claims 2 and 12-16.

18. A composition comprising an IGSF8 antagonist for use in any of the preceding method claims 1-15.

19. An antibody which specifically bind IGSF8 for use in a method of treating cancer, preferably through stimulating T cell and/or NK cell activation.

20. An antibody which specifically bind IGSF8 for use in a method of treating cancer, preferably through combination with a second therapeutic agent of any one of claims 2 and 12-16.

21. A monoclonal antibody or an antigen-binding fragment thereof specific for IGSF8, wherein said monoclonal antibody comprises:
(1) a heavy chain variable region (HCVR), comprising HCVR CDR1-CDR3 sequences of any one of antibodies C1-C29, such as C1-C12; and,
(2) a light chain variable region (LCVR), comprising LCVR CDR1-CDR3 sequences of said any one of antibodies C1-C29, such as C1-C12.

22. The monoclonal antibody or antigen-binding fragment thereof of claim 21, comprising:
(a) the HCVR sequence of said any one of antibodies C1-C29, such as C1-C12; and/or,
(b) the LCVR sequence of said any one of antibodies C1-C29, such as C1-C12.

23. The monoclonal antibody or antigen-binding fragment thereof of claim 21 or 22, which is a human-mouse chimeric antibody, a humanized antibody, a human antibody, a CDR-grafted antibody, or a resurfaced antibody.

24. The monoclonal antibody or antigen-binding fragment thereof of any one of claims 21-23, wherein said antigen-binding fragment thereof is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

25. The monoclonal antibody or antigen-binding fragment thereof of any one of claims 21-24, wherein said monoclonal antibody or antigen-binding fragment thereof binds IGSF8 with a K_{d} of less than about 25 nM, 20 nM, 15 nM, 10 nM, 5 nM, 2 nM, or 1 nM.

26. A monoclonal antibody or an antigen-binding fragment thereof, which competes with the monoclonal antibody or antigen-binding fragment thereof of any one of claims 21-25 for binding to IGSF8.

27. A method of stimulating T cell and/or NK cell activation in a tumor microenviroment (TME), the method comprising contacting said T cell and/or NK cell with an IGSF8 (Immuno Globulin Super Family 8) antagonist, such as an antibody or antigen-binding fragment thereof that specifically binds IGSF8.

28. The method of claim 27, further comprising contacting said T cell and/or NK cell with an immune checkpoint inhibitor, such as an antibody or antigen-binding fragment thereof specific for PD-1 or PD-L1.


SEQUENCE LISTING (468 sequences)

SEQ ID NO: 1 - protein, 5 aa, Artificial Sequence
  Description of Artificial Sequence Synthetic peptide
RYRMS

SEQ ID NO: 2 - protein, 17 aa, Artificial Sequence
  Description of Artificial Sequence Synthetic peptide
RISRSGGATAYADSVKG

SEQ ID NO: 3 - protein, 12 aa, Artificial Sequence
  Description of Artificial Sequence Synthetic peptide
DATGRHYNGMDV

SEQ ID NO: 4 - protein, 11 aa, Artificial Sequence
  Description of Artificial Sequence Synthetic peptide
RASQTITRHLN

SEQ ID NO: 5 - protein, 7 aa, Artificial Sequence
  Description of Artificial Sequence Synthetic peptide
GTSALQT

SEQ ID NO: 6 - protein, 9 aa, Artificial Sequence
  Description of Artificial Sequence Synthetic peptide
QQSHTKPWT

SEQ ID NO: 7 - protein, 30 aa, Artificial Sequence
  Description of Artificial Sequence Synthetic polypeptide
QVQLLQSGGGLVQPGGSLRLSCAASGFTFS

SEQ ID NO: 8 - protein, 14 aa, Artificial Sequence
  Description of Artificial Sequence Synthetic peptide
WVRQAPGKGLEWVS

SEQ ID NO: 9 - protein, 32 aa, Artificial Sequence
  Description of Artificial Sequence Synthetic polypeptide
RFTISRDNSKNTLYLQMNSLRAEDTAVYYCAR

SEQ ID NO: 10 - protein, 10 aa, Artificial Sequence
  Description of Artificial Sequence Synthetic peptide
WGRGTLVTVS

SEQ ID NO: 11 - protein, 23 aa, Artificial Sequence
  Description of Artificial Sequence Synthetic peptide
EIALTQSPSSLSASVGDRVTITC

SEQ ID NO: 12 - protein, 15 aa, Artificial Sequence
  Description of Artificial Sequence Synthetic peptide
WFQQKPGKAPNLLIH

SEQ ID NO: 13 - protein, 32 aa, Artificial Sequence
  Description of Artificial Sequence Synthetic polypeptide
GVPPRFSGGGSGTDFTLTINSLQPEDFGTYYC

SEQ ID NO: 14 - protein, 13 aa, Artificial Sequence
  Description of Artificial Sequence Synthetic peptide
FGPGTKVEIKRTV

SEQ ID NO: 15 - protein, 121 aa, Artificial Sequence
  Description of Artificial Sequence Synthetic polypeptide
QVQLLQSGGGLVQPGGSLRLSCAASGFTFSRYRMSWVRQAPGKGLEWVSRISRSGGATAYADSVKGRFTISRDNSKNTLYLQMNSLRAEDTAVYYCARDATGRHYNGMDVWGRGTLVTVSS

SEQ ID NO: 16 - protein, 110 aa, Artificial Sequence
  Description of Artificial Sequence Synthetic polypeptide
EIALTQSPSSLSASVGDRVTITCRASQTITRHLNWFQQKPGKAPNLLIHGTSALQTGVPPRFSGGGSGTDFTLTINSLQPEDFGTYYCQQSHTKPWTFGPGTKVEIKRTV

SEQ ID NO: 17 - protein, 5 aa, Artificial Sequence
  Description of Artificial Sequence Synthetic peptide
SYPMN

SEQ ID NO: 18 - protein, 17 aa, Artificial Sequence
  Description of Artificial Sequence Synthetic peptide
RISRSGGRTSYADSVKG

SEQ ID NO: 19 - protein, 12 aa, Artificial Sequence
  Description of Artificial Sequence Synthetic peptide
DATRRHYNGMDV

SEQ ID NO: 20 - protein, 11 aa, Artificial Sequence
  Description of Artificial Sequence Synthetic peptide
RASRSVGKYLA

SEQ ID NO: 21 - protein, 7 aa, Artificial Sequence
  Description of Artificial Sequence Synthetic peptide
YASLRAG

SEQ ID NO: 22 - protein, 9 aa, Artificial Sequence
  Description of Artificial Sequence Synthetic peptide
QQYGSSPRT

SEQ ID NO: 23 - protein, 30 aa, Artificial Sequence
  Description of Artificial Sequence Synthetic polypeptide
EVQLLQSGGGLVQPGGSLRLSCAASGFTFS

SEQ ID NO: 24 - protein, 14 aa, Artificial Sequence
  Description of Artificial Sequence Synthetic peptide
WVRQAPGKGLEWVS

SEQ ID NO: 25 - protein, 32 aa, Artificial Sequence; identical to SEQ ID NO: 9
  Description of Artificial Sequence Synthetic polypeptide

SEQ ID NO: 26 - protein, 10 aa, Artificial Sequence
  Description of Artificial Sequence Synthetic peptide
WGKGTTVTVS

SEQ ID NO: 27 - protein, 23 aa, Artificial Sequence
  Description of Artificial Sequence Synthetic peptide
DVVMTQSPATLSLSPGERASLSC

SEQ ID NO: 28 - protein, 15 aa, Artificial Sequence
  Description of Artificial Sequence Synthetic peptide
WYQQKPGQAPRLLFY

SEQ ID NO: 29 - protein, 32 aa, Artificial Sequence
  Description of Artificial Sequence Synthetic polypeptide
DIPSRFTASGSGTDFTLTISRLEPEDFAVYYC

SEQ ID NO: 30 - protein, 13 aa, Artificial Sequence
  Description of Artificial Sequence Synthetic peptide
FGQGTKLEMKRTV

SEQ ID NO: 31 - protein, 121 aa, Artificial Sequence
  Description of Artificial Sequence Synthetic polypeptide
EVQLLQSGGGLVQPGGSLRLSCAASGFTFSSYPMNWVRQAPGKGLEWVSRISRSGGRTSYADSVKGRFTISRDNSKNTLYLQMNSLRAEDTAVYYCARDATRRHYNGMDVWGKGTTVTVSS

SEQ ID NO: 32 - protein, 110 aa, Artificial Sequence
  Description of Artificial Sequence Synthetic polypeptide
DVVMTQSPATLSLSPGERASLSCRASRSVGKYLAWYQQKPGQAPRLLFYYASLRAGDIPSRFTASGSGTDFTLTISRLEPEDFAVYYCQQYGSSPRTFGQGTKLEMKRTV

SEQ ID NO: 33 - protein, 5 aa, Artificial Sequence
  Description of Artificial Sequence Synthetic peptide
HYPMR

SEQ ID NO: 34 - protein, 17 aa, Artificial Sequence
  Description of Artificial Sequence Synthetic peptide
SIRRSGGRTKYADSVKG

SEQ ID NO: 35 - protein, 12 aa, Artificial Sequence
  Description of Artificial Sequence Synthetic peptide
DATGRHYNGMDV

SEQ ID NO: 36 - protein, 11 aa, Artificial Sequence
  Description of Artificial Sequence Synthetic peptide
RTSQVIGTSLN

SEQ ID NO: 37 - protein, 7 aa, Artificial Sequence
  Description of Artificial Sequence Synthetic peptide
SASNLQS

SEQ ID NO: 38 - protein, 9 aa, Artificial Sequence
  Description of Artificial Sequence Synthetic peptide
QQSSRVPHT

SEQ ID NO: 39 - protein, 30 aa, Artificial Sequence
  Description of Artificial Sequence Synthetic polypeptide
QVQLVESGGGLVQPGGSLRLSCAASGFTFS

SEQ ID NO: 40 - protein, 14 aa, Artificial Sequence
  Description of Artificial Sequence Synthetic peptide
WVRQAPGKGLEWVS

SEQ ID NO: 41 - protein, 32 aa, Artificial Sequence; identical to SEQ ID NO: 9
  Description of Artificial Sequence Synthetic polypeptide

SEQ ID NO: 42 - protein, 10 aa, Artificial Sequence
  Description of Artificial Sequence Synthetic peptide
WGKGTTVTVS

SEQ ID NO: 43 - protein, 23 aa, Artificial Sequence
  Description of Artificial Sequence Synthetic peptide
DVVMTQSPSSLSASVGDRVTITC

SEQ ID NO: 44 - protein, 15 aa, Artificial Sequence
  Description of Artificial Sequence Synthetic peptide
WYQQKPGRAPRLLIY

SEQ ID NO: 45 - protein, 32 aa, Artificial Sequence
  Description of Artificial Sequence Synthetic polypeptide
GVPSRFSGSGHGTQFTLTISSLQPEDFATYSC

SEQ ID NO: 46 - protein, 13 aa, Artificial Sequence
  Description of Artificial Sequence Synthetic peptide
FGQGTKLEMRRTV

SEQ ID NO: 47 - protein, 121 aa, Artificial Sequence
  Description of Artificial Sequence Synthetic polypeptide
QVQLVESGGGLVQPGGSLRLSCAASGFTFSHYPMRWVRQAPGKGLEWVSSIRRSGGRTKYADSVKGRFTISRDNSKNTLYLQMNSLRAEDTAVYYCARDATGRHYNGMDVWGKGTTVTVSS

SEQ ID NO: 48 - protein, 110 aa, Artificial Sequence
  Description of Artificial Sequence Synthetic polypeptide
DVVMTQSPSSLSASVGDRVTITCRTSQVIGTSLNWYQQKPGRAPRLLIYSASNLQSGVPSRFSGSGHGTQFTLTISSLQPEDFATYSCQQSSRVPHTFGQGTKLEMRRTV

SEQ ID NO: 49 - protein, 5 aa, Artificial Sequence
  Description of Artificial Sequence Synthetic peptide
RYRMG

SEQ ID NO: 50 - protein, 17 aa, Artificial Sequence
  Description of Artificial Sequence Synthetic peptide
SIARSGGRTYYADSVKG

SEQ ID NO: 51 - protein, 20 aa, Artificial Sequence
  Description of Artificial Sequence Synthetic peptide
GVRYCSSPSCSRGPRYAMDV

SEQ ID NO: 52 - protein, 11 aa, Artificial Sequence
  Description of Artificial Sequence Synthetic peptide
RASQGISSWLA

SEQ ID NO: 53 - protein, 7 aa, Artificial Sequence
  Description of Artificial Sequence Synthetic peptide
AASSLQS

SEQ ID NO: 54 - protein, 9 aa, Artificial Sequence
  Description of Artificial Sequence Synthetic peptide
QQANSFPIT

SEQ ID NO: 55 - protein, 30 aa, Artificial Sequence; identical to SEQ ID NO: 7
  Description of Artificial Sequence Synthetic polypeptide

SEQ ID NO: 56 - protein, 14 aa, Artificial Sequence
  Description of Artificial Sequence Synthetic peptide
WVRQAPGKGLEWVS

SEQ ID NO: 57 - protein, 32 aa, Artificial Sequence; identical to SEQ ID NO: 9
  Description of Artificial Sequence Synthetic polypeptide

SEQ ID NO: 58 - protein, 10 aa, Artificial Sequence
  Description of Artificial Sequence Synthetic peptide
WGKGTTVTVS

SEQ ID NO: 59 - protein, 23 aa, Artificial Sequence
  Description of Artificial Sequence Synthetic peptide
EIVMTQSPSSVSASVGDRVTITC

SEQ ID NO: 60 - protein, 15 aa, Artificial Sequence
  Description of Artificial Sequence Synthetic peptide
WYQQKPGKAPKLLIY

SEQ ID NO: 61 - protein, 32 aa, Artificial Sequence
  Description of Artificial Sequence Synthetic polypeptide
GVPSRFSGSGSGTDFTLTISSLQPEDFATYYC

SEQ ID NO: 62 - protein, 13 aa, Artificial Sequence
  Description of Artificial Sequence Synthetic peptide
FGQGTRLEIKRTV

SEQ ID NO: 63 - protein, 129 aa, Artificial Sequence
  Description of Artificial Sequence Synthetic polypeptide
QVQLLQSGGGLVQPGGSLRLSCAASGFTFSRYRMGWVRQAPGKGLEWVSSIARSGGRTYYADSVKGRFTISRDNSKNTLYLQMNSLRAEDTAVYYCARGVRYCSSPSCSRGPRYAMDVWGKGTTVTVSS

SEQ ID NO: 64 - protein, 200 aa, Artificial Sequence
  Description of Artificial Sequence Synthetic polypeptide
EIVMTQSPSSVSASVGDRVTITCRASQGISSWLAWYQQKPGKAPKLLIYAASSLQSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKQTTRNTKSTPAKSPIRA

SEQ ID NO: 65 - protein, 5 aa, Artificial Sequence
  Description of Artificial Sequence Synthetic peptide
RYRMA

SEQ ID NO: 66 - protein, 17 aa, Artificial Sequence
  Description of Artificial Sequence Synthetic peptide
NITRSGGVTRYADSVKG

SEQ ID NO: 67 - protein, 16 aa, Artificial Sequence
  Description of Artificial Sequence Synthetic peptide
DPNRVTAISSHYGMDV

SEQ ID NO: 68 - protein, 11 aa, Artificial Sequence
  Description of Artificial Sequence Synthetic peptide
RASQSISRWLA

SEQ ID NO: 69 - protein, 7 aa, Artificial Sequence
  Description of Artificial Sequence Synthetic peptide
DASNRAT

SEQ ID NO: 70 - protein, 11 aa, Artificial Sequence
  Description of Artificial Sequence Synthetic peptide
QQRSNWPPMYT

SEQ ID NO: 71 - protein, 30 aa, Artificial Sequence
  Description of Artificial Sequence Synthetic polypeptide
EVQLVQSGGGLVQPGGSLRLSCAASGFTFS

SEQ ID NO: 72 - protein, 14 aa, Artificial Sequence
  Description of Artificial Sequence Synthetic peptide
WVRQAPGKGLEWVS

SEQ ID NO: 73 - protein, 32 aa, Artificial Sequence; identical to SEQ ID NO: 9
  Description of Artificial Sequence Synthetic polypeptide

SEQ ID NO: 74 - protein, 10 aa, Artificial Sequence
  Description of Artificial Sequence Synthetic peptide
WGKGTTVTVS

SEQ ID NO: 75 - protein, 23 aa, Artificial Sequence
  Description of Artificial Sequence Synthetic peptide
EIVLTQSPSTLSASVGDRVTISC

SEQ ID NO: 76 - protein, 15 aa, Artificial Sequence
  Description of Artificial Sequence Synthetic peptide
WYQQKPGQAPRLLIY

SEQ ID NO: 77 - protein, 32 aa, Artificial Sequence
  Description of Artificial Sequence Synthetic polypeptide
GVPARFSVSGSETDSTLTISSLEPEDFAMYYC

SEQ ID NO: 78 - protein, 13 aa, Artificial Sequence
  Description of Artificial Sequence Synthetic peptide
FGQGTKLEIKRTV

SEQ ID NO: 79 - protein, 125 aa, Artificial Sequence
  Description of Artificial Sequence Synthetic polypeptide
EVQLVQSGGGLVQPGGSLRLSCAASGFTFSRYRMAWVRQAPGKGLEWVSNITRSGGVTRYADSVKGRFTISRDNSKNTLYLQMNSLRAEDTAVYYCARDPNRVTAISSHYGMDVWGKGTTVTVSS

SEQ ID NO: 80 - protein, 112 aa, Artificial Sequence
  Description of Artificial Sequence Synthetic polypeptide
EIVLTQSPSTLSASVGDRVTISCRASQSISRWLAWYQQKPGQAPRLLIYDASNRATGVPARFSVSGSETDSTLTISSLEPEDFAMYYCQQRSNWPPMYTFGQGTKLEIKRTV

SEQ ID NO: 81 - protein, 5 aa, Artificial Sequence
  Description of Artificial Sequence Synthetic peptide
PYRMH

SEQ ID NO: 82 - protein, 17 aa, Artificial Sequence
  Description of Artificial Sequence Synthetic peptide
RINPSGGRTWYADSVKG

SEQ ID NO: 83 - protein, 12 aa, Artificial Sequence
  Description of Artificial Sequence Synthetic peptide
DATGRHYNGMDV

SEQ ID NO: 84 - protein, 11 aa, Artificial Sequence
  Description of Artificial Sequence Synthetic peptide
RASQSINKWLA

SEQ ID NO: 85 - protein, 7 aa, Artificial Sequence
  Description of Artificial Sequence Synthetic peptide
KASTLES

SEQ ID NO: 86 - protein, 9 aa, Artificial Sequence
  Description of Artificial Sequence Synthetic peptide
QQSHSAPWT

SEQ ID NO: 87 - protein, 30 aa, Artificial Sequence
  Description of Artificial Sequence Synthetic polypeptide
EVQLVESGGGLVQPGGSLRLSCAASGFTFS

SEQ ID NO: 88 - protein, 14 aa, Artificial Sequence
  Description of Artificial Sequence Synthetic peptide
WVRQAPGKGLEWVS

SEQ ID NO: 89 - protein, 32 aa, Artificial Sequence; identical to SEQ ID NO: 9
  Description of Artificial Sequence Synthetic polypeptide

SEQ ID NO: 90 - protein, 11 aa, Artificial Sequence
  Description of Artificial Sequence Synthetic peptide
WGQGTMVTVSS

SEQ ID NO: 91 - protein, 23 aa, Artificial Sequence
  Description of Artificial Sequence Synthetic peptide
DIQMTQSPSTLSASVGDRVTITC

SEQ ID NO: 92 - protein, 15 aa, Artificial Sequence
  Description of Artificial Sequence Synthetic peptide
WYQQKPGKAPKLLIY

SEQ ID NO: 93 - protein, 32 aa, Artificial Sequence
  Description of Artificial Sequence Synthetic polypeptide
GVPSRFSGSGSGTDFTLTINSLQPEDFATYYC

SEQ ID NO: 94 - protein, 13 aa, Artificial Sequence
  Description of Artificial Sequence Synthetic peptide
FGQGTKVEIERTV

SEQ ID NO: 95 - protein, 121 aa, Artificial Sequence
  Description of Artificial Sequence Synthetic polypeptide
EVQLVESGGGLVQPGGSLRLSCAASGFTFSPYRMHWVRQAPGKGLEWVSRINPSGGRTWYADSVKGRFTISRDNSKNTLYLQMNSLRAEDTAVYYCARDATGRHYNGMDVWGQGTMVTVSS

SEQ ID NO: 96 - protein, 110 aa, Artificial Sequence
  Description of Artificial Sequence Synthetic polypeptide
DIQMTQSPSTLSASVGDRVTITCRASQSINKWLAWYQQKPGKAPKLLIYKASTLESGVPSRFSGSGSGTDFTLTINSLQPEDFATYYCQQSHSAPWTFGQGTKVEIERTV

SEQ ID NO: 97 - protein, 5 aa, Artificial Sequence
  Description of Artificial Sequence Synthetic peptide
SYPMN

SEQ ID NO: 98 - protein, 17 aa, Artificial Sequence
  Description of Artificial Sequence Synthetic peptide
RISRSGGRTSYADSVKG

SEQ ID NO: 99 - protein, 12 aa, Artificial Sequence
  Description of Artificial Sequence Synthetic peptide
DATRRHYNGMDV

SEQ ID NO: 100 - protein, 11 aa, Artificial Sequence
  Description of Artificial Sequence Synthetic peptide
RASRSVGKYLA

SEQ ID NO: 101 - protein, 7 aa, Artificial Sequence
  Description of Artificial Sequence Synthetic peptide
YASLRAG

SEQ ID NO: 102 - protein, 9 aa, Artificial Sequence
  Description of Artificial Sequence Synthetic peptide
QQYGSSPRT

SEQ ID NO: 103 - protein, 30 aa, Artificial Sequence; identical to SEQ ID NO: 71
  Description of Artificial Sequence Synthetic polypeptide

SEQ ID NO: 104 - protein, 14 aa, Artificial Sequence
  Description of Artificial Sequence Synthetic peptide
WVRQAPGKGLEWVS

SEQ ID NO: 105 - protein, 32 aa, Artificial Sequence; identical to SEQ ID NO: 9
  Description of Artificial Sequence Synthetic polypeptide

SEQ ID NO: 106 - protein, 10 aa, Artificial Sequence
  Description of Artificial Sequence Synthetic peptide
WGKGTTVTVS

SEQ ID NO: 107 - protein, 23 aa, Artificial Sequence
  Description of Artificial Sequence Synthetic peptide
ETTLTQSPATLSLSPGERASLSC

SEQ ID NO: 108 - protein, 15 aa, Artificial Sequence
  Description of Artificial Sequence Synthetic peptide
WYQQKPGQAPRLLFY

SEQ ID NO: 109 - protein, 32 aa, Artificial Sequence; identical to SEQ ID NO: 29
  Description of Artificial Sequence Synthetic polypeptide

SEQ ID NO: 110 - protein, 13 aa, Artificial Sequence
  Description of Artificial Sequence Synthetic peptide
FGQGTKLEMKRTV

SEQ ID NO: 111 - protein, 121 aa, Artificial Sequence
  Description of Artificial Sequence Synthetic polypeptide
EVQLEESGGGLVQPGGSLRLSCAASGFTFSSYPMNWVRQAPGKGLEWVSRISRSGGRTSYADSVKGRFTISRDNSKNTLYLQMNSLRAEDTAVYYCARDATRRHYNGMDVWGKGTTVTVSS

SEQ ID NO: 112 - protein, 110 aa, Artificial Sequence
  Description of Artificial Sequence Synthetic polypeptide
ETTLTQSPATLSLSPGERASLSCRASRSVGKYLAWYQQKPGQAPRLLFYYASLRAGDIPSRFTASGSGTDFTLTISRLEPEDFAVYYCQQYGSSPRTFGQGTKLEMKRTV

SEQ ID NO: 113 - protein, 5 aa, Artificial Sequence
  Description of Artificial Sequence Synthetic peptide
SYAMS

SEQ ID NO: 114 - protein, 17 aa, Artificial Sequence
  Description of Artificial Sequence Synthetic peptide
AISGSGGSTYYADSVKG

SEQ ID NO: 115 - protein, 15 aa, Artificial Sequence
  Description of Artificial Sequence Synthetic peptide
PYNSAWESYYYGMDV

SEQ ID NO: 116 - protein, 11 aa, Artificial Sequence
  Description of Artificial Sequence Synthetic peptide
RASQGISSRLA

SEQ ID NO: 117 - protein, 7 aa, Artificial Sequence
  Description of Artificial Sequence Synthetic peptide
AASSLQS

SEQ ID NO: 118 - protein, 9 aa, Artificial Sequence
  Description of Artificial Sequence Synthetic peptide
QQRHSYPIT

SEQ ID NO: 119 - protein, 30 aa, Artificial Sequence; identical to SEQ ID NO: 71
  Description of Artificial Sequence Synthetic polypeptide

SEQ ID NO: 120 - protein, 14 aa, Artificial Sequence
  Description of Artificial Sequence Synthetic peptide
WVRQAPGKGLEWVS

SEQ ID NO: 121 - protein, 32 aa, Artificial Sequence; identical to SEQ ID NO: 9
  Description of Artificial Sequence Synthetic polypeptide

SEQ ID NO: 122 - protein, 10 aa, Artificial Sequence
  Description of Artificial Sequence Synthetic peptide
WGKGTTVTVS

SEQ ID NO: 123 - protein, 23 aa, Artificial Sequence
  Description of Artificial Sequence Synthetic peptide
DIQMTQSPSSVSASVGDRVTITC

SEQ ID NO: 124 - protein, 15 aa, Artificial Sequence
  Description of Artificial Sequence Synthetic peptide
WYQQKPGKAPKLLIY

SEQ ID NO: 125 - protein, 32 aa, Artificial Sequence; identical to SEQ ID NO: 61
  Description of Artificial Sequence Synthetic polypeptide

SEQ ID NO: 126 - protein, 13 aa, Artificial Sequence
  Description of Artificial Sequence Synthetic peptide
FGQGTRLEIKRTV

SEQ ID NO: 127 - protein, 124 aa, Artificial Sequence
  Description of Artificial Sequence Synthetic polypeptide
EVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCARPYNSAWESYYYGMDVWGKGTTVTVSS

SEQ ID NO: 128 - protein, 110 aa, Artificial Sequence
  Description of Artificial Sequence Synthetic polypeptide
DIQMTQSPSSVSASVGDRVTITCRASQGISSRLAWYQQKPGKAPKLLIYAASSLQSGVPSRFSGSGSGTDFTLTISSLQPEDFATYYCQQRHSYPITFGQGTRLEIKRTV

SEQ ID NO: 129 - protein, 5 aa, Artificial Sequence
  Description of Artificial Sequence Synthetic peptide
RYDMS

SEQ ID NO: 130 - protein, 17 aa, Artificial Sequence
  Description of Artificial Sequence Synthetic peptide
RIRYSGGRTGYADSVKG

SEQ ID NO: 131 - protein, 20 aa, Artificial Sequence
  Description of Artificial Sequence Synthetic peptide
GVRYCSSPSCSRGPRYAMDV

SEQ ID NO: 132 - protein, 11 aa, Artificial Sequence
  Description of Artificial Sequence Synthetic peptide
RASQSVRGYLA

SEQ ID NO: 133 - protein, 7 aa, Artificial Sequence
  Description of Artificial Sequence Synthetic peptide
DTFKRAT

SEQ ID NO: 134 - protein, 9 aa, Artificial Sequence
  Description of Artificial Sequence Synthetic peptide
QQYFASPWT

SEQ ID NO: 135 - protein, 30 aa, Artificial Sequence; identical to SEQ ID NO: 71
  Description of Artificial Sequence Synthetic polypeptide

SEQ ID NO: 136 - protein, 14 aa, Artificial Sequence
  Description of Artificial Sequence Synthetic peptide
WVRQAPGKGLEWVS

SEQ ID NO: 137 - protein, 32 aa, Artificial Sequence; identical to SEQ ID NO: 9
  Description of Artificial Sequence Synthetic polypeptide

SEQ ID NO: 138 - protein, 10 aa, Artificial Sequence
  Description of Artificial Sequence Synthetic peptide
WGKGTTVTVS

SEQ ID NO: 139 - protein, 23 aa, Artificial Sequence
  Description of Artificial Sequence Synthetic peptide
DVVMTQSPATLSLSPGEGATLSC

SEQ ID NO: 140 - protein, 15 aa, Artificial Sequence
  Description of Artificial Sequence Synthetic peptide
WYQQKPGQAPRLLIY

SEQ ID NO: 141 - protein, 32 aa, Artificial Sequence
  Description of Artificial Sequence Synthetic polypeptide
GIPARFSGSGSGADFTLTISSLEPEDSAVYYC

SEQ ID NO: 142 - protein, 13 aa, Artificial Sequence
  Description of Artificial Sequence Synthetic peptide
FGQGTKVEIKRTV

SEQ ID NO: 143 - protein, 129 aa, Artificial Sequence
  Description of Artificial Sequence Synthetic polypeptide
EVQLVESGGGLVQPGGSLRLSCAASGFTFSRYDMSWVRQAPGKGLEWVSRIRYSGGRTGYADSVKGRFTISRDNSKNTLYLQMNSLRAEDTAVYYCARGVRYCSSPSCSRGPRYAMDVWGKGTTVTVSS

SEQ ID NO: 144 - protein, 110 aa, Artificial Sequence
  Description of Artificial Sequence Synthetic polypeptide
DVVMTQSPATLSLSPGEGATLSCRASQSVRGYLAWYQQKPGQAPRLLIYDTFKRATGIPARFSGSGSGADFTLTISSLEPEDSAVYYCQQYFASPWTFGQGTKVEIKRTV

SEQ ID NO: 145 - protein, 5 aa, Artificial Sequence
  Description of Artificial Sequence Synthetic peptide
RYRMY

SEQ ID NO: 146 - protein, 17 aa, Artificial Sequence
  Description of Artificial Sequence Synthetic peptide
TISRSGGRTVYADSVKG

SEQ ID NO: 147 - protein, 12 aa, Artificial Sequence
  Description of Artificial Sequence Synthetic peptide
DATGRHYNGMDV

SEQ ID NO: 148 - protein, 11 aa, Artificial Sequence
  Description of Artificial Sequence Synthetic peptide
RASQSVSSNVA

SEQ ID NO: 149 - protein, 7 aa, Artificial Sequence
  Description of Artificial Sequence Synthetic peptide
GSGTRAT

SEQ ID NO: 150 - protein, 8 aa, Artificial Sequence
  Description of Artificial Sequence Synthetic peptide
QQYNDWPS

SEQ ID NO: 151 - protein, 30 aa, Artificial Sequence
  Description of Artificial Sequence Synthetic polypeptide
EVQLLESGGGLVQPGGSLRLSCAASGFTFS

SEQ ID NO: 152 - protein, 14 aa, Artificial Sequence
  Description of Artificial Sequence Synthetic peptide
WVRQAPGKGLEWVS

SEQ ID NO: 153 - protein, 32 aa, Artificial Sequence; identical to SEQ ID NO: 9
  Description of Artificial Sequence Synthetic polypeptide

SEQ ID NO: 154 - protein, 9 aa, Artificial Sequence
  Description of Artificial Sequence Synthetic peptide
WGQGTLVTV

SEQ ID NO: 155 - protein, 23 aa, Artificial Sequence
  Description of Artificial Sequence Synthetic peptide
ETTLTQSPATLSVSPGERATLSC

SEQ ID NO: 156 - protein, 15 aa, Artificial Sequence
  Description of Artificial Sequence Synthetic peptide
WYQQKPGQAPRLLMF

SEQ ID NO: 157 - protein, 32 aa, Artificial Sequence
  Description of Artificial Sequence Synthetic polypeptide
GIPARFSGSGSGTEFTLTISSLQSEDFAAYYC

SEQ ID NO: 158 - protein, 13 aa, Artificial Sequence
  Description of Artificial Sequence Synthetic peptide
FGQGTRVEIKGTV

SEQ ID NO: 159 - protein, 119 aa, Artificial Sequence
  Description of Artificial Sequence Synthetic polypeptide
EVQLLESGGGLVQPGGSLRLSCAASGFTFSRYRMYWVRQAPGKGLEWVSTISRSGGRTVYADSVKGRFTISRDNSKNTLYLQMNSLRAEDTAVYYCARDATGRHYNGMDVWGQGTLVTV

SEQ ID NO: 160 - protein, 109 aa, Artificial Sequence
  Description of Artificial Sequence Synthetic polypeptide
ETTLTQSPATLSVSPGERATLSCRASQSVSSNVAWYQQKPGQAPRLLMFGSGTRATGIPARFSGSGSGTEFTLTISSLQSEDFAAYYCQQYNDWPSFGQGTRVEIKGTV

SEQ ID NO: 161 - protein, 5 aa, Artificial Sequence
  Description of Artificial Sequence Synthetic peptide
RYRMY

SEQ ID NO: 162 - protein, 17 aa, Artificial Sequence
  Description of Artificial Sequence Synthetic peptide
SISSSGGRTKYADSVKG

SEQ ID NO: 163 - protein, 20 aa, Artificial Sequence
  Description of Artificial Sequence Synthetic peptide
GVRYCSSPSCSRGPRYAMDV

SEQ ID NO: 164 - protein, 11 aa, Artificial Sequence
  Description of Artificial Sequence Synthetic peptide
RASYVIRNDLS

SEQ ID NO: 165 - protein, 7 aa, Artificial Sequence
  Description of Artificial Sequence Synthetic peptide
GTSSLHN

SEQ ID NO: 166 - protein, 9 aa, Artificial Sequence
  Description of Artificial Sequence Synthetic peptide
LQDDKYPLT

SEQ ID NO: 167 - protein, 30 aa, Artificial Sequence; identical to SEQ ID NO: 71
  Description of Artificial Sequence Synthetic polypeptide

SEQ ID NO: 168 - protein, 14 aa, Artificial Sequence
  Description of Artificial Sequence Synthetic peptide
WVRQAPGKGLEWVS

SEQ ID NO: 169 - protein, 32 aa, Artificial Sequence; identical to SEQ ID NO: 9
  Description of Artificial Sequence Synthetic polypeptide

SEQ ID NO: 170 - protein, 10 aa, Artificial Sequence
  Description of Artificial Sequence Synthetic peptide
WGKGTTVTVS

SEQ ID NO: 171 - protein, 23 aa, Artificial Sequence
  Description of Artificial Sequence Synthetic peptide
DIQMTQSPSSLSASVGDRVTITC

SEQ ID NO: 172 - protein, 15 aa, Artificial Sequence
  Description of Artificial Sequence Synthetic peptide
WYQQKPGKAPKLLIY

SEQ ID NO: 173 - protein, 32 aa, Artificial Sequence
  Description of Artificial Sequence Synthetic polypeptide
GVPSRFSGSGYGTYFTLTISSLQPEDFGTYYC

SEQ ID NO: 174 - protein, 13 aa, Artificial Sequence
  Description of Artificial Sequence Synthetic peptide
FGGGTKVEIKRTV

SEQ ID NO: 175 - protein, 129 aa, Artificial Sequence
  Description of Artificial Sequence Synthetic polypeptide
EVQLVESGGGLVQPGGSLRLSCAASGFTFSRYRMYWVRQAPGKGLEWVSSISSSGGRTKYADSVKGRFTISRDNSKNTLYLQMNSLRAEDTAVYYCARGVRYCSSPSCSRGPRYAMDVWGKGTTVTVSS

SEQ ID NO: 176 - protein, 110 aa, Artificial Sequence
  Description of Artificial Sequence Synthetic polypeptide
DIQMTQSPSSLSASVGDRVTITCRASYVIRNDLSWYQQKPGKAPKLLIYGTSSLHNGVPSRFSGSGYGTYFTLTISSLQPEDFGTYYCLQDDKYPLTFGGGTKVEIKRTV

SEQ ID NO: 177 - protein, 5 aa, Artificial Sequence
  Description of Artificial Sequence Synthetic peptide
KYKMS

SEQ ID NO: 178 - protein, 17 aa, Artificial Sequence
  Description of Artificial Sequence Synthetic peptide
TIAPSGGGTRYADSVKG

SEQ ID NO: 179 - protein, 7 aa, Artificial Sequence
  Description of Artificial Sequence Synthetic peptide
GGHFSNP

SEQ ID NO: 180 - protein, 16 aa, Artificial Sequence
  Description of Artificial Sequence Synthetic peptide
RSSQSLVHTDGDTYLN

SEQ ID NO: 181 - protein, 7 aa, Artificial Sequence
  Description of Artificial Sequence Synthetic peptide
KVSKRDS

SEQ ID NO: 182 - protein, 9 aa, Artificial Sequence
  Description of Artificial Sequence Synthetic peptide
MQGIKRPYT

SEQ ID NO: 183 - protein, 30 aa, Artificial Sequence; identical to SEQ ID NO: 87
  Description of Artificial Sequence Synthetic polypeptide

SEQ ID NO: 184 - protein, 14 aa, Artificial Sequence
  Description of Artificial Sequence Synthetic peptide
WVRQAPGKGLEWVS

SEQ ID NO: 185 - protein, 32 aa, Artificial Sequence; identical to SEQ ID NO: 9
  Description of Artificial Sequence Synthetic polypeptide

SEQ ID NO: 186 - protein, 9 aa, Artificial Sequence
  Description of Artificial Sequence Synthetic peptide
WGQGTLVTV

SEQ ID NO: 187 - protein, 23 aa, Artificial Sequence
  Description of Artificial Sequence Synthetic peptide
DVVMTQSPLSLPVTLGQPASISC

SEQ ID NO: 188 - protein, 15 aa, Artificial Sequence
  Description of Artificial Sequence Synthetic peptide
WYQQRPGQSPRRLIY

SEQ ID NO: 189 - protein, 32 aa, Artificial Sequence
  Description of Artificial Sequence Synthetic polypeptide
GVPDRFSGSGSGTDFTLKISRVEAEDVGVYYC

SEQ ID NO: 190 - protein, 13 aa, Artificial Sequence
  Description of Artificial Sequence Synthetic peptide
LGQGTKLEIKRTV

SEQ ID NO: 191 - protein, 116 aa, Artificial Sequence
  Description of Artificial Sequence Synthetic polypeptide
EVQLVESGGGLVQPGGSLRLSCAASGFTFSKYKMSWVRQAPGKGLEWVSTIAPSGGGTRYADSVKGRFTISRDNSKNTLYLQMNSLRAEDTAVYYCARGGHFSNPWGQGTLVTVSS

SEQ ID NO: 192 - protein, 219 aa, Artificial Sequence
  Description of Artificial Sequence Synthetic polypeptide
DVVMTQSPLSLPVTLGQPASISCRSSQSLVHTDGDTYLNWYQQRPGQSPRRLIYKVSKRDSGVPDRFSGSGSGTDFTLKISRVEAEDVGVYYCMQGIKRPYTLGQGTKLEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 193 - protein, 5 aa, Artificial Sequence
  Description of Artificial Sequence Synthetic peptide
PYRMH

SEQ ID NO: 194 - protein, 17 aa, Artificial Sequence
  Description of Artificial Sequence Synthetic peptide
SINRSGGRTNYADSVKG

SEQ ID NO: 195 - protein, 9 aa, Artificial Sequence
  Description of Artificial Sequence Synthetic peptide
GRGIGTFRN

SEQ ID NO: 196 - protein, 11 aa, Artificial Sequence
  Description of Artificial Sequence Synthetic peptide
RASQSVSTYLA

SEQ ID NO: 197 - protein, 7 aa, Artificial Sequence
  Description of Artificial Sequence Synthetic peptide
DASNRAT

SEQ ID NO: 198 - protein, 9 aa, Artificial Sequence
  Description of Artificial Sequence Synthetic peptide
QQRNNWPPT

SEQ ID NO: 199 - protein, 30 aa, Artificial Sequence; identical to SEQ ID NO: 87
  Description of Artificial Sequence Synthetic polypeptide

SEQ ID NO: 200 - protein, 14 aa, Artificial Sequence
  Description of Artificial Sequence Synthetic peptide
WVRQAPGKGLEWVS

SEQ ID NO: 201 - protein, 32 aa, Artificial Sequence
  Description of Artificial Sequence Synthetic polypeptide
RFTISRDNSKNTLYLQMNSLRAEDTAVYYCAT

SEQ ID NO: 202 - protein, 11 aa, Artificial Sequence
  Description of Artificial Sequence Synthetic peptide
WGQGTLVTVSS

SEQ ID NO: 203 - protein, 23 aa, Artificial Sequence
  Description of Artificial Sequence Synthetic peptide
DIALTQSPATLSLSPGERATLSC

SEQ ID NO: 204 - protein, 15 aa, Artificial Sequence
  Description of Artificial Sequence Synthetic peptide
WYQQKPGQAPRLLIS

SEQ ID NO: 205 - protein, 32 aa, Artificial Sequence
  Description of Artificial Sequence Synthetic polypeptide
GIPARFSGSGSGTDFTLTISSLEPEDFAVYYC

SEQ ID NO: 206 - protein, 13 aa, Artificial Sequence
  Description of Artificial Sequence Synthetic peptide
FGQGTKVEIKRTV

SEQ ID NO: 207 - protein, 118 aa, Artificial Sequence
  Description of Artificial Sequence Synthetic polypeptide
EVQLVESGGGLVQPGGSLRLSCAASGFTFSPYRMHWVRQAPGKGLEWVSSINRSGGRTNYADSVKGRFTISRDNSKNTLYLQMNSLRAEDTAVYYCATGRGIGTFRNWGQGTLVTVSS

SEQ ID NO: 208 - protein, 214 aa, Artificial Sequence
  Description of Artificial Sequence Synthetic polypeptide
DIALTQSPATLSLSPGERATLSCRASQSVSTYLAWYQQKPGQAPRLLISDASNRATGIPARFSGSGSGTDFTLTISSLEPEDFAVYYCQQRNNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 209 - protein, 5 aa, Artificial Sequence
  Description of Artificial Sequence Synthetic peptide
SYAMS

SEQ ID NO: 210 - protein, 17 aa, Artificial Sequence
  Description of Artificial Sequence Synthetic peptide
AISGSGGSTYYADSVKG

SEQ ID NO: 211 - protein, 9 aa, Artificial Sequence
  Description of Artificial Sequence Synthetic peptide
DTIPGYMDV

SEQ ID NO: 212 - protein, 11 aa, Artificial Sequence
  Description of Artificial Sequence Synthetic peptide
RASQSISNYLS

SEQ ID NO: 213 - protein, 7 aa, Artificial Sequence
  Description of Artificial Sequence Synthetic peptide
AASSLQS

SEQ ID NO: 214 - protein, 9 aa, Artificial Sequence
  Description of Artificial Sequence Synthetic peptide
QQSYSSPYT

SEQ ID NO: 215 - protein, 30 aa, Artificial Sequence; identical to SEQ ID NO: 151
  Description of Artificial Sequence Synthetic polypeptide

SEQ ID NO: 216 - protein, 14 aa, Artificial Sequence
  Description of Artificial Sequence Synthetic peptide
WVRQAPGKGLEWVS

SEQ ID NO: 217 - protein, 32 aa, Artificial Sequence; identical to SEQ ID NO: 9
  Description of Artificial Sequence Synthetic polypeptide

SEQ ID NO: 218 - protein, 9 aa, Artificial Sequence
  Description of Artificial Sequence Synthetic peptide
WGQGTLVTV

SEQ ID NO: 219 - protein, 23 aa, Artificial Sequence
  Description of Artificial Sequence Synthetic peptide
DIMLTQSPSSLSGSVGDSVTFTC

SEQ ID NO: 220 - protein, 15 aa, Artificial Sequence
  Description of Artificial Sequence Synthetic peptide
WYQQKSGKAPQLLIY

SEQ ID NO: 221 - protein, 32 aa, Artificial Sequence; identical to SEQ ID NO: 61
  Description of Artificial Sequence Synthetic polypeptide

SEQ ID NO: 222 - protein, 13 aa, Artificial Sequence
  Description of Artificial Sequence Synthetic peptide
FGQGTKLEIKRTV

SEQ ID NO: 223 - protein, 118 aa, Artificial Sequence
  Description of Artificial Sequence Synthetic polypeptide
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARDTIPGYMDVWGKGTTVTVSS

SEQ ID NO: 224 - protein, 214 aa, Artificial Sequence
  Description of Artificial Sequence Synthetic polypeptide
DIMLTQSPSSLSGSVGDSVTFTCRASQSISNYLSWYQQKSGKAPQLLIYAASSLQSGVPSRFSGSGSGTDFTLTISSLQPEDFATYYCQQSYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 225 - protein, 5 aa, Artificial Sequence
  Description of Artificial Sequence Synthetic peptide
RYRMA

SEQ ID NO: 226 - protein, 17 aa, Artificial Sequence
  Description of Artificial Sequence Synthetic peptide
AIARSGGRTWYADSVKG

SEQ ID NO: 227 - protein, 13 aa, Artificial Sequence
  Description of Artificial Sequence Synthetic peptide
GGGAKWLYNWFDS

SEQ ID NO: 228 - protein, 12 aa, Artificial Sequence
  Description of Artificial Sequence Synthetic peptide
RASQSVSNTYLA

SEQ ID NO: 229 - protein, 7 aa, Artificial Sequence
  Description of Artificial Sequence Synthetic peptide
GASIRAP

SEQ ID NO: 230 - protein, 9 aa, Artificial Sequence
  Description of Artificial Sequence Synthetic peptide
QQYARSRIA

SEQ ID NO: 231 - protein, 30 aa, Artificial Sequence; identical to SEQ ID NO: 87
  Description of Artificial Sequence Synthetic polypeptide

SEQ ID NO: 232 - protein, 14 aa, Artificial Sequence
  Description of Artificial Sequence Synthetic peptide
WVRQAPGKGLEWVS

SEQ ID NO: 233 - protein, 32 aa, Artificial Sequence; identical to SEQ ID NO: 9
  Description of Artificial Sequence Synthetic polypeptide

SEQ ID NO: 234 - protein, 9 aa, Artificial Sequence
  Description of Artificial Sequence Synthetic peptide
WGQGTLVTV

SEQ ID NO: 235 - protein, 23 aa, Artificial Sequence
  Description of Artificial Sequence Synthetic peptide
EIVLTQSPGTLSLSAGERATLSC

SEQ ID NO: 236 - protein, 15 aa, Artificial Sequence
  Description of Artificial Sequence Synthetic peptide
WYQQKPGQAPRLLIY

SEQ ID NO: 237 - protein, 32 aa, Artificial Sequence
  Description of Artificial Sequence Synthetic polypeptide
GIPDRFSGSGSGTDFTLTVNRLEPEDSAVYYC

SEQ ID NO: 238 - protein, 13 aa, Artificial Sequence
  Description of Artificial Sequence Synthetic peptide
FGQGTRLEIRRTV

SEQ ID NO: 239 - protein, 195 aa, Artificial Sequence
  Description of Artificial Sequence Synthetic polypeptide
LRGGISRARLVNRQIAWRRHPRCFDLHRRHRDRSSLRTRPQTTRQTCKRRHAQLSTALLPGPPDWGEGPGAAGAVGVLLTGVRAEVQLVESGGGLVQPGGSLRLSCAASGFTFSRYRMAWVRQAPGKGLEWVSAIARSGGRTWYADSVKGRFTISRDNSKNTLYLQMNSLRAEDTAVYYCARGGGAKWLYNWFDS

SEQ ID NO: 240 - protein, 111 aa, Artificial Sequence
  Description of Artificial Sequence Synthetic polypeptide
EIVLTQSPGTLSLSAGERATLSCRASQSVSNTYLAWYQQKPGQAPRLLIYGASIRAPGIPDRFSGSGSGTDFTLTVNRLEPEDSAVYYCQQYARSRIAFGQGTRLEIRRTV

SEQ ID NO: 241 - protein, 5 aa, Artificial Sequence
  Description of Artificial Sequence Synthetic peptide
HYWMG

SEQ ID NO: 242 - protein, 17 aa, Artificial Sequence
  Description of Artificial Sequence Synthetic peptide
GIGASGGWTGYADSVKG

SEQ ID NO: 243 - protein, 8 aa, Artificial Sequence
  Description of Artificial Sequence Synthetic peptide
TSGAYFDY

SEQ ID NO: 244 - protein, 12 aa, Artificial Sequence
  Description of Artificial Sequence Synthetic peptide
RASQSVSSDYLA

SEQ ID NO: 245 - protein, 7 aa, Artificial Sequence
  Description of Artificial Sequence Synthetic peptide
GASSRAT

SEQ ID NO: 246 - protein, 9 aa, Artificial Sequence
  Description of Artificial Sequence Synthetic peptide
QQYGSTPLT

SEQ ID NO: 247 - protein, 30 aa, Artificial Sequence; identical to SEQ ID NO: 151
  Description of Artificial Sequence Synthetic polypeptide

SEQ ID NO: 248 - protein, 14 aa, Artificial Sequence
  Description of Artificial Sequence Synthetic peptide
WVRQAPGKGLEWVS

SEQ ID NO: 249 - protein, 32 aa, Artificial Sequence; identical to SEQ ID NO: 9
  Description of Artificial Sequence Synthetic polypeptide

SEQ ID NO: 250 - protein, 9 aa, Artificial Sequence
  Description of Artificial Sequence Synthetic peptide
WGQGTLVTV

SEQ ID NO: 251 - protein, 23 aa, Artificial Sequence
  Description of Artificial Sequence Synthetic peptide
EIVLTQSPGTLSLSPGQRATLSC

SEQ ID NO: 252 - protein, 15 aa, Artificial Sequence
  Description of Artificial Sequence Synthetic peptide
WYQQKPGQAPRLLMY

SEQ ID NO: 253 - protein, 32 aa, Artificial Sequence
  Description of Artificial Sequence Synthetic polypeptide
GIPDRFSGSGSGTDFTLTISRLEPEDFAVYYC

SEQ ID NO: 254 - protein, 13 aa, Artificial Sequence
  Description of Artificial Sequence Synthetic peptide
FGGGTTVEIRRTV

SEQ ID NO: 255 - protein, 117 aa, Artificial Sequence
  Description of Artificial Sequence Synthetic polypeptide
EVQLVESGGGLVQPGGSLRLSCAASGFTFSHYWMGWVRQAPGKGLEWVSGIGASGGWTGYADSVKGRFTISRDNSKNTLYLQMNSLRAEDTAVYYCARTSGAYFDYWGQGTLVTVSS

SEQ ID NO: 256 - protein, 111 aa, Artificial Sequence
  Description of Artificial Sequence Synthetic polypeptide
EIVLTQSPGTLSLSPGQRATLSCRASQSVSSDYLAWYQQKPGQAPRLLMYGASSRATGIPDRFSGSGSGTDFTLTISRLEPEDFAVYYCQQYGSTPLTFGGGTTVEIRRTV

SEQ ID NO: 257 - protein, 5 aa, Artificial Sequence
  Description of Artificial Sequence Synthetic peptide
NYPMT

SEQ ID NO: 258 - protein, 17 aa, Artificial Sequence
  Description of Artificial Sequence Synthetic peptide
TIRGSGGDTWYADSVKG

SEQ ID NO: 259 - protein, 6 aa, Artificial Sequence
  Description of Artificial Sequence Synthetic peptide
WVGRDA

SEQ ID NO: 260 - protein, 16 aa, Artificial Sequence
  Description of Artificial Sequence Synthetic peptide
RSSQSLVYSDGNTYLN

SEQ ID NO: 261 - protein, 7 aa, Artificial Sequence
  Description of Artificial Sequence Synthetic peptide
KVSNRDS

SEQ ID NO: 262 - protein, 9 aa, Artificial Sequence
  Description of Artificial Sequence Synthetic peptide
MQGTHWPPT

SEQ ID NO: 263 - protein, 30 aa, Artificial Sequence; identical to SEQ ID NO: 151
  Description of Artificial Sequence Synthetic polypeptide

SEQ ID NO: 264 - protein, 14 aa, Artificial Sequence
  Description of Artificial Sequence Synthetic peptide
WVRQAPGKGLEWVS

SEQ ID NO: 265 - protein, 32 aa, Artificial Sequence; identical to SEQ ID NO: 9
  Description of Artificial Sequence Synthetic polypeptide

SEQ ID NO: 266 - protein, 9 aa, Artificial Sequence
  Description of Artificial Sequence Synthetic peptide
WGQGTLVTV

SEQ ID NO: 267 - protein, 23 aa, Artificial Sequence
  Description of Artificial Sequence Synthetic peptide
DIVLTQSPLSLPVTLGQPASISC

SEQ ID NO: 268 - protein, 15 aa, Artificial Sequence
  Description of Artificial Sequence Synthetic peptide
WFRQRPGQSPRRLIY

SEQ ID NO: 269 - protein, 32 aa, Artificial Sequence
  Description of Artificial Sequence Synthetic polypeptide
GVPDRFSGSGSGTDFTLRISRVEAEDVGVYYC

SEQ ID NO: 270 - protein, 13 aa, Artificial Sequence
  Description of Artificial Sequence Synthetic peptide
FGQGTKLEIKRTV

SEQ ID NO: 271 - protein, 115 aa, Artificial Sequence
  Description of Artificial Sequence Synthetic polypeptide
EVQLVESGGGLVQPGGSLRLSCAASGFTFSNYPMTWVRQAPGKGLEWVSTIRGSGGDTWYADSVKGRFTISRDNSKNTLYLQMNSLRAEDTAVYYCAKWVGRDAWGQGTLVTVSS

SEQ ID NO: 272 - protein, 115 aa, Artificial Sequence
  Description of Artificial Sequence Synthetic polypeptide
DIVLTQSPLSLPVTLGQPASISCRSSQSLVYSDGNTYLNWFRQRPGQSPRRLIYKVSNRDSGVPDRFSGSGSGTDFTLRISRVEAEDVGVYYCMQGTHWPPTFGQGTKLEIKRTV

SEQ ID NO: 273 - protein, 5 aa, Artificial Sequence
  Description of Artificial Sequence Synthetic peptide
SYPMN

SEQ ID NO: 274 - protein, 17 aa, Artificial Sequence
  Description of Artificial Sequence Synthetic peptide
RISRSGGRTSYADSVKG

SEQ ID NO: 275 - protein, 12 aa, Artificial Sequence
  Description of Artificial Sequence Synthetic peptide
DATRRHYNGMDV

SEQ ID NO: 276 - protein, 11 aa, Artificial Sequence
  Description of Artificial Sequence Synthetic peptide
RASRSVGKYLA

SEQ ID NO: 277 - protein, 7 aa, Artificial Sequence
  Description of Artificial Sequence Synthetic peptide
YASLRAG

SEQ ID NO: 278 - protein, 9 aa, Artificial Sequence
  Description of Artificial Sequence Synthetic peptide
QQYGSSPRT

SEQ ID NO: 279 - protein, 30 aa, Artificial Sequence; identical to SEQ ID NO: 87
  Description of Artificial Sequence Synthetic polypeptide

SEQ ID NO: 280 - protein, 14 aa, Artificial Sequence
  Description of Artificial Sequence Synthetic peptide
WVRQAPGKGLEWVS

SEQ ID NO: 281 - protein, 32 aa, Artificial Sequence; identical to SEQ ID NO: 9
  Description of Artificial Sequence Synthetic polypeptide

SEQ ID NO: 282 - protein, 11 aa, Artificial Sequence
  Description of Artificial Sequence Synthetic peptide
WGKGTTVTVSS

SEQ ID NO: 283 - protein, 23 aa, Artificial Sequence
  Description of Artificial Sequence Synthetic peptide
DIVLTQSPATLSLSPGERASLSC

SEQ ID NO: 284 - protein, 15 aa, Artificial Sequence
  Description of Artificial Sequence Synthetic peptide
WYQQKPGQAPRLLFY

SEQ ID NO: 285 - protein, 32 aa, Artificial Sequence; identical to SEQ ID NO: 29
  Description of Artificial Sequence Synthetic polypeptide

SEQ ID NO: 286 - protein, 13 aa, Artificial Sequence
  Description of Artificial Sequence Synthetic peptide
FGQGTKLEMKRTV

SEQ ID NO: 287 - protein, 121 aa, Artificial Sequence
  Description of Artificial Sequence Synthetic polypeptide
EVQLVESGGGLVQPGGSLRLSCAASGFTFSSYPMNWVRQAPGKGLEWVSRISRSGGRTSYADSVKGRFTISRDNSKNTLYLQMNSLRAEDTAVYYCARDATRRHYNGMDVWGKGTTVTVSS

SEQ ID NO: 288 - protein, 110 aa, Artificial Sequence
  Description of Artificial Sequence Synthetic polypeptide
DIVLTQSPATLSLSPGERASLSCRASRSVGKYLAWYQQKPGQAPRLLFYYASLRAGDIPSRFTASGSGTDFTLTISRLEPEDFAVYYCQQYGSSPRTFGQGTKLEMKRTV

SEQ ID NO: 289 - protein, 5 aa, Artificial Sequence
  Description of Artificial Sequence Synthetic peptide
RYRMH

SEQ ID NO: 290 - protein, 17 aa, Artificial Sequence
  Description of Artificial Sequence Synthetic peptide
SIASSGGRTRYADSVKG

SEQ ID NO: 291 - protein, 13 aa, Artificial Sequence
  Description of Artificial Sequence Synthetic peptide
GGLPYRGHYGMDV

SEQ ID NO: 292 - protein, 11 aa, Artificial Sequence
  Description of Artificial Sequence Synthetic peptide
RASQSISSYLN

SEQ ID NO: 293 - protein, 7 aa, Artificial Sequence
  Description of Artificial Sequence Synthetic peptide
VASSLQS

SEQ ID NO: 294 - protein, 9 aa, Artificial Sequence
  Description of Artificial Sequence Synthetic peptide
QQARSIPWT

SEQ ID NO: 295 - protein, 30 aa, Artificial Sequence; identical to SEQ ID NO: 87
  Description of Artificial Sequence Synthetic polypeptide

SEQ ID NO: 296 - protein, 14 aa, Artificial Sequence
  Description of Artificial Sequence Synthetic peptide
WVRQAPGKGLEWVS

SEQ ID NO: 297 - protein, 32 aa, Artificial Sequence; identical to SEQ ID NO: 9
  Description of Artificial Sequence Synthetic polypeptide

SEQ ID NO: 298 - protein, 11 aa, Artificial Sequence
  Description of Artificial Sequence Synthetic peptide
WGQGTLVTVSS

SEQ ID NO: 299 - protein, 23 aa, Artificial Sequence
  Description of Artificial Sequence Synthetic peptide
EIMLTQSPSSLSASVGDRVTITC

SEQ ID NO: 300 - protein, 15 aa, Artificial Sequence
  Description of Artificial Sequence Synthetic peptide
WYQQKPGKAPKLLIS

SEQ ID NO: 301 - protein, 32 aa, Artificial Sequence
  Description of Artificial Sequence Synthetic polypeptide
GVPSRFSGSRSGTDFTLTISSLQPEDFATYYC

SEQ ID NO: 302 - protein, 13 aa, Artificial Sequence
  Description of Artificial Sequence Synthetic peptide
FGQGTNVEIKRTV

SEQ ID NO: 303 - protein, 122 aa, Artificial Sequence
  Description of Artificial Sequence Synthetic polypeptide
EVQLVESGGGLVQPGGSLRLSCAASGFTFSRYRMHWVRQAPGKGLEWVSSIASSGGRTRYADSVKGRFTISRDNSKNTLYLQMNSLRAEDTAVYYCARGGLPYRGHYGMDVWGQGTLVTVSS

SEQ ID NO: 304 - protein, 110 aa, Artificial Sequence
  Description of Artificial Sequence Synthetic polypeptide
EIMLTQSPSSLSASVGDRVTITCRASQSISSYLNWYQQKPGKAPKLLISVASSLQSGVPSRFSGSRSGTDFTLTISSLQPEDFATYYCQQARSIPWTFGQGTNVEIKRTV

SEQ ID NO: 305 - protein, 5 aa, Artificial Sequence
  Description of Artificial Sequence Synthetic peptide
SYAMS

SEQ ID NO: 306 - protein, 17 aa, Artificial Sequence
  Description of Artificial Sequence Synthetic peptide
AISGSGGSTYYADSVKG

SEQ ID NO: 307 - protein, 13 aa, Artificial Sequence
  Description of Artificial Sequence Synthetic peptide
GGLPYRGHYGMDV

SEQ ID NO: 308 - protein, 16 aa, Artificial Sequence
  Description of Artificial Sequence Synthetic peptide
RSSQSLLHSNGYNYVD

SEQ ID NO: 309 - protein, 7 aa, Artificial Sequence
  Description of Artificial Sequence Synthetic peptide
LGSNRAS

SEQ ID NO: 310 - protein, 9 aa, Artificial Sequence
  Description of Artificial Sequence Synthetic peptide
MQALKIPRT

SEQ ID NO: 311 - protein, 30 aa, Artificial Sequence; identical to SEQ ID NO: 87
  Description of Artificial Sequence Synthetic polypeptide

SEQ ID NO: 312 - protein, 14 aa, Artificial Sequence
  Description of Artificial Sequence Synthetic peptide
WVRQAPGKGLEWVS

SEQ ID NO: 313 - protein, 32 aa, Artificial Sequence; identical to SEQ ID NO: 9
  Description of Artificial Sequence Synthetic polypeptide

SEQ ID NO: 314 - protein, 11 aa, Artificial Sequence
  Description of Artificial Sequence Synthetic peptide
WGQGTLVTVSS

SEQ ID NO: 315 - protein, 23 aa, Artificial Sequence
  Description of Artificial Sequence Synthetic peptide
DIVLTQSPLSLPVTPGEPASISC

SEQ ID NO: 316 - protein, 15 aa, Artificial Sequence
  Description of Artificial Sequence Synthetic peptide
WYLQKPGQSPQLLIY

SEQ ID NO: 317 - protein, 32 aa, Artificial Sequence; identical to SEQ ID NO: 189
  Description of Artificial Sequence Synthetic polypeptide

SEQ ID NO: 318 - protein, 13 aa, Artificial Sequence
  Description of Artificial Sequence Synthetic peptide
FGQGTKVEIKRTV

SEQ ID NO: 319 - protein, 122 aa, Artificial Sequence
  Description of Artificial Sequence Synthetic polypeptide
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARGGLPYRGHYGMDVWGQGTLVTVSS

SEQ ID NO: 320 - protein, 115 aa, Artificial Sequence
  Description of Artificial Sequence Synthetic polypeptide
DIVLTQSPLSLPVTPGEPASISCRSSQSLLHSNGYNYVDWYLQKPGQSPQLLIYLGSNRASGVPDRFSGSGSGTDFTLKISRVEAEDVGVYYCMQALKIPRTFGQGTKVEIKRTV

SEQ ID NO: 321 - protein, 5 aa, Artificial Sequence
  Description of Artificial Sequence Synthetic peptide
PYYMV

SEQ ID NO: 322 - protein, 17 aa, Artificial Sequence
  Description of Artificial Sequence Synthetic peptide
SINRSGGRTAYADSVKG

SEQ ID NO: 323 - protein, 11 aa, Artificial Sequence
  Description of Artificial Sequence Synthetic peptide
AIAAGRYGMDV

SEQ ID NO: 324 - protein, 11 aa, Artificial Sequence
  Description of Artificial Sequence Synthetic peptide
RASQSVSSYLA

SEQ ID NO: 325 - protein, 7 aa, Artificial Sequence
  Description of Artificial Sequence Synthetic peptide
DASNRAT

SEQ ID NO: 326 - protein, 10 aa, Artificial Sequence
  Description of Artificial Sequence Synthetic peptide
QQRTNWPPLT

SEQ ID NO: 327 - protein, 30 aa, Artificial Sequence; identical to SEQ ID NO: 87
  Description of Artificial Sequence Synthetic polypeptide

SEQ ID NO: 328 - protein, 14 aa, Artificial Sequence
  Description of Artificial Sequence Synthetic peptide
WVRQAPGKGLEWVS

SEQ ID NO: 329 - protein, 32 aa, Artificial Sequence; identical to SEQ ID NO: 9
  Description of Artificial Sequence Synthetic polypeptide

SEQ ID NO: 330 - protein, 11 aa, Artificial Sequence
  Description of Artificial Sequence Synthetic peptide
WGQGTLVTVSS

SEQ ID NO: 331 - protein, 23 aa, Artificial Sequence
  Description of Artificial Sequence Synthetic peptide
DIEMTQSPATLSLSPGERATLSC

SEQ ID NO: 332 - protein, 15 aa, Artificial Sequence
  Description of Artificial Sequence Synthetic peptide
WYQQKPGQPPRLLIY

SEQ ID NO: 333 - protein, 32 aa, Artificial Sequence; identical to SEQ ID NO: 205
  Description of Artificial Sequence Synthetic polypeptide

SEQ ID NO: 334 - protein, 13 aa, Artificial Sequence
  Description of Artificial Sequence Synthetic peptide
FGGGTKVEIKRTV

SEQ ID NO: 335 - protein, 120 aa, Artificial Sequence
  Description of Artificial Sequence Synthetic polypeptide
EVQLVESGGGLVQPGGSLRLSCAASGFTFSPYYMVWVRQAPGKGLEWVSSINRSGGRTAYADSVKGRFTISRDNSKNTLYLQMNSLRAEDTAVYYCARAIAAGRYGMDVWGKGTTVTVSS

SEQ ID NO: 336 - protein, 111 aa, Artificial Sequence
  Description of Artificial Sequence Synthetic polypeptide
DIEMTQSPATLSLSPGERATLSCRASQSVSSYLAWYQQKPGQPPRLLIYDASNRATGIPARFSGSGSGTDFTLTISSLEPEDFAVYYCQQRTNWPPLTFGGGTKVEIKRTV

SEQ ID NO: 337 - protein, 5 aa, Artificial Sequence
  Description of Artificial Sequence Synthetic peptide
RYTMR

SEQ ID NO: 338 - protein, 17 aa, Artificial Sequence
  Description of Artificial Sequence Synthetic peptide
GISRSGGRTVYADSVKG

SEQ ID NO: 339 - protein, 14 aa, Artificial Sequence
  Description of Artificial Sequence Synthetic peptide
DPFGVVNHFYYMDV

SEQ ID NO: 340 - protein, 11 aa, Artificial Sequence
  Description of Artificial Sequence Synthetic peptide
RASQSIHTYLN

SEQ ID NO: 341 - protein, 7 aa, Artificial Sequence
  Description of Artificial Sequence Synthetic peptide
GASNLQN

SEQ ID NO: 342 - protein, 9 aa, Artificial Sequence
  Description of Artificial Sequence Synthetic peptide
QQTYRTPTT

SEQ ID NO: 343 - protein, 30 aa, Artificial Sequence; identical to SEQ ID NO: 87
  Description of Artificial Sequence Synthetic polypeptide

SEQ ID NO: 344 - protein, 14 aa, Artificial Sequence
  Description of Artificial Sequence Synthetic peptide
WVRQAPGKGLEWVS

SEQ ID NO: 345 - protein, 32 aa, Artificial Sequence; identical to SEQ ID NO: 9
  Description of Artificial Sequence Synthetic polypeptide

SEQ ID NO: 346 - protein, 11 aa, Artificial Sequence
  Description of Artificial Sequence Synthetic peptide
WGKGTTVTVSS

SEQ ID NO: 347 - protein, 23 aa, Artificial Sequence
  Description of Artificial Sequence Synthetic peptide
EIMLTQSPPSLSASVGDRVTITC

SEQ ID NO: 348 - protein, 15 aa, Artificial Sequence
  Description of Artificial Sequence Synthetic peptide
WYQQKPGKAPKLLIY

SEQ ID NO: 349 - protein, 32 aa, Artificial Sequence
  Description of Artificial Sequence Synthetic polypeptide
GVPSRFSGTGSGTDFALTISSLQPEDFATYSC

SEQ ID NO: 350 - protein, 13 aa, Artificial Sequence
  Description of Artificial Sequence Synthetic peptide
FGPGTKVDIKRTV

SEQ ID NO: 351 - protein, 123 aa, Artificial Sequence
  Description of Artificial Sequence Synthetic polypeptide
EVQLVESGGGLVQPGGSLRLSCAASGFTFSRYTMRWVRQAPGKGLEWVSGISRSGGRTVYADSVKGRFTISRDNSKNTLYLQMNSLRAEDTAVYYCARDPFGVVNHFYYMDVWGKGTTVTVSS

SEQ ID NO: 352 - protein, 110 aa, Artificial Sequence
  Description of Artificial Sequence Synthetic polypeptide
EIMLTQSPPSLSASVGDRVTITCRASQSIHTYLNWYQQKPGKAPKLLIYGASNLQNGVPSRFSGTGSGTDFALTISSLQPEDFATYSCQQTYRTPTTFGPGTKVDIKRTV

SEQ ID NO: 353 - protein, 5 aa, Artificial Sequence
  Description of Artificial Sequence Synthetic peptide
SYRMS

SEQ ID NO: 354 - protein, 17 aa, Artificial Sequence
  Description of Artificial Sequence Synthetic peptide
GIGRSGGRTRYADSVKG

SEQ ID NO: 355 - protein, 11 aa, Artificial Sequence
  Description of Artificial Sequence Synthetic peptide
AIAAGRYGMDV

SEQ ID NO: 356 - protein, 12 aa, Artificial Sequence
  Description of Artificial Sequence Synthetic peptide
RASQSIRNNYLA

SEQ ID NO: 357 - protein, 7 aa, Artificial Sequence
  Description of Artificial Sequence Synthetic peptide
GASYRAT

SEQ ID NO: 358 - protein, 9 aa, Artificial Sequence
  Description of Artificial Sequence Synthetic peptide
QQRSNWPPT

SEQ ID NO: 359 - protein, 30 aa, Artificial Sequence; identical to SEQ ID NO: 87
  Description of Artificial Sequence Synthetic polypeptide

SEQ ID NO: 360 - protein, 14 aa, Artificial Sequence
  Description of Artificial Sequence Synthetic peptide
WVRQAPGKGLEWVS

SEQ ID NO: 361 - protein, 32 aa, Artificial Sequence; identical to SEQ ID NO: 9
  Description of Artificial Sequence Synthetic polypeptide

SEQ ID NO: 362 - protein, 11 aa, Artificial Sequence
  Description of Artificial Sequence Synthetic peptide
WGKGTTVTVSS

SEQ ID NO: 363 - protein, 23 aa, Artificial Sequence
  Description of Artificial Sequence Synthetic peptide
DIMLTQSPGTLSLSPGERATLSC

SEQ ID NO: 364 - protein, 15 aa, Artificial Sequence
  Description of Artificial Sequence Synthetic peptide
WYQQRPGQAPRLLIY

SEQ ID NO: 365 - protein, 32 aa, Artificial Sequence
  Description of Artificial Sequence Synthetic polypeptide
GIPDRFSGSGSGTDFTLTISSLEPEDFAVYYC

SEQ ID NO: 366 - protein, 13 aa, Artificial Sequence
  Description of Artificial Sequence Synthetic peptide
FGGGTKVEIKRTV

SEQ ID NO: 367 - protein, 120 aa, Artificial Sequence
  Description of Artificial Sequence Synthetic polypeptide
EVQLVESGGGLVQPGGSLRLSCAASGFTFSSYRMSWVRQAPGKGLEWVSGIGRSGGRTRYADSVKGRFTISRDNSKNTLYLQMNSLRAEDTAVYYCARAIAAGRYGMDVWGKGTTVTVSS

SEQ ID NO: 368 - protein, 111 aa, Artificial Sequence
  Description of Artificial Sequence Synthetic polypeptide
DIMLTQSPGTLSLSPGERATLSCRASQSIRNNYLAWYQQRPGQAPRLLIYGASYRATGIPDRFSGSGSGTDFTLTISSLEPEDFAVYYCQQRSNWPPTFGGGTKVEIKRTV

SEQ ID NO: 369 - protein, 5 aa, Artificial Sequence
  Description of Artificial Sequence Synthetic peptide
RYPMV

SEQ ID NO: 370 - protein, 17 aa, Artificial Sequence
  Description of Artificial Sequence Synthetic peptide
RISRSGGRTQYADSVKG

SEQ ID NO: 371 - protein, 12 aa, Artificial Sequence
  Description of Artificial Sequence Synthetic peptide
DATGRHYNGMDV

SEQ ID NO: 372 - protein, 11 aa, Artificial Sequence
  Description of Artificial Sequence Synthetic peptide
RASQSISSYLN

SEQ ID NO: 373 - protein, 7 aa, Artificial Sequence
  Description of Artificial Sequence Synthetic peptide
GASSLQS

SEQ ID NO: 374 - protein, 9 aa, Artificial Sequence
  Description of Artificial Sequence Synthetic peptide
QQANSFPLT

SEQ ID NO: 375 - protein, 30 aa, Artificial Sequence; identical to SEQ ID NO: 87
  Description of Artificial Sequence Synthetic polypeptide

SEQ ID NO: 376 - protein, 14 aa, Artificial Sequence
  Description of Artificial Sequence Synthetic peptide
WVRQAPGKGLEWVS

SEQ ID NO: 377 - protein, 32 aa, Artificial Sequence; identical to SEQ ID NO: 9
  Description of Artificial Sequence Synthetic polypeptide

SEQ ID NO: 378 - protein, 11 aa, Artificial Sequence
  Description of Artificial Sequence Synthetic peptide
WGQGTLVTVSS

SEQ ID NO: 379 - protein, 23 aa, Artificial Sequence
  Description of Artificial Sequence Synthetic peptide
EIAMTQSPSSLSASVGDRVTITC

SEQ ID NO: 380 - protein, 15 aa, Artificial Sequence
  Description of Artificial Sequence Synthetic peptide
WYQQKPGKAPKLLIY

SEQ ID NO: 381 - protein, 32 aa, Artificial Sequence; identical to SEQ ID NO: 61
  Description of Artificial Sequence Synthetic polypeptide

SEQ ID NO: 382 - protein, 13 aa, Artificial Sequence
  Description of Artificial Sequence Synthetic peptide
FGGGTKVEIKRTV

SEQ ID NO: 383 - protein, 121 aa, Artificial Sequence
  Description of Artificial Sequence Synthetic polypeptide
EVQLVESGGGLVQPGGSLRLSCAASGFTFSRYPMVWVRQAPGKGLEWVSRISRSGGRTQYADSVKGRFTISRDNSKNTLYLQMNSLRAEDTAVYYCARDATGRHYNGMDVWGQGTLVTVSS

SEQ ID NO: 384 - protein, 110 aa, Artificial Sequence
  Description of Artificial Sequence Synthetic polypeptide
EIAMTQSPSSLSASVGDRVTITCRASQSISSYLNWYQQKPGKAPKLLIYGASSLQSGVPSRFSGSGSGTDFTLTISSLQPEDFATYYCQQANSFPLTFGGGTKVEIKRTV

SEQ ID NO: 385 - protein, 5 aa, Artificial Sequence
  Description of Artificial Sequence Synthetic peptide
SYRMS

SEQ ID NO: 386 - protein, 17 aa, Artificial Sequence
  Description of Artificial Sequence Synthetic peptide
GIGRSGGRTRYADSVKG

SEQ ID NO: 387 - protein, 11 aa, Artificial Sequence
  Description of Artificial Sequence Synthetic peptide
AIAAGRYGMDV

SEQ ID NO: 388 - protein, 12 aa, Artificial Sequence
  Description of Artificial Sequence Synthetic peptide
RASQSIRNNYLA

SEQ ID NO: 389 - protein, 7 aa, Artificial Sequence
  Description of Artificial Sequence Synthetic peptide
GASYRAT

SEQ ID NO: 390 - protein, 9 aa, Artificial Sequence
  Description of Artificial Sequence Synthetic peptide
QQRSNWPPT

SEQ ID NO: 391 - protein, 30 aa, Artificial Sequence; identical to SEQ ID NO: 87
  Description of Artificial Sequence Synthetic polypeptide

SEQ ID NO: 392 - protein, 14 aa, Artificial Sequence
  Description of Artificial Sequence Synthetic peptide
WVRQAPGKGLEWVS

SEQ ID NO: 393 - protein, 32 aa, Artificial Sequence; identical to SEQ ID NO: 9
  Description of Artificial Sequence Synthetic polypeptide

SEQ ID NO: 394 - protein, 11 aa, Artificial Sequence
  Description of Artificial Sequence Synthetic peptide
WGKGTTVTVSS

SEQ ID NO: 395 - protein, 23 aa, Artificial Sequence
  Description of Artificial Sequence Synthetic peptide
EIELTQSPGTLSLSPGERATLSC

SEQ ID NO: 396 - protein, 15 aa, Artificial Sequence
  Description of Artificial Sequence Synthetic peptide
WYQQRPGQAPRLLIY

SEQ ID NO: 397 - protein, 32 aa, Artificial Sequence; identical to SEQ ID NO: 365
  Description of Artificial Sequence Synthetic polypeptide

SEQ ID NO: 398 - protein, 13 aa, Artificial Sequence
  Description of Artificial Sequence Synthetic peptide
FGGGTKVEIKRTV

SEQ ID NO: 399 - protein, 120 aa, Artificial Sequence; identical to SEQ ID NO: 367
  Description of Artificial Sequence Synthetic polypeptide

SEQ ID NO: 400 - protein, 111 aa, Artificial Sequence
  Description of Artificial Sequence Synthetic polypeptide
EIELTQSPGTLSLSPGERATLSCRASQSIRNNYLAWYQQRPGQAPRLLIYGASYRATGIPDRFSGSGSGTDFTLTISSLEPEDFAVYYCQQRSNWPPTFGGGTKVEIKRTV

SEQ ID NO: 401 - protein, 5 aa, Artificial Sequence
  Description of Artificial Sequence Synthetic peptide
RYRMA

SEQ ID NO: 402 - protein, 17 aa, Artificial Sequence
  Description of Artificial Sequence Synthetic peptide
GISYSGGETLYADSVKG

SEQ ID NO: 403 - protein, 10 aa, Artificial Sequence
  Description of Artificial Sequence Synthetic peptide
DVRWLQGLDN

SEQ ID NO: 404 - protein, 16 aa, Artificial Sequence
  Description of Artificial Sequence Synthetic peptide
RSSQSLLHTNGNNYLD

SEQ ID NO: 405 - protein, 7 aa, Artificial Sequence
  Description of Artificial Sequence Synthetic peptide
LGSNRAS

SEQ ID NO: 406 - protein, 9 aa, Artificial Sequence
  Description of Artificial Sequence Synthetic peptide
MQTLQTPLT

SEQ ID NO: 407 - protein, 30 aa, Artificial Sequence; identical to SEQ ID NO: 87
  Description of Artificial Sequence Synthetic polypeptide

SEQ ID NO: 408 - protein, 14 aa, Artificial Sequence
  Description of Artificial Sequence Synthetic peptide
WVRQAPGKGLEWVS

SEQ ID NO: 409 - protein, 32 aa, Artificial Sequence; identical to SEQ ID NO: 9
  Description of Artificial Sequence Synthetic polypeptide

SEQ ID NO: 410 - protein, 11 aa, Artificial Sequence
  Description of Artificial Sequence Synthetic peptide
WGQGTLVTVSS

SEQ ID NO: 411 - protein, 23 aa, Artificial Sequence
  Description of Artificial Sequence Synthetic peptide
EIKLTQSPLSLPVTPGEPASISC

SEQ ID NO: 412 - protein, 15 aa, Artificial Sequence
  Description of Artificial Sequence Synthetic peptide
WYLQKPGQSPQLLIY

SEQ ID NO: 413 - protein, 32 aa, Artificial Sequence; identical to SEQ ID NO: 189
  Description of Artificial Sequence Synthetic polypeptide

SEQ ID NO: 414 - protein, 13 aa, Artificial Sequence
  Description of Artificial Sequence Synthetic peptide
FGGGTKVEIKRTV

SEQ ID NO: 415 - protein, 119 aa, Artificial Sequence
  Description of Artificial Sequence Synthetic polypeptide
EVQLVESGGGLVQPGGSLRLSCAASGFTFSRYRMAWVRQAPGKGLEWVSGISYSGGETLYADSVKGRFTISRDNSKNTLYLQMNSLRAEDTAVYYCARDVRWLQGLDNWGQGTLVTVSS

SEQ ID NO: 416 - protein, 115 aa, Artificial Sequence
  Description of Artificial Sequence Synthetic polypeptide
EIKLTQSPLSLPVTPGEPASISCRSSQSLLHTNGNNYLDWYLQKPGQSPQLLIYLGSNRASGVPDRFSGSGSGTDFTLKISRVEAEDVGVYYCMQTLQTPLTFGGGTKVEIKRTV

SEQ ID NO: 417 - protein, 5 aa, Artificial Sequence
  Description of Artificial Sequence Synthetic peptide
SYAMS

SEQ ID NO: 418 - protein, 17 aa, Artificial Sequence
  Description of Artificial Sequence Synthetic peptide
AISGSGGSTYYADSVKG

SEQ ID NO: 419 - protein, 9 aa, Artificial Sequence
  Description of Artificial Sequence Synthetic peptide
EGRPGYMDV

SEQ ID NO: 420 - protein, 11 aa, Artificial Sequence
  Description of Artificial Sequence Synthetic peptide
RTSLSIATYLH

SEQ ID NO: 421 - protein, 7 aa, Artificial Sequence
  Description of Artificial Sequence Synthetic peptide
HASSLQT

SEQ ID NO: 422 - protein, 9 aa, Artificial Sequence
  Description of Artificial Sequence Synthetic peptide
QQSYSSPYT

SEQ ID NO: 423 - protein, 30 aa, Artificial Sequence; identical to SEQ ID NO: 87
  Description of Artificial Sequence Synthetic polypeptide

SEQ ID NO: 424 - protein, 14 aa, Artificial Sequence
  Description of Artificial Sequence Synthetic peptide
WVRQAPGKGLEWVS

SEQ ID NO: 425 - protein, 32 aa, Artificial Sequence; identical to SEQ ID NO: 9
  Description of Artificial Sequence Synthetic polypeptide

SEQ ID NO: 426 - protein, 11 aa, Artificial Sequence
  Description of Artificial Sequence Synthetic peptide
WGQGTLVTVSS

SEQ ID NO: 427 - protein, 23 aa, Artificial Sequence
  Description of Artificial Sequence Synthetic peptide
EIVLTQSPSLLSASVGDRVTITC

SEQ ID NO: 428 - protein, 15 aa, Artificial Sequence
  Description of Artificial Sequence Synthetic peptide
WYQQKPGRAPKLLIY

SEQ ID NO: 429 - protein, 32 aa, Artificial Sequence
  Description of Artificial Sequence Synthetic polypeptide
GVPSRFSGSGSGTDFTLTISSLLPEDFATYFC

SEQ ID NO: 430 - protein, 13 aa, Artificial Sequence
  Description of Artificial Sequence Synthetic peptide
FGRGTKLEIKRTV

SEQ ID NO: 431 - protein, 118 aa, Artificial Sequence
  Description of Artificial Sequence Synthetic polypeptide
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAREGRPGYMDVWGQGTLVTVSS

SEQ ID NO: 432 - protein, 110 aa, Artificial Sequence
  Description of Artificial Sequence Synthetic polypeptide
EIVLTQSPSLLSASVGDRVTITCRTSLSIATYLHWYQQKPGRAPKLLIYHASSLQTGVPSRFSGSGSGTDFTLTISSLLPEDFATYFCQQSYSSPYTFGRGTKLEIKRTV

SEQ ID NO: 433 - protein, 5 aa, Artificial Sequence
  Description of Artificial Sequence Synthetic peptide
VYGMI

SEQ ID NO: 434 - protein, 17 aa, Artificial Sequence
  Description of Artificial Sequence Synthetic peptide
GIPPSGGVTLYADSVKG

SEQ ID NO: 435 - protein, 7 aa, Artificial Sequence
  Description of Artificial Sequence Synthetic peptide
GNYGMDV

SEQ ID NO: 436 - protein, 11 aa, Artificial Sequence
  Description of Artificial Sequence Synthetic peptide
RASQSVSSYLA

SEQ ID NO: 437 - protein, 7 aa, Artificial Sequence
  Description of Artificial Sequence Synthetic peptide
DASNRAT

SEQ ID NO: 438 - protein, 9 aa, Artificial Sequence
  Description of Artificial Sequence Synthetic peptide
QQRSNWPPT

SEQ ID NO: 439 - protein, 30 aa, Artificial Sequence; identical to SEQ ID NO: 87
  Description of Artificial Sequence Synthetic polypeptide

SEQ ID NO: 440 - protein, 14 aa, Artificial Sequence
  Description of Artificial Sequence Synthetic peptide
WVRQAPGKGLEWVS

SEQ ID NO: 441 - protein, 32 aa, Artificial Sequence; identical to SEQ ID NO: 9
  Description of Artificial Sequence Synthetic polypeptide

SEQ ID NO: 442 - protein, 11 aa, Artificial Sequence
  Description of Artificial Sequence Synthetic peptide
WGQGTLVTVSS

SEQ ID NO: 443 - protein, 23 aa, Artificial Sequence
  Description of Artificial Sequence Synthetic peptide
EIALTQSPATLSLSPGERATLSC

SEQ ID NO: 444 - protein, 15 aa, Artificial Sequence
  Description of Artificial Sequence Synthetic peptide
WYQQKPGQAPRLLIY

SEQ ID NO: 445 - protein, 32 aa, Artificial Sequence; identical to SEQ ID NO: 205
  Description of Artificial Sequence Synthetic polypeptide

SEQ ID NO: 446 - protein, 13 aa, Artificial Sequence
  Description of Artificial Sequence Synthetic peptide
FGGGTKVEIKRTV

SEQ ID NO: 447 - protein, 116 aa, Artificial Sequence
  Description of Artificial Sequence Synthetic polypeptide
EVQLVESGGGLVQPGGSLRLSCAASGFTFSVYGMIWVRQAPGKGLEWVSGIPPSGGVTLYADSVKGRFTISRDNSKNTLYLQMNSLRAEDTAVYYCARGNYGMDVWGKGTTVTVSS

SEQ ID NO: 448 - protein, 110 aa, Artificial Sequence
  Description of Artificial Sequence Synthetic polypeptide
EIALTQSPATLSLSPGERATLSCRASQSVSSYLAWYQQKPGQAPRLLIYDASNRATGIPARFSGSGSGTDFTLTISSLEPEDFAVYYCQQRSNWPPTFGGGTKVEIKRTV

SEQ ID NO: 449 - protein, 5 aa, Artificial Sequence
  Description of Artificial Sequence Synthetic peptide
NYPMT

SEQ ID NO: 450 - protein, 17 aa, Artificial Sequence
  Description of Artificial Sequence Synthetic peptide
TIRGSGGDTWYADSVKG

SEQ ID NO: 451 - protein, 6 aa, Artificial Sequence
  Description of Artificial Sequence Synthetic peptide
WVGRDA

SEQ ID NO: 452 - protein, 16 aa, Artificial Sequence
  Description of Artificial Sequence Synthetic peptide
RSSQSLVYSDGNTYLN

SEQ ID NO: 453 - protein, 7 aa, Artificial Sequence
  Description of Artificial Sequence Synthetic peptide
KVSNRDS

SEQ ID NO: 454 - protein, 9 aa, Artificial Sequence
  Description of Artificial Sequence Synthetic peptide
MQGTHWPYT

SEQ ID NO: 455 - protein, 30 aa, Artificial Sequence; identical to SEQ ID NO: 87
  Description of Artificial Sequence Synthetic polypeptide

SEQ ID NO: 456 - protein, 14 aa, Artificial Sequence
  Description of Artificial Sequence Synthetic peptide
WVRQAPGKGLEWVS

SEQ ID NO: 457 - protein, 32 aa, Artificial Sequence; identical to SEQ ID NO: 9
  Description of Artificial Sequence Synthetic polypeptide

SEQ ID NO: 458 - protein, 11 aa, Artificial Sequence
  Description of Artificial Sequence Synthetic peptide
WGQGTLVTVSS

SEQ ID NO: 459 - protein, 23 aa, Artificial Sequence
  Description of Artificial Sequence Synthetic peptide
DIQLTQSPLSLPVTLGQPASISC

SEQ ID NO: 460 - protein, 15 aa, Artificial Sequence
  Description of Artificial Sequence Synthetic peptide
WFQQRPGQSPRRLIY

SEQ ID NO: 461 - protein, 32 aa, Artificial Sequence
  Description of Artificial Sequence Synthetic polypeptide
GVPDRFSGSVSGPDFTLKISRVEAEDVGVYYC

SEQ ID NO: 462 - protein, 13 aa, Artificial Sequence
  Description of Artificial Sequence Synthetic peptide
FGQGTKLEIKRTV

SEQ ID NO: 463 - protein, 115 aa, Artificial Sequence; identical to SEQ ID NO: 271
  Description of Artificial Sequence Synthetic polypeptide

SEQ ID NO: 464 - protein, 115 aa, Artificial Sequence
  Description of Artificial Sequence Synthetic polypeptide
DIQLTQSPLSLPVTLGQPASISCRSSQSLVYSDGNTYLNWFQQRPGQSPRRLIYKVSNRDSGVPDRFSGSVSGPDFTLKISRVEAEDVGVYYCMQGTHWPYTFGQGTKLEIKRTV

SEQ ID NO: 465 - protein, 18 aa, Artificial Sequence
  Description of Artificial Sequence Synthetic peptide
MHSSALLCCLVLLTGVRA

SEQ ID NO: 466 - protein, 104 aa, Homo sapiens
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 467 - protein, 330 aa, Homo sapiens [sequence omitted]

SEQ ID NO: 468 - protein, 552 aa, Unknown
  Description of Unknown IGSF8 ECD sequence
REVLVPEGPLYRVAGTAVSISCNVTGYEGPAQQNFEWFLYRPEAPDTALGIVSTKDTQFSYAVFKSRVVAGEVQVQRLQGDAVVLKIARLQAQDAGIYECHTPSTDTRYLGSYSGKVELRVLPDVLQVSAAPPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDT